(19) United States
(12) Patent Application Publication
(10) Pub. No.: US 2025/0092411 A1
(43) Pub. Date: Mar. 20, 2025

(54) METHODS FOR ALTERING STARCH GRANULE PROFILE

(71) Applicant: Plant Bioscience Limited, Norwich (GB)

(72) Inventors: David SEUNG, Norwich (GB); Jiawen CHEN, Norwich (GB)

(21) Appl. No.: 18/958,360

(22) Filed: Nov. 25, 2024

Related U.S. Application Data
(62) Division of application No. 17/279,978, filed on Mar. 25, 2021, now Pat. No. 12,209,248.

(30) Foreign Application Priority Data
Sep. 26, 2018 (GB) 1815672.9

(51) Int. Cl. C12N 15/82; C12N 15/11; C12Q 1/6895

(52) U.S. Cl. CPC C12N 15/8245; C12N 15/11; C12N 15/8216; C12Q 1/6895; C12Q 2600/13; C12Q 2600/156

(57) ABSTRACT

The invention relates to methods for altering the size distribution of starch granules in starch storage organs. Also described are genetically altered plants characterised by the above phenotype as well as methods of producing such plants.

### CROSS-REFERENCE TO RELATED APPLICATIONS

This application is a divisional application of U.S. patent application Ser. No. 17/279,978 filed Mar. 25, 2021 which is the U.S. National phase application corresponding to PCT/GB2019/052727 which was assigned an international filing date of Sep. 26, 2019 and associated with publication WO 2020/065331 A1 and which claims priority to GB 1815672.9, filed on Sep. 26, 2018, the disclosures of which are expressly incorporated herein by reference.

### REFERENCE TO A SEQUENCE LISTING SUBMITTED VIA EFS-WEB

The application contains a Sequence Listing which has been submitted electronically in .XML format and is hereby incorporated by reference in its entirety. Said .XML copy, created on Nov. 22, 2024, is named “5609.145467.xml” and is 219,822 bytes in size. The sequence listing contained in this .XML file is part of the specification and is hereby incorporated by reference herein in its entirety.

### FIELD OF THE INVENTION

The invention relates to methods for altering the size distribution of starch granules in starch storage organs. Also described are genetically altered plants characterised by the above phenotype as well as methods of producing such plants.

### BACKGROUND OF THE INVENTION

Starch is a vital source of calories in human diets and an important industrial raw material for the manufacture of many food and non-food products (such as biofuels, paper, pharmaceuticals and textiles) (Jobling, 2004; Smith, 2008; Santelia and Zeeman, 2010). Most plants store a portion of carbon fixed through photosynthesis during the day as starch in leaf chloroplasts. This starch is then degraded to provide energy for growth and metabolism at night. Some plants also accumulate high levels of starch in amyloplasts (i.e. non-photosynthetic chloroplasts for starch storage) of seeds and storage organs. Starch is the major carbohydrate component of many of our staple crops, including cereal grains (wheat, barley, maize, rice, rye, oat), tubers and storage roots (potato, cassava, yam, sweet potato), and banana fruits.

Native starch exists as insoluble, semi-crystalline granules that are composed of two distinct glucose polymers, amylopectin and amylose (Zeeman et al., 2010; Pfister and Zeeman, 2016; Goren et al., 2018). Amylopectin is the major component of the starch granule and is a highly branched polymer consisting of α-1,4-linked glucan chains with α-1,6-linked branch points. The structure of amylopectin facilitates the formation of double helices between adjacent branches, which form the crystalline regions of the starch granule. Amylose consists of long linear α-1,4-linked chains with very few branches, and is thought to reside in the more amorphous regions of the starch granule. The ratio of amylopectin to amylose, as well as the size and shape of starch granules vary greatly depending on botanical source and organ. In Arabidopsis leaves, starch granules are approximately 1 μm in diameter, and contain about 8-10% amylose (Zeeman et al., 2002; Seung et al., 2015). Cereal starches typically contain approximately 15-30% amylose, but the morphology of the granules vary greatly between species (Jane, 1994; Jane et al., 1999). In rice and maize, starch granules have a size distribution between 3-8 μm and 5-20 μm respectively (Jane, 1994; Lindeboom et al., 2004). Cereal crops of the Triticeae (wheat, rye and barley) have a bimodal distribution of granule size, with larger A-type granules and smaller B-type granules. In wheat, A-type granules are 20-30 μm in diameter, while B-type granules (which initiate about 10 days after the initiation of A-type granules) are 2-7 μm in diameter (Bechtel et al., 1990; Howard et al., 2011). A-type granules account for more than 70% of wheat endosperm starch by weight, but less than 10% of the granules by number (Lindeboom et al., 2004).

Previously, little was known about the mechanisms that initiate starch granules in plastids, or factors determining the number of granules per plastid, granule shape and size. In Arabidopsis leaves, each chloroplast contains 5-7 starch granules, and there is relatively little variation in this number (Crumpton-Taylor et al., 2012). The STARCH SYNTHASE 4 (SS4) enzyme was the only protein known to be required for proper granule initiation. Most chloroplasts in Arabidopsis mutants lacking SS4 produce one large starch granule rather than as multiple granules (Roldán et al., 2007; Crumpton-Taylor et al., 2013). Recently, we discovered the PROTEIN TARGETING TO STARCH (PTST) family of proteins in Arabidopsis (Seung et al., 2015, 2017), and two members of this family (PTST2 and PTST3) are required for normal starch granule initiation in Arabidopsis chloroplasts (Seung et al., 2017). Most chloroplasts in Arabidopsis mutants deficient in PTST2 have one large granule per chloroplast, while mutants deficient in PTST3 have only a slight reduction in granule number per chloroplast relative to the wild type. However, granule number is more severely reduced in the ptst2 ptst3 double mutant than either single mutant, suggesting some redundancy between PTST2 and PTST3.

There is significant industrial interest in manipulating the distribution of granule sizes within a plant starch storage organ (Lindeboom et al., 2004). Granule size influences the physico-chemical behaviour of starch, particularly during the gelatinisation process (the swelling and disintegration of the starch granule when heated in the presence of water—a process that occurs during cooking and industrial processing of starch).

In particular, on the one hand, larger starch granules tend to have high swelling power and viscosity, while smaller granules tend to provide smoother paste textures (Santelia and Zeeman, 2010). This means that an increased number of small granules (or a narrower granule size distribution) is desirable for the papermaking and plastic industries, and also for use as a binder or carrier material in the pharmaceutical and cosmetics industries (Lindeboom et al., 2004; Santelia and Zeeman, 2010). Small granule starch is also more effectively digested than larger granules, due to their larger surface area to volume ratio (Dhital et al., 2010). Thus, increasing the number of small starch granules may be desirable for applications where complete and efficient starch digestion is required (e.g. animal feed or bioethanol production). Small granule starches have distinct textural properties that can impart a smooth, cream-like mouth feel to foods, and is particularly suited to carbohydrate-based fat replacers (Lindeboom et al., 2004). While smaller starch granules in general have lower swelling power than larger granules, wheat B-type granules have a higher rate of water absorption than A-type granules (Chiotelli and LeMeste, 2002), possibly due to differences in polymer arrangement or granule morphology. Having more of the small B-type granules in wheat starch positively affects pasta quality, due to the higher rate of water absorption (Soh et al., 2006).

On the other hand, decreasing the number of small/smaller granules (or increasing the size and/or number of the large/larger granules) can be beneficial in certain applications and may improve grain quality. Aside from the higher swelling power of large granules mentioned above, small granules are easily lost during some wet-processing procedures, which results in starch loss and difficult waste management (Stoddard & Starker, 2000). Increasing the overall granule size may reduce the available surface area for digestion, which could reduce the glycemic index of food. In species that have bimodal starch granules, reducing the content of small B-granules would result in a more unimodal size distribution of granules. The bimodal distribution of starch granule size in wheat and barley also causes processing problems, particularly in the brewing industry (Langeveld et al., 2000; Howard et al., 2011). Fewer small granules has also been proposed to be beneficial for beer making (Howard et al., 2011).

Thus, the distribution of granule sizes in a plant starch storage organ is an important parameter for end-use quality. However, the specific changes required to improve this quality will depend on the particular application. Approaches to manipulate the distribution of granule sizes in plants have been limited by our lack of basic knowledge of the starch biosynthesis process. In particular, we did not understand how starch granules initiate, and how the number of granules per plastid is controlled. Granule number per plastid is directly linked to granule size—the same amount of starch can be made as fewer larger granules, or more smaller granules.

There is therefore a need to understand the mechanism controlling the initiation of starch granules in plants, and furthermore to be able to manipulate granule size distribution as required. The present invention addresses this need.

### SUMMARY OF THE INVENTION

We have identified that modulating the expression or activity of at least one MRC (MYOSIN-RESEMBLING CHLOROPLAST PROTEIN) nucleic acid or polypeptide affects starch granule initiation and leads to a change in granule starch distribution. Specifically, we have found that mutating the MRC gene in the endosperm can be used to shift the distribution of granule sizes present in a starch storage organ towards the smaller or the larger granule size, resulting in either smaller granules or larger granules.

The effect of modulating the activity of MRC on the distribution of granule sizes in the endosperm was unexpected from the published research in Arabidopsis leaves (Seung et al. 2018; Vandromme et al., 2018). Multiple granules form in chloroplasts of wild-type Arabidopsis, whereas a single starch granule forms in most chloroplasts of the Arabidopsis mrc mutant. The reduced number of starch granules observed in the loss-of-function mrc mutant suggests that MRC promotes granule initiation in Arabidopsis chloroplasts. By contrast, we have identified wheat mrc mutants that have the same amount of total starch in grains, but produce vast numbers of B-type granules, and an overall increase in the number of granules per grain. Here we report on an unexpected role of MRC in the temporal control of granule initiation in the endosperm. Wheat mrc mutants initiated B-type granules very early during endosperm development (already observed at 10 dpa), leading to a highly altered starch granule size distribution relative to the wild type. Our results reveal a key difference in the granule initiation mechanism between leaves and endosperm, and reveal MRC as a major biotechnological target for the genetic modification of the distribution of starch granule size.

In one aspect of the invention there is provided a method for altering starch granule size distribution in a starch storage organ, the method comprising altering the expression of at least one MRC (MYOSIN-RESEMBLING CHLOROPLAST PROTEIN) nucleic acid and/or altering the activity of a MRC polypeptide, wherein the plant is not Arabidopsis.

In one embodiment, the method comprises altering the distribution of granule size in a plant storage organ, such as a grain or tuber. In a further embodiment, the method comprises altering the granule size distribution in a plastid of a plant storage organ.

In one embodiment, altering the granule size distribution comprises shifting the granule size distribution towards smaller granules or larger granules. In an additional or alternative embodiment, altering the granule size distribution comprises increasing or decreasing the mean granule size in a population of starch granules.

Preferably, the method comprises altering the expression of at least one MRC nucleic acid and/or altering the activity of a MRC polypeptide in at least one plastid, preferably an amyloplast. In a preferred embodiment, the method comprises reducing or abolishing the expression of at least one MRC nucleic acid and/or reducing or abolishing the activity of a MRC polypeptide in at least one plastid. In an alternative embodiment, the method comprises increasing the expression of at least one MRC nucleic acid and/or increasing the activity of a MRC polypeptide in at least one plastid, preferably an amyloplast.

In another aspect of the invention, there is provided a method of altering a physiochemical property of starch, the method comprising altering the expression of at least one MRC nucleic acid and/or altering the activity of a MRC polypeptide. Preferably, the physiochemical property is selected from gelatinisation temperature, swelling power and viscosity.

In one embodiment, the method comprises introducing at least one mutation into at least one nucleic acid sequence encoding a MRC polypeptide. In one embodiment the mutation is a gain of function mutation. In an alternative embodiment, the mutation is a loss of function mutation.

In one embodiment, the nucleic acid sequence encodes a MRC polypeptide as defined in one of SEQ ID NO: 1 to 3 or 29 to 30 or a functional variant or homolog thereof. More preferably, the nucleic acid sequence comprises a sequence selected from SEQ ID NO: 4 to 9 or 31 to 34 or a functional variant or homolog thereof.

In one embodiment, the mutation is introduced using targeted genome modification, preferably ZFNs, TALENs or CRISPR/Cas9 (or Cpf1). Alternatively, the mutation is introduced using mutagenesis, preferably TILLING or T-DNA insertion.

In a further alternative embodiment, the method comprises using RNA interference to reduce or abolish the expression of at least one MRC nucleic acid.

Preferably, the level of MRC expression and/or activity is reduced compared to a control or wild-type plant. More preferably, the starch granule size distribution in a plant is altered compared to a control or wild-type plant.

In one embodiment, the plant may be selected from wheat, barley, rye, maize, potato, sorghum and rice. Most preferably the plant is wheat.

In another aspect of the invention there is provided a genetically altered plant, part thereof or plant cell, wherein said plant is characterised by altered expression of at least one MRC nucleic acid and/or altered activity of a MRC polypeptide, wherein the plant is not Arabidopsis. In one embodiment, the genetically altered plant, part thereof or plant cell is characterised by reduced or abolished expression of at least one MRC nucleic acid and/or reduced or abolished activity of a MRC polypeptide, wherein the plant is not Arabidopsis and wherein the reduced or abolished expression or activity is compared to a control or wild-type plant. In an alternative embodiment, the genetically altered plant, part thereof or plant cell is characterised by increased expression of at least one MRC nucleic acid and/or increased activity of a MRC polypeptide, wherein the plant is not Arabidopsis and wherein the increased expression or activity is compared to a control or wild-type plant.

In one embodiment, the plant comprises at least one mutation in at least one nucleic acid encoding a MRC polypeptide. In one embodiment, said mutation is a complete or partial loss of function mutation. In an alternative embodiment, said mutation is a gain of function mutation.

Preferably the plant is also characterised by an alteration in starch granule size distribution. In one embodiment, the plant is characterised by a shift in the granule size distribution towards smaller or larger granules. In an alternative embodiment, the plant is characterised by an increase or decrease in the mean granule size in a population of starch granules.

In a further embodiment, the mutation is introduced using targeted genome modification, preferably ZFNs, TALENs or CRISPR/Cas9. Alternatively, the mutation is introduced using mutagenesis, preferably TILLING or T-DNA insertion.

In one embodiment, the nucleic acid sequence encodes a MRC polypeptide as defined in one of SEQ ID NO: 1 to 3 or 29 or 30 or a functional variant or homolog thereof. More preferably, the nucleic acid sequence comprises a sequence selected from SEQ ID NO: 4 to 9 or 31 to 34 or a functional variant or homolog thereof.

In a further alternative embodiment, the plant comprises an RNA interference construct that reduces or abolishes the expression of at least one MRC nucleic acid.

In one embodiment, the plant may be selected from wheat, barley, rye, maize, potato, sorghum and rice. Most preferably the plant is wheat. In a preferred embodiment, the plant part is grain or a seed.

In another aspect of the invention, there is provided a method of producing a plant with an alteration in starch granule size distribution, the method comprising altering the expression of at least one MRC nucleic acid and/or altering the activity of a MRC polypeptide, wherein the plant is not Arabidopsis.

In one embodiment, the method comprises reducing or abolishing the expression of at least one MRC nucleic acid and/or reducing or abolishing the activity of a MRC polypeptide in at least one plastid, preferably an amyloplast. In an alternative embodiment, the method comprises increasing the expression of at least one MRC nucleic acid and/or increasing the activity of a MRC polypeptide in at least one plastid, preferably an amyloplast.

In another embodiment, the method comprises introducing at least one mutation into at least one nucleic acid sequence encoding a MRC polypeptide. Preferably the mutation is introduced using targeted genome modification, preferably ZFNs, TALENs or CRISPR/Cas9. Alternatively, the mutation is introduced using mutagenesis, preferably TILLING or T-DNA insertion.

In one embodiment, the nucleic acid sequence encodes a MRC polypeptide as defined in one of SEQ ID NO: 1 to 3 or 29 or 30 or a functional variant or homolog thereof. More preferably, the nucleic acid sequence comprises a sequence selected from SEQ ID NO: 4 to 9 or 31 to 34 or a functional variant or homolog thereof.

In another embodiment, the method comprises using RNA interference to reduce or abolish the expression of at least one MRC nucleic acid.

Preferably, the level of MRC expression and/or activity is reduced or abolished compared to a control or wild-type plant. More preferably, the starch granule size distribution in a plant is altered compared to a control or wild-type plant.

In a further embodiment, the method further comprises measuring an alteration in starch granule size distribution. More preferably, the method further comprises regenerating a plant and screening for an alteration in starch granule size distribution.

In another aspect of the invention there is provided a plant, plant part or plant cell obtained or obtainable by any of the methods described herein.

There is also provided grain derived from the genetically altered plant as described herein. Preferably, the grain is characterised by an altered starch granule size distribution compared to a control or wild-type plant. More preferably, the grain comprises at least one mutation in at least one nucleic acid encoding a MRC gene and/or at least one mutation in a MRC promoter.

In another aspect of the invention, there is provided starch obtained or obtainable from at least one plant cell of the genetically altered plant as described herein or the grain described herein. There is also provided a food or feed composition prepared from the grain as described herein or the starch described herein.

In another aspect of the invention, there is provided the use of the grain as described herein or the starch as described herein as a food or feedstuff. Alternatively, there is provided the use of the grain as described herein or the starch as described herein in any pharmaceutical or industrial application.

In another aspect of the invention there is provided a nucleic acid construct comprising a nucleic acid sequence encoding at least one DNA-binding domain or protospacer element that can bind to at least one target sequence in a MRC gene and/or promoter, wherein preferably the target sequence is selected from SEQ ID NO: 39 to 42 or a variant thereof.

In a preferred embodiment, the sequence of the protospacer element is selected from SEQ ID Nos 43 to 46 or a variant thereof.

In a further preferred embodiment, the construct further comprises a nucleic acid sequence encoding a CRISPR RNA (crRNA) sequence, wherein said crRNA sequence comprises at least one protospacer element sequence and additional nucleotides. More preferably, the construct further comprises a nucleic acid sequence encoding a transactivating RNA (tracrRNA), wherein preferably the tracrRNA is defined in SEQ ID NO.47 or a functional variant thereof.

In another aspect of the invention there is provided a nucleic acid construct, wherein the construct encodes at least one single-guide RNA (sgRNA), wherein said sgRNA comprises the tracrRNA sequence and the crRNA or protospacer sequence, wherein the sgRNA comprises or consists of a sequence selected from SEQ ID Nos 48 to 51 or a functional variant thereof.

In any of the above described constructs, the construct comprises a promoter, wherein preferably the promoter is operably linked to the protospacer element or sgRNA nucleic acid sequence. Preferably, the promoter is a constitutive promoter.

In a further embodiment, the nucleic acid construct further comprises a nucleic acid sequence encoding a CRISPR enzyme. Preferably, the CRISPR enzyme is a Cas or Cpf1 protein. More preferably, the Cas protein is Cas9 or a functional variant thereof.

In an alternative embodiment, the nucleic acid construct encodes a TAL effector. Preferably, the nucleic acid construct further comprises a sequence encoding an endonuclease or DNA-cleavage domain thereof. More preferably, the endonuclease is FokI.

In another aspect of the invention, there is provided a single guide (sg) RNA molecule wherein said sgRNA comprises a crRNA sequence and a tracrRNA sequence, wherein the crRNA sequence can bind to at least one sequence selected from SEQ ID Nos 39 to 42 or a variant thereof. Preferably the sgRNA has a RNA sequence comprising or consisting of a sequence selected from SEQ ID NO: 52 to 55 or a functional variant thereof.

In a further aspect there is provided an isolated plant cell transfected with at least one nucleic acid construct as described herein or the sgRNA molecule as described herein.

In one embodiment, the isolated plant cell is transfected with at least one nucleic acid construct comprising a sgRNA nucleic acid sequence and a second nucleic acid construct, wherein said second nucleic acid construct comprises a nucleic acid sequence encoding a Cas protein, preferably a Cas9 or Cpf1 protein or a functional variant thereof. In a preferred embodiment, the second nucleic acid construct is transfected before, after or concurrently with the nucleic acid construct comprising a sgRNA nucleic acid sequence.

In another aspect of the invention, there is provided a genetically modified plant, wherein said plant comprises the transfected cell as defined herein. Preferably, the nucleic acid encoding the sgRNA and/or the nucleic acid encoding a Cas or Cpf1 protein is integrated in a stable form.

In another aspect of the invention, there is provided a method of altering starch granule size distribution in a plant, the method comprising introducing and expressing in a plant the nucleic acid construct as described herein or the sgRNA molecule as described herein, wherein preferably said increase is relative to a control or wild-type plant.

In a further aspect, there is provided a plant obtained or obtainable by any method described herein.

In another aspect, there is provided the use of a nucleic acid construct as described herein or the sgRNA molecule as described herein to alter starch granule size distribution in a plant. Preferably, the nucleic acid construct or sgRNA molecule reduces the expression and/or activity of MRC in a plant.

In a further aspect of the invention, there is provided a method for obtaining the genetically modified plant as described herein, the method comprising:
a. selecting a part of the plant;
b. transfecting at least one cell of the part of the plant of paragraph (a) with the nucleic acid construct as described herein or the sgRNA molecule as described herein; and
c. regenerating at least one plant derived from the transfected cell or cells; selecting one or more plants obtained according to paragraph (c) that show reduced expression and/or activity of at least one MRC nucleic acid in said plant.

In a final aspect of the invention, there is provided a method for identifying and/or selecting a plant that has, or will have, altered starch granule size distribution, preferably compared to a wild-type or control plant, the method comprising detecting in the plant or plant germplasm at least one polymorphism or mutation in the MRC gene and/or MRC promoter and selecting said plant or progeny thereof. Preferably, the polymorphism is an insertion, deletion and/or substitution. More preferably, the method further comprises introgressing the chromosomal region comprising at least one polymorphism in the MRC gene and/or MRC promoter into a second plant or plant germplasm to produce an introgressed plant or plant germplasm

### DESCRIPTION OF THE FIGURES

The invention is further described in the following non-limiting figures:

FIG. 1 shows schematic illustrations of MRC homeologs in wheat. A) Location of TaMRC homeologs on chromosome 6A and 6D. The pink boxes represent TaMRC homeologs, while homeologs of the adjacent genes are shown in green (cytochrome P450 family protein), purple (respiratory burst oxidase homolog) and blue (uncharacterised protein). Arrowheads on the boxes indicate direction of transcription. The syntenic region on chromosome 6B has a large insertion, depicted with a black arrowhead. The diagram is drawn to scale, and chromosome coordinates of the region are indicated. B) Gene models of the TaMRC 6A and 6D homeologs and 6B pseudogene. Exons are represented with pink boxes, while light pink boxes represent the 5′ and 3′ UTRs. The locations of the mutations in the mrc mutants are depicted with red arrows, and the mutated codons/amino acids are shown in red letters.

FIG. 2 shows altered starch granule size distribution of endosperm starch in wheat mrc mutants. A) Thin sections of mature endosperm tissue were stained with Lugol's solution and imaged using light microscopy. Bar=40 μm. B) Purified endosperm starch granules were observed using scanning electron microscopy (SEM). Bar=20 μm. C) Starch granule size distribution of endosperm starch. Size distributions were determined by measuring at least 100,000 granules per replicate with a coulter counter. The solid line shows the mean curve (shading represents the SEM) from three replicate determinations. Each replicate used starch purified from three individual grains, and the three replicates represent grains from three different plants.

FIG. 3 shows the starch content and granule count in developing endosperm tissue. The endosperm was dissected from developing grains of the wild type (WT) and mrc-1 mutant, harvested at 8, 14, 20 and 30 dpa. A) Starch content of the endosperm. Values are expressed relative to the fresh weight of the dissected endosperm, and are mean±SE of measurements from 3-4 endosperms from developing grains harvested from separate plants. B) Starch granule number in the endosperm. Starch was purified from dissected endosperm and the number of granules was determined using a coulter counter. Values are expressed relative to the fresh weight of the dissected endosperm, and are mean±SE of measurements from 3-4 endosperms from developing grains harvested from separate plants.

FIG. 4 shows the starch granule size distributions in developing endosperm tissue. The endosperm was dissected from developing grains of the wild type (WT) and the mrc-1 mutant harvested at 8, 14, 20 and 30 dpa. A) Purified endosperm starch granules were observed using scanning electron microscopy (SEM). Bars=20 μm. B) Starch granule size distribution of endosperm starch characterised with a coulter counter. Distributions are the average of measurements carried out on grains (3 per measurement) harvested from three different plants. The solid line shows the mean curve, while the shading represents the standard error of the mean.

FIG. 5 shows the observation of starch granules within developing endosperm tissue. A) Scanning electron micrographs of developing endosperm tissue subjected to critical point drying. Grains were harvested from the wild type (WT) and mrc-1 mutant at 10, 15 and 20 days post anthesis (dpa). Bars=50 μm. B) Light micrographs of endosperm sections. Semi-thin sections of embedded developing grains (15 dpa) were stained with toluidine blue as a negative stain for starch granules. Examples of A-type granules and B-type granules are marked with arrows respectively. Bars=50 μm. C) Endosperm sections observed using TEM.

FIG. 6 shows a phylogenetic analysis of wheat MRC sequences. A) Phylogenetic tree of MRC orthologs. The amino acid sequences of the orthologs identified in Seung et al. 2018 were aligned with the wheat sequences. A maximum likelihood tree was assembled with 1000 bootstraps. Bootstrap values are shown next to each node. Branch lengths indicate the number of substitutions per site. B) Multiple sequence alignment of MRC orthologs generated with Clustal O. The region surrounding Leu289 (highlighted in yellow) is shown. Symbols under the alignment indicate conserved residues with: complete identity (*), highly similar properties (:) or weakly similar properties (.)C) Coiled coil prediction of TaMRC 6A wild type (WT) sequence and the Leu289Phe (L289F) mutant using COILS/PCOILS. The probability was calculated using the 28 amino acid window.

FIG. 7 shows the 6B pseudogene does not contribute to granule size distribution. Size distributions were determined by measuring at least 100,000 granules per replicate with a Coulter counter. The solid line shows the mean curve (shading represents the SEM) from three replicate determinations. Each replicate used starch purified from three individual grains, and the three replicates represent grains from three different plants. A) Starch from genotypes isolated from the mrc-1 cross: the single A homeolog mutant (aa BB), the single B pseudogene mutant (AA bb), and the double mutant (aa bb). B) Same as A), but with genotypes from the mrc-2 cross.

FIG. 8 shows the chain length distribution and amylose content of the mrc mutants. A) Chain length distribution of mrc-1 and mrc-2 starch. Purified starch was debranched and analysed with High Performance Anion Exchange Chromatography with Pulsed Amperometric Detection (HPAEC-PAD). Relative peak areas show the mean±SEM from three replicate measurements. B) Amylose content of mrc-1 and mrc-2 starch quantified using iodine colourimetry. Values represent mean±SEM from three replicate measurements.

FIG. 9 shows a table of granule size and B-type granule content in mrc mutants. Granule size distributions were calculated using a Coulter counter (n=3 per genotype). The mean size of A-type and B-type granules and the content of B-type granules (by percentage volume) were calculated by fitting a bimodal mixed gaussian curve to the size distributions. Values marked with an asterisk (*) are significantly different to the wild-type value under a two-tailed t-test (p<0.05).

FIG. 10 shows a table of starch content of mature grains from mrc mutants. Note that there are no significant differences in starch content between mutants and the wild type.

FIG. 11 shows a table of wheat mrc mutants from a TILLING database.

FIG. 12 shows a multiple sequence alignment of AtMRC, HvMRC and TaMRC homeologs from Cadenza and Kronos. Symbols under the alignment indicate conserved residues with: complete identity (*), highly similar properties (:) or weakly similar properties (.).

FIG. 13 shows a table of the gelatinisation temperature of mrc-1 aabb starch. Values marked with an asterisk (*) are significantly different from the WT value under a two-tailed t-test at p<0.05.

FIG. 14 shows the size distribution of endosperm starch from mrc mutants in the hexaploid bread wheat cultivar, Cadenza. Starch was purified from mature grains and the size distribution was quantified on a Coulter counter.

FIG. 15 shows swelling power of starches from the mrc mutants. Purified starches were heated in water at 100° C. for 30 mins and allowed to settle for 1 hour at room temperature. (A) Swelling power was calculated as mass after swelling relative to the initial mass. (B) The increase in the average volume of granules (relative to initial volume) throughout a 30 mins incubation in water at 60° C. Volumes were quantified using a Coulter counter. (C) shows starch viscosity of mrc wheat starch compared to wild-type during gelatinisation, measured with a Rapid Visco Analyser (RVA). Two runs were carried out per sample and the shading indicates the area between the two replicate curves.

FIG. 16 shows the glucose content of the endosperm during the initial phase of grain germination. Glucose was measured using the hexokinase/glucose-6-phosphate dehydrogenase method. Values are the mean±SE of measurements from n=6-9 grains. The mrc-1 mutant has significantly higher glucose in the endosperm at day four of germination than the WT and mrc-2 grains under a two-tailed t-test with p<0.05.

FIG. 17 shows the yield and composition of mrc grains. The absence of large significant differences in these parameters suggest that the impact of mrc mutations is specific to starch granule size, and not other important aspects of grain yield and quality. A. Grain yield per plant (in grams) was calculated for WT, mrc-1 and mrc-2. Values are mean±SE. There are no significant differences B-D) Near infrared (NIR) grain analysis was used to determine the percentage of the total grain mass that is moisture (B), protein (C) and starch (D). Bars marked with an asterisk (*) are significantly different to the wild-type under a two-tailed t-test (p<0.05).

FIG. 18 shows the size distribution of endosperm starch from the Kronos2485 mutant compared to the mrc-1 and mrc-3 mutants in Kronos. A) Size distributions were determined using the Coulter counter. The solid line shows the mean curve (shading represents the SEM) from three replicate determinations. Each replicate used starch purified from three individual grains, and the three replicates represent grains from three different plants. B) Table showing the mean sizes of A- and B-type granules, and B-type granule content. Data are derived from the distribution plots in A). Values marked with an asterisk (*) are significantly different to the wild-type under a two-tailed t-test (p<0.05).

FIG. 19 shows the size distribution of endosperm starch from the Kronos2485, Kronos2096 (A) and Kronos775 mutants (B). Starch was purified from mature grains and the size distribution was quantified on a Coulter counter. All three of these mutants have fewer B-type granules than the wild type.

FIG. 20 shows the tissue-specific expression of OsMRC in rice. Publicly available tissue-specific expression data were retrieved for OsMRC (LOC Os02g09340.1) using the rice eFP browser (bar.utoronto.ca). Strong expression of the gene is observed in leaves, as well as in the developing seed—at several different developmental stages: S1: 0-2 dpa, S2: 3-4 dpa, S3: 5-10 dap, S4: 11-20 dpa, and S5: 21-29 dpa.

FIG. 21 is a table showing the gelatinisation temperature of mrc-1 aabb starch. Values marked with an asterisk (*) are significantly different from the WT value under a two-tailed t-test at p<0.05.

FIG. 22 shows the prediction of coiled coils in AtMRC (A) and TaMRC (B) using the COILS program. The position of each amino acid in the protein sequence is plotted on the x-axis, while the probability of forming a coiled coil is plotted on the y-axis. The green line represents probabilities calculated using a 14-amino-acid prediction window, the blue line using a 21-amino-acid prediction window, and the purple line using a 28-amino-acid prediction window.

FIG. 23 is a table showing the expression levels of TaMRC in starchy endosperm during grain development. Publicly available expression data were retrieved from the wheat expression browser (http://www.wheat-expression.com). Values are in transcript per kilobase million (tpm). Values corresponding to low expression (tpm<2) are shown in grey.

FIG. 24 shows that granule size distribution in mrc-1 mutants by microscopy. The AA BB, aa BB, AA bb and aa bb mutants from the mrc-1 set were analysed.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

The aspects of the invention involve recombinant DNA technology and exclude embodiments that are solely based on generating plants by traditional breeding methods.

In a first aspect of the invention, there is provided a method for altering a starch characteristic. Preferably said starch characteristic is starch granule size distribution in a plant, the method comprising altering the expression of at least one MYOSIN-RESMEBLING CHLOROPLAST PROTEIN (referred to herein as “MRC”) nucleic acid and/or altering the activity of a MRC polypeptide. MRC may also be referred to as PROTEIN INVOLVED IN STARCH INITIATION “PII1” and such terms may be used interchangeably. In one embodiment, the plant is not Arabidopsis.

In one embodiment, the method has no (obvious detectable) effect on total starch content and/or plant growth.

The terms “seed” and “grain” as used herein can be used interchangeably. The terms “increase”, “improve” or “enhance” as used herein are also interchangeable. Similarly, the terms starch “grain” or “granule” are also interchangeable.

The present invention has identified methods to modify the properties of starch granules. It is understood that starch granules will have a range of sizes within a starch storage organ. Such a distribution can be considered as a standard or characteristic distribution curve for the plant. The characteristic distribution curve for starch granules will vary plant by plant. The present invention has identified methods to vary the distribution curve of starch granules from the otherwise standard or characteristic distribution. In an embodiment, varying may involve shifting the distribution towards smaller granules. In a different embodiment, varying may involve shifting the distribution towards larger granules. In a yet further embodiment, varying may modify the distribution curve towards a narrower curve, e.g. the standard deviation from the mean granule size is reduced compared against a standard distribution curve. Such a change will result in more uniform granules. This may occur at the same time as shifting the granule size. The present invention therefore alters starch granule size distribution.

In an embodiment, there is provided a method of altering granule size distribution that results in increasing or decreasing the mean granule size in a population of starch granules. In an embodiment, the granule size may be increased or decreased by 3% or more, 5% or more, 10% or more, 15% or more, 20% or more, 25% or more, 30% or more, 35% or more, 40% or more or 45% or more compared to the mean granule size in a control or wild-type plant.

In a yet further embodiment, there is provided a method of altering the standard deviation of starch granule size, particularly reducing the standard deviation thereby leading to more uniform starch granule size. In an embodiment, the standard deviation of starch granule size is reduced by 3% or more, 5% or more, 10% or more, 15% or more, 20% or more, 25% or more, 30% or more, 35% or more, 40% or more or 45% or more compared to the starch granule size standard deviation in a control or wild-type plant.

Granule size may be measured by a number of techniques that would be known to the skilled person. In one embodiment, granule size can be measured using a particle size analyser, which uses laser scattering to measure the total volume of particles of a given size, expressed as a percentage of the total volume of all particles. In one example, purified starch can be suspended in water and measured on a particle size analyser, such as a Beckman-Coulter Multisizer 4e Coulter counter, or the Coulter LS-230 laser-scattering instrument (Beckman Coulter). In another embodiment, granule size can be measured using light microscopy. As described below, starch granule area in the images was measured using the Particle Analysis plugin of ImageJ software (v.2.0.0; https://imagej.net/). The area was used to calculate diameter, assuming the granules were perfect circles. Unlike the first method with the particle size analyser, this method calculates of the percentage of granules with a given size relative to the total number of granules (rather than as volumes as measured on a Coulter counter), and is a direct measure of size (rather than inferred from laser scattering).

As used herein, the terms “reducing” means a decrease in the levels of MRC expression and/or activity by up to or more than 10%, 20%, 30%, 40%, 50%, 60%, 70%, 80% or 90% when compared to the level in a wild-type or control plant. In one embodiment, reducing means a decrease in at least 50% compared to the level in a wild-type or control plant. Reducing may or may not encompass changes in the absolute MRC transcript level, preferably it does not. Reducing also may or may not encompass abolishing expression. The term “abolish” expression means that no expression of MRC is detectable (no transcript) or that no functional MRC polypeptide is produced. Methods for determining the level of MRC expression and/or activity would be well known to the skilled person. These reductions can be measured by any standard technique known to the skilled person. For example, a reduction in the expression and/or content levels of at least MRC expression may be a measure of protein and/or nucleic acid levels and can be measured by any technique known to the skilled person, such as, but not limited to, any form of quantitative PCR, gel electrophoresis and immunoblotting or chromatography (e.g. HPLC). In one embodiment, the mutation is a complete or partial loss-of-function mutation. In one embodiment, the mutation reduces or abolishes the protein-interacting activity of MRC. MRC interacts with SS4 directly, as well as associates with PTST2 and MFP1 (either directly or indirectly). It may also interact with other proteins in the wheat endosperm. Accordingly, the method may comprise measuring these protein-protein interactions, using techniques standard in the art, such as, but not limited to, interaction assays using recombinant proteins, yeast-2-hybrid, immunoprecipitation or bimolecular fluorescence.

As used herein, the terms “increasing” means an increase in the level of MRC expression and/or activity by up to or more than 10%, 20%, 30%, 40%, 50%, 60%, 70%, 80% or 90% when compared to the level in a wild-type or control plant. In one embodiment, increasing means an increase in at least 50% compared to the level in a wild-type or control plant. Increasing may or may not encompass changes in the absolute MRC transcript level, preferably it does not. As described above, methods for determining the level of MRC expression and/or activity would be well known to the skilled person. In one embodiment, increasing the activity of the MRC polypeptide may be achieved by introducing a gain of function mutation. In one example, the gain of function mutation increases the protein-interacting activity of MRC, as described above. Again, an increase in the activity of the MRC polypeptide may be determined by measuring these protein-protein interactions, using techniques standard in the art, such as, but not limited to, interaction assays using recombinant proteins, yeast-2-hybrid, immunoprecipitation or bimolecular fluorescence.

In a particular embodiment, the method comprises reducing or abolishing the expression of at least one MRC nucleic acid and/or reducing or abolishing the activity of a MRC polypeptide in a starch storage organ, such as a tuber or grain. In a further embodiment, the method comprises reducing or abolishing the expression of at least one MRC nucleic acid and/or reducing or abolishing the activity of a MRC polypeptide in an amyloplast preferably in the endosperm of the plant. As a result, the size distribution of starch granules is altered in at least one plastid in a plant. More specifically, reducing or abolishing expression or activity of MRC biases the distribution of granules in favour of the small or smaller sizes (i.e. shifts the distribution curve towards the left). This means that there is in an increase in the number of small granules and a decrease in the number and/or size of the large granules. This can also be considered as a decrease in the mean granule size. As discussed above, the advantages of such a starch granule profile are numerous. For example, such a granule profile may be useful where complete and efficient digestion of starch is required, for example, in animal feed or bioethanol production—or for its distinct textural or swelling properties that can improve quality of the final food product.

In an alternative embodiment, the method comprises increasing the expression of at least one MRC nucleic acid and/or increasing the activity of at least one MRC polypeptide in a plastid, preferably a grain or tuber. In a further embodiment, the method comprises increasing the expression of at least one MRC nucleic acid and/or increasing the activity of a MRC polypeptide in an amyloplast preferably in the endosperm of the plant. As a result, granule size distribution is altered in at least one plastid in a plant, preferably in the endosperm of the plant. More specifically, increasing expression or activity of MRC biases the distribution of granules in favour of the larger granule sizes (i.e. shifts the distribution curve to the right). This means that there is in an increase in the size and/or number of larger granule size. This can also be considered as an increase in the mean granule size. Again, as discussed above, altering the size distribution of granules has numerous benefits, reduced digestibility, increased viscosity of starch gels, and improved milling/processing efficiency.

By “at least one mutation” is meant that where the MRC gene is present as more than one copy or homoeologue (with the same or slightly different sequence) there is at least one mutation in at least one gene. In one embodiment, all genes are mutated. In another embodiment, where the plant is a tetraploid, for example tetraploid wheat, the MRC gene is mutated on the A genome only or the A and B genome. In another embodiment, where the plant is a hexaploid, for example hexaploid wheat, the MRC gene is mutated on the A and/or B and/or D genome or more preferably, the A and D genome only.

In one embodiment, the method comprises introducing at least one mutation into the, preferably endogenous, gene encoding MRC and/or the MRC promoter. Preferably said mutation is in the coding region of the MRC gene. Alternatively, said mutation is in an intronic sequence or the 5′UTR or 3′UTR. In a further embodiment, at least one mutation or structural alteration may be introduced into the MRC promoter such that the MRC gene is either not expressed (i.e. expression is abolished) or expression is reduced, as defined herein. In an alternative embodiment, at least one mutation may be introduced into the MRC gene such that the altered gene does not express a full-length (i.e. expresses a truncated) MRC protein or does not express a fully functional MRC protein. In this manner, the activity of the MRC polypeptide can be considered to be reduced or abolished as described herein. In any case, the mutation may result in the expression of MRC with no, significantly reduced or altered biological activity in vivo. Alternatively, MRC may not be expressed at all.

In an alternative embodiment, at least one mutation may be introduced into the, preferably endogenous, gene encoding MRC and/or the promoter such that the expression of the MRC nucleic acid or the activity of the MRC polypeptide is increased. Such a mutation is called a gain-of function or activating mutation.

In one embodiment, the sequence of the MRC gene comprises or consists of a nucleic acid sequence selected from SEQ ID NO: 4 to 6 (genomic) or 7 to 9 (CDS) or a functional variant or homologue thereof and encodes a polypeptide as defined in one of SEQ ID NO: 1 to 3 respectively or a functional variant or homologue thereof. The genomic DNA sequences of the 6A homeolog (for Kronos and Cadenza) is shown in SEQ ID NO: 4 and 5 respectively, and the 6D (homeolog for Cadenza) is shown in SEQ ID NO: 6. The cDNA sequences are shown in SEQ ID NOs 7 (6A—Kronos), 8 (6A—Cadenza) and 8 (6D-Cadenza). The genomic DNA sequence and the CDS sequence of the 6B homeolog in Kronos is shown in SEQ ID NO: 31 and 33 respectively and in Cadenza in SEQ ID NO: 32 and 34 respectively.

As used throughout, by “MRC promoter” is meant a region extending at least or approx. 1.5 kbp upstream of the ATG codon of the MRC ORF. In one embodiment, the sequence of the MRC promoter comprises or consists of a nucleic acid sequence as defined in any one of SEQ ID NO: 22 to 24 and 35 to 36 or a functional variant or homologue thereof. Examples of promoter homologues are shown in SEQ ID NOs 25 to 28. In one embodiment, the MRC promoter may also include 5′ UTR sequences.

In the above embodiments an ‘endogenous’ nucleic acid may refer to the native or natural sequence in the plant genome. Also included in the scope of this invention are functional variants (as defined herein) and homologs of the above identified sequences. Examples of homologs are shown in SEQ ID NOs: 10 to 21 and 60 to 62. Accordingly, in one embodiment, the homolog encodes a polypeptide selected from SEQ ID NOs: 10, 13, 18 19 and 60; or the homolog comprises or consists of a nucleic acid sequence selected from SEQ ID NOs: 11, 12, 14, 15, 16, 17, 18, 20, 21, 61 and 62.

The term “functional variant” (or “variant”) as used herein with reference to any of the sequences described herein refers to a variant sequence or part of the sequence which retains the biological function of the full non-variant sequence. A functional variant also comprises a variant of the gene of interest which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence which result in the production of a different amino acid at a given site that do not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

In one embodiment, a functional variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

The term homolog, as used herein, also designates a MRC promoter or MRC gene orthologue from other plant species. A homolog may have, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid represented by any of SEQ ID NO: 1 to 3, 29 and 30 or to the nucleic acid sequences as shown by SEQ ID NOs: 4 or 9, 31 to 34. A MRC promoter orthologue may have, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the nucleic acid sequences as shown in SEQ ID NOs 22 to 24, 35 and 36. In one embodiment, overall sequence identity is at least 37%. In one embodiment, overall sequence identity is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 90%, 91%, 92%, 93%, 94%, 95%, 96%, 97%, 98%, or at least 99%.

Functional variants of MRC homologs as defined above are also within the scope of the invention.

MRC does not have any recognisable conserved domains. However, the majority of the MRC protein forms coiled coils, which are alpha-helices that can mediate protein-protein interaction. Coiled coils typically form from a (a-b-c-d-e-f-g)ₙ heptad repeat, where a and d are non-polar resides, and e and g are polar residues (Mason and Arndt, 2004). Coiled coils are therefore variable in sequence as long as the heptad repeat is conserved, and thus, no strictly conserved amino acid motifs were found in the polypeptide sequence of the coiled coils from MRC orthologs. Also, the length and position of coiled coils appears to be variable among AtMRC and TaMRC. The mutation in MRC may reside in the coiled coils, and result in amino acid substitutions that break the coiled coil heptad. Accordingly, in one embodiment the MRC protein comprises at least one coiled coil and at least one mutation is introduced into at least one coiled coil to affect protein function.

Additionally, all higher plant MRC proteins have an L(L/F)(D/E)(K/R)LF motif towards the N-terminal end of the protein, after the chloroplast transit peptide but before the start of the coiled coils. The strict conservation of this motif among MRC orthologs implies that it plays an important role in MRC function. The mutation may encode an amino acid substitution that disrupts the function of this motif. Also, the mutation may reside in the chloroplast transit peptide (the first 17 amino acids of TaMRC). The length of the transit peptide may be predicted for MRC orthologs using ChloroP program (Emanuelsson et al., 2007). The mutation may alter the transit peptide sequence in a way that the MRC polypeptide can no longer be delivered to chloroplasts/amyloplasts.

Accordingly, in a further embodiment, the MRC nucleic acid (coding) sequence encodes a MRC protein with at least one conserved domain with the following sequence:

| | |
|---|---|
| SEQ ID NO: 37: | L X1 X2 X3LF |

where X1 is L or F, X2 is D or E and X3 is K or R
or a variant thereof, wherein the variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to SEQ ID NO: 37.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

Suitable homologues can be identified by sequence comparisons and identifications of conserved domains. There are predictors in the art that can be used to identify such sequences. The function of the homologue can be identified as described herein and a skilled person would thus be able to confirm the function, for example when knocked-out in a plant.

Thus, the nucleotide sequences of the invention and described herein can also be used to isolate corresponding sequences from other organisms, particularly other plants, for example crop plants. In this manner, methods such as PCR, hybridization, and the like can be used to identify such sequences based on their sequence homology to the sequences described herein. Topology of the sequences and the characteristic domains structure can also be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, New York).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than 24 hours, usually about 4 to 12. Stringent conditions may also be achieved with the addition of destabilizing agents such as formamide.

In a further embodiment, a variant as used herein can comprise a nucleic acid sequence encoding a MRC polypeptide as defined herein that is capable of hybridising under stringent conditions as defined herein to a nucleic acid sequence as defined in any of SEQ ID NOs: 4 to 9, 11, 12, 14, 15, 16, 17, 20, 21, 22 to 28, 31 to 37 and 61 to 62.

In one embodiment, the method comprises altering the expression of at least one nucleic acid encoding a MRC polypeptide or altering the activity of an MRC polypeptide, as described herein, wherein the method comprises introducing at least one mutation into at least one MRC gene and/or promoter, wherein the MRC gene comprises or consists of
a. a nucleic acid sequence encoding a polypeptide as defined in one of SEQ ID NOs: 1 to 3, 10, 13, 18, 19, 29 to 30 and 60; or
b. a nucleic acid sequence as defined in one of SEQ ID NOs: 4 to 9, 11, 12, 14, 15, 16, 17, 20, 21, 31 to 34, 61 and 62; or
c. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to either (a) or (b); or
d. a nucleic acid sequence encoding a MRC polypeptide as defined herein that is capable of hybridising under stringent conditions as defined herein to the nucleic acid sequence of any of (a) to (c);
   and wherein the MRC promoter comprises or consists of
e. a nucleic acid sequence as defined in one of SEQ ID NOs: 22 to 28, 35 and 36
f. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to (e); or
g. a nucleic acid sequence capable of hybridising under stringent conditions as defined herein to the nucleic acid sequence of any of (e) to (f).

In one embodiment, a mutation is introduced into two MRC genes, wherein the first MRC gene encodes a protein as defined in SEQ ID NO: 2 (preferably the MRC gene comprises or consists of a nucleic acid sequence as defined in SEQ ID NO: 5 or 8) and wherein the second MRC gene encodes a protein as defined in SEQ ID NO: 3 (preferably the MRC gene comprises or consists of a nucleic acid sequence as defined in SEQ ID NO: 6 or 9). In an alternative embodiment, a mutation is introduced into two MRC genes, wherein the first MRC gene encodes a protein as defined in SEQ ID NO: 1 (preferably the MRC gene comprises or consists of a nucleic acid sequence as defined in SEQ ID NO: 4 or 7) and wherein the second MRC gene encodes a protein as defined in SEQ ID NO: 29 (preferably the MRC gene comprises or consists of a nucleic acid sequence as defined in SEQ ID NO: 31 or 33). In a further embodiment, a mutation is introduced into three MRC genes, wherein the first MRC gene encodes a protein as defined in SEQ ID NO: 2 (preferably the MRC gene comprises or consists of a nucleic acid sequence as defined in SEQ ID NO: 5 or 8) and wherein the second MRC gene encodes a protein as defined in SEQ ID NO: 3 (preferably the MRC gene comprises or consists of a nucleic acid sequence as defined in SEQ ID NO: 6 or 9) and wherein the third MRC gene encodes a protein as defined in SEQ ID NO: 30 (preferably the MRC gene comprises or consists of a nucleic acid sequence as defined in SEQ ID NO: 32 or 34).

In one embodiment, the mutation is a loss-of function mutation. Preferably the loss of function mutation is at the start of the of protein coding sequence (e.g. in the N-terminus of the protein). In one embodiment, the loss of function is in the first 258 amino acids of the MRC polypeptide.

In a preferred embodiment, the mutation that is introduced into the endogenous MRC gene or promoter thereof to alter the biological activity and/or expression levels of the MRC gene or protein can be selected from the following mutation types
1. a “missense mutation”, which is a change in the nucleic acid sequence that results in the substitution of one amino acid for another amino acid;
2. a “nonsense mutation” or “STOP codon mutation”, which is a change in the nucleic acid sequence that results in the introduction of a premature STOP codon and, thus, the termination of translation (resulting in a truncated protein); in plants, the translation stop codons may be selected from “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation.
3. an “insertion mutation” of one or more nucleotides or one or more amino acids, due to one or more codons having been added in the coding sequence of the nucleic acid;
4. a “deletion mutation” of one or more nucleotides or of one or more amino acids, due to one or more codons having been deleted in the coding sequence of the nucleic acid;
5. a “frameshift mutation”, resulting in the nucleic acid sequence being translated in a different frame downstream of the mutation. A frameshift mutation can have various causes, such as the insertion, deletion or duplication of one or more nucleotides.
6. a “splice site” mutation, which is a mutation that results in the insertion, deletion or substitution of a nucleotide at the site of splicing.

In one embodiment, the mutation is a STOP codon mutation. For example, in one embodiment, the mutation is selected from at least one of the following:
A Q to STOP (TAG) at position 258 of SEQ ID NO: 1 or a homologous (corresponding) position in a homologous sequence;
A Q to STOP (TAA) at position 550 of SEQ ID NO: 1 or a homologous (corresponding) position in a homologous sequence;
A Q to STOP (TAA) at position 727 of SEQ ID NO: 2 or a homologous (or corresponding—such terms may be used interchangeably) position in a homologous sequence;
A Q to STOP (TAG) at position 360 of SEQ ID NO: 2 or a homologous (corresponding) position in a homologous sequence;
A Q to STOP (TAG) at position 258 of SEQ ID NO: 3 or a homologous (corresponding) position in a homologous sequence;
A Q to STOP (TAA) at position 482 of SEQ ID NO: 3 or a homologous (corresponding) position in a homologous sequence;

In a further embodiment, the method comprises introducing one or more mutations in the MRC nucleic acid sequence, which results in an amino acid mutation at one or more of the following positions, and biases the distribution of granule size in a population towards the small or smaller sizes (as described above):
Position 258 of SEQ ID NO: 1 or a homologous position in a homologous sequence. Preferably said mutation results in the termination of the protein at position 258 of SEQ ID NO: 1 or a homologous position in a homologous sequence. Preferably said mutation in the amino acid sequence arises from a mutation of one or more nucleotides in the nucleic acid sequence, wherein the mutation is at positions 1263 to 1265 of SEQ ID NO: 4 or a homologous position in a homologous sequence. Even more preferably, the mutation is a CAG to TAG mutation at positions 1263 to 1265 of SEQ ID NO: 4 or a homologous position in a homologous sequence. As used herein, this mutation may be referred to herein as K3272; and/or
Position 289 of SEQ ID NO: 1 or a homologous position in a homologous sequence. Preferably said mutation results in a substitution at position 289 of SEQ ID NO: 1 or a homologous position in a homologous sequence. In one embodiment, the homologous position is 288 in SEQ ID NO: 18 (maize), 287 in SEQ ID NO: 19 (rice) and position 285 in SEQ ID NO: 60 (potato). Even more preferably, the mutation is a L to F substitution. Preferably, said mutation in the amino acid sequence arises from a mutation of one or more nucleotides in the nucleic acid sequence, wherein the mutation is at positions 1356 to 1358 of SEQ ID NO: 4 or a homologous position in a homologous sequence. Even more preferably, the mutation is a CTT to TTT mutation at positions 1356 to 1358 of SEQ ID NO: 4 or a homologous position in a homologous sequence. As used herein, this mutation may be referred to herein as K598; and/or
Position 550 of SEQ ID NO: 1 or a homologous position in a homologous sequence. Preferably said mutation results in the termination of the protein at position 550 of SEQ ID NO: 1 or a homologous position in a homologous sequence. Preferably said mutation in the amino acid sequence arises from a mutation of one or more nucleotides in the nucleic acid sequence, wherein the mutation is at positions 2139 to 2141 of SEQ ID NO: 4 or a homologous position in a homologous sequence. Even more preferably, the mutation is a CAA to TAA mutation at positions 2139 to 2141 of SEQ ID NO: 4 or a homologous position in a homologous sequence. As used herein, this mutation may be referred to herein as K4681; and/or
Position 226 of SEQ ID NO: 29 or a homologous position in a homologous sequence. Preferably said mutation results in the termination of the protein at position 226 of SEQ ID NO: 29 or a homologous position in a homologous sequence. Preferably said mutation in the amino acid sequence arises from a mutation of one or more nucleotides in the nucleic acid sequence, wherein the mutation is at positions 682 to 684 of SEQ ID NO: 31 or a homologous position in a homologous sequence. Even more preferably, the mutation is a CAG to TAG mutation at positions 682 to 684 of SEQ ID NO: 31 or a homologous position in a homologous sequence. As used herein, this mutation may be referred to herein as K3078; and/or
Position 26 of SEQ ID NO: 29 or a homologous position in a homologous sequence. Preferably said mutation results in the termination of the protein at position 26 of SEQ ID NO: 29 or a homologous position in a homologous sequence. Preferably said mutation in the amino acid sequence arises from a mutation of one or more nucleotides in the nucleic acid sequence, wherein the mutation is at positions 82 to 84 of SEQ ID NO: 31 or a homologous position in a homologous sequence. Even more preferably, the mutation is a TGG to TGA mutation at positions 82 to 84 of SEQ ID NO: 31 or a homologous position in a homologous sequence. As used herein, this mutation may be referred to herein as K4305.

In one embodiment, the method may comprise introducing one or more of the K372, K598 and/or K4681 mutations (or homologous mutations) as described herein into a MRC nucleic acid. In a further embodiment, the method may comprise introducing the following mutations.
a K3272 and a K3078 mutation (or homologous mutations) into a MRC nucleic acid. A plant comprising both mutations is described herein as mrc-1; or
a K4681 and a K4305 mutation (or homologous mutations) into a MRC nucleic acid. A plant comprising both mutations is described herein as mrc-2; or
a K598 (or homologous mutations) into a MRC nucleic acid. A plant comprising both mutations is described herein as mrc-3.

In an alternative embodiment, the one or more mutations in the MRC nucleic acid sequence results in an amino acid mutation at one or more of the following positions, and biases the distribution of granule size in a population towards the small or smaller sizes (as described above):
Position 727 of SEQ ID NO: 2 or a homologous position in a homologous sequence. Preferably said mutation results in the termination of the protein at position 727 of SEQ ID NO: 2 or a homologous position in a homologous sequence. Preferably said mutation in the amino acid sequence arises from a mutation of one or more nucleotides in the nucleic acid sequence, wherein the mutation is at positions 2670 to 2672 of SEQ ID NO: 5 or a homologous position in a homologous sequence. Even more preferably, the mutation is a CAA to TAA mutation at positions 2670 to 2672 of SEQ ID NO: 5 or a homologous position in a homologous sequence. As used herein, this mutation may be referred to herein as Cadenza0199; and/or
Position 360 of SEQ ID NO: 2 or a homologous position in a homologous sequence. Preferably said mutation results in the termination of the protein at position 360 of SEQ ID NO: 2 or a homologous position in a homologous sequence. Preferably said mutation in the amino acid sequence arises from a mutation of one or more nucleotides in the nucleic acid sequence, wherein the mutation is at positions 1569 to 1571 of SEQ ID NO: 5 or a homologous position in a homologous sequence. Even more preferably, the mutation is a CAG to TAG mutation at positions 1569 to 1571 of SEQ ID NO: 5 or a homologous position in a homologous sequence. As used herein, this mutation may be referred to herein as Cadenza0377; and/or
Position 221 of SEQ ID NO: 30 or a homologous position in a homologous sequence. Preferably said mutation results in an amino acid substitution, preferably a E to K substitution at position 221 of SEQ ID NO: 30 or a homologous position in a homologous sequence. Preferably said mutation in the amino acid sequence arises from a mutation of one or more nucleotides in the nucleic acid sequence, wherein the mutation is at positions 667 to 669 of SEQ ID NO: 32 or a homologous position in a homologous sequence. Even more preferably, the mutation is a GAG to AAG mutation at positions 667 to 669 of SEQ ID NO: 32 or a homologous position in a homologous sequence. As used herein, this mutation may be referred to herein as Cadenza1715; and/or
Position 258 of SEQ ID NO: 3 or a homologous position in a homologous sequence. Preferably said mutation results in the termination of the protein at position 258 of SEQ ID NO: 3 or a homologous position in a homologous sequence. Preferably said mutation in the amino acid sequence arises from a mutation of one or more nucleotides in the nucleic acid sequence, wherein the mutation is at positions 1353 to 1355 of SEQ ID NO: 6 or a homologous position in a homologous sequence. Even more preferably, the mutation is a CAG to TAG mutation at positions 1353 to 1355 of SEQ ID NO: 6 or a homologous position in a homologous sequence. As used herein, this mutation may be referred to herein as Cadenza1012; and/or
Position 482 of SEQ ID NO: 3 or a homologous position in a homologous sequence. Preferably said mutation results in the termination of the protein at position 482 of SEQ ID NO: 3 or a homologous position in a homologous sequence. Preferably said mutation in the amino acid sequence arises from a mutation of one or more nucleotides in the nucleic acid sequence, wherein the mutation is at positions 2025 to 2027 of SEQ ID NO: 6 or a homologous position in a homologous sequence. Even more preferably, the mutation is a CAA to TAA mutation at positions 2025 to 2027 of SEQ ID NO: 6 or a homologous position in a homologous sequence. As used herein, this mutation may be referred to herein as Cadenza1092.

In one embodiment, the method may comprise introducing one or more of the Cadenza 0199, 0377, 1715, 1012 and/or 1092 mutations (or homologous mutations) as described herein into a MRC nucleic acid. In a further embodiment, the method may comprise introducing the following mutations:
a Cadenza 0199, 1715 and 1012 mutation (or homologous mutations) into a MRC nucleic acid. A plant comprising both mutations is described herein as mrc-4; or
a Cadenza 0199, 1715 and 1092 mutation (or homologous mutations) into a MRC nucleic acid. A plant comprising both mutations is described herein as mrc-5; or
a Cadenza 0377, 1715 and 1012 mutation (or homologous mutations) into a MRC nucleic acid. A plant comprising both mutations is described herein as mrc-6; or
a Cadenza 0377, 1715 and 1092 mutation (or homologous mutations) into a MRC nucleic acid. A plant comprising both mutations is described herein as mrc-7.

In an alternative embodiment, the one or more mutations in the MRC nucleic acid sequence results in an amino acid mutation at one or more of the following positions, and biases the distribution of granule size in a population towards the larger sizes (as described above):
Position 394 of SEQ ID NO: 1 or a homologous position in a homologous sequence. Preferably said mutation results in an amino acid substitution, preferably an L to F substitution at position 394 of SEQ ID NO: 1 or a homologous position in a homologous sequence. Preferably said mutation in the amino acid sequence arises from a mutation of one or more nucleotides in the nucleic acid sequence, wherein the mutation is at positions 1671 to 1673 of SEQ ID NO: 4 or a homologous position in a homologous sequence. Even more preferably, the mutation is a CTC to TTC mutation at positions 1671 to 1673 of SEQ ID NO: 4 or a homologous position in a homologous sequence. As used herein, this mutation may be referred to herein as Kronos775;
Position 681 of SEQ ID NO: 1 or a homologous position in a homologous sequence. Preferably said mutation results in an amino acid substitution, preferably a P to S substitution at position 681 of SEQ ID NO: 1 or a homologous position in a homologous sequence. Preferably said mutation in the amino acid sequence arises from a mutation of one or more nucleotides in the nucleic acid sequence, wherein the mutation is at positions 2532 to 2534 of SEQ ID NO: 4 or a homologous position in a homologous sequence. Even more preferably, the mutation is a CCA to TCA mutation at positions 2532 to 2534 of SEQ ID NO: 4 or a homologous position in a homologous sequence. As used herein, this mutation may be referred to herein as Kronos2096; and/or
Position 625 of SEQ ID NO: 1 or a homologous position in a homologous sequence. Preferably said mutation results in an amino acid substitution, preferably an A to T substitution at position 625 of SEQ ID NO: 1 or a homologous position in a homologous sequence. Preferably said mutation in the amino acid sequence arises from a mutation of one or more nucleotides in the nucleic acid sequence, wherein the mutation is at positions 2364 to 2366 of SEQ ID NO: 4 or a homologous position in a homologous sequence. Even more preferably, the mutation is a GCA to ACA mutation at positions 2364 to 2366 of SEQ ID NO: 4 or a homologous position in a homologous sequence. As used herein, this mutation may be referred to herein as Kronos2485.

In general, the skilled person will understand that at least one mutation as defined above and which leads to the insertion, deletion or substitution of at least one nucleic acid or amino acid compared to the wild-type MRC promoter or MRC nucleic acid or protein sequence can affect the biological activity of the MRC protein.

In another embodiment, the method comprises introducing at least one mutation into the plant genome, where the mutation is the insertion of at least one additional copy of a nucleic acid sequence encoding at least one MRC polypeptide such that the nucleic acid is operably linked to a regulatory sequence, and wherein the wherein the mutation is introduced using targeted genome editing. Alternatively, the method comprises introducing one or more mutations into the promoter of a MRC nucleic acid. Preferably, the mutation is introduced using ZFNs, TALENs or CRISPR/Cas9. In one embodiment, the MRC polypeptide sequence is selected from one of SEQ ID Nos 1 2 and/or 3. In a further embodiment, the nucleic acid sequence is selected from SEQ ID Nos 4, 5, 6, 7, 8, 9, 31, 32, 33, 34 and/or 35 or a functional variant or homolog thereof. A functional variant or homolog is defined herein.

In one embodiment a mutation may be introduced into the MRC promoter and at least one mutation is introduced into the MRC gene.

The skilled person would understand that suitable homologues and the homologous positions in these sequences can be identified by sequence comparisons (e.g. BLAST, alignments) and identifications of conserved domains. Phylogenetic tree analysis using nucleotide or amino acid sequences can be used to establish orthology to MRC. There are predictors in the art that can be used to identify such sequences. The function of the homologue can be identified as described herein and a skilled person would thus be able to confirm the function. Homologous positions can thus be determined by performing sequence alignments once the homologous sequence has been identified. For example, homologues can be identified using a BLAST search of the plant genome of interest using the wheat (Kronos or Cadenza) MRC as a query (i.e. one of the sequences defined in SEQ ID NOs: 1 to 9).

In one embodiment, the mutation is introduced using mutagenesis or targeted genome editing. That is, in one embodiment, the invention relates to a method and plant that has been generated by genetic engineering methods as described above, and does not encompass naturally occurring varieties.

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customisable DNA binding proteins can be used: meganucleases derived from microbial mobile genetic elements, ZF nucleases based on eukaryotic transcription factors, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of FokI to direct nucleolytic activity toward specific genomic loci.

Upon delivery into host cells via the bacterial type II secretion system, TAL effectors enter the nucleus, bind to effector-specific sequences in host gene promoters and activate transcription. Their targeting specificity is determined by a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat-variable di-residue (RVD). The RVD that determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the FokI nuclease to create a TAL effector nuclease (TALEN) which makes targeted DNA double-strand breaks (DSBs) in vivo for genome editing. The use of this technology in genome editing is well described in the art, for example in U.S. Pat. Nos. 8,440,431, 8,440,432 and 8,450,471. Cermak T et al. describes a set of customized plasmids that can be used with the Golden Gate cloning method to assemble multiple DNA fragments. As described therein, the Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct and involves two steps: (i) assembly of repeat modules into intermediary arrays of 1-10 repeats and (ii) joining of the intermediary arrays into a backbone to make the final construct. Accordingly, using techniques known in the art it is possible to design a TAL effector that targets a MRC gene or promoter sequence as described herein.

Another genome editing method that can be used according to the various aspects of the invention is CRISPR. The use of this technology in genome editing is well described in the art, for example in U.S. Pat. No. 8,697,359 and references cited herein. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

One major advantage of the CRISPR-Cas9 system, as compared to conventional gene targeting and other programmable endonucleases is the ease of multiplexing, where multiple genes can be mutated simultaneously simply by using multiple sgRNAs each targeting a different gene. In addition, where two sgRNAs are used flanking a genomic region, the intervening section can be deleted or inverted (Wiles et al., 2015).

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3. Accordingly, using techniques known in the art it is possible to design sgRNA molecules that targets a MRC gene or promoter sequence as described herein. In one embodiment, the method comprises using any of the nucleic acid constructs or sgRNA molecules described herein.

Cas9 expression plasmids for use in the methods of the invention can be constructed as described in the art.

In one embodiment, the method uses a sgRNA to introduce a targeted SNP or mutation, in particular one of the substitutions described herein, into a MRC gene. As explained herein, the introduction of a template DNA strand, following a sgRNA-mediated snip in the double-stranded DNA, can be used to produce a specific targeted mutation (i.e. a SNP) in the gene using homology directed repair. In an alternative embodiment, at least one mutation may be introduced into the MRC gene and/or promoter, particularly at the positions described above, using any CRISPR technique known to the skilled person. In another example, sgRNA (for example, as described herein) can be used with a modified Cas9 protein, such as nickase Cas9 or nCas9 or a “dead” Cas9 (dCas9) fused to a “Base Editor”—such as an enzyme, for example a deaminase such as cytidine deaminase, or TadA (tRNA adenosine deaminase) or ADAR or APOBEC. These enzymes are able to substitute one base for another. As a result no DNA is deleted, but a single substitution is made.

Alternatively, more conventional mutagenesis methods can be used to introduce at least one mutation into a MRC gene or MRC promoter sequence. These methods include both physical and chemical mutagenesis. A skilled person will know further approaches can be used to generate such mutants, and methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein.

In one embodiment, insertional mutagenesis is used, for example using T-DNA mutagenesis (which inserts pieces of the T-DNA from the Agrobacterium tumefaciens Ti-Plasmid into DNA causing either loss of gene function or gain of gene function mutations), site-directed nucleases (SDNs) or transposons as a mutagen. Insertional mutagenesis is an alternative means of disrupting gene function and is based on the insertion of foreign DNA into the gene of interest (see Krysan et al, The Plant Cell, Vol. 11, 2283-2290, December 1999). Accordingly, in one embodiment, T-DNA is used as an insertional mutagen to disrupt MRC gene or MRC promoter expression. T-DNA not only disrupts the expression of the gene into which it is inserted, but also acts as a marker for subsequent identification of the mutation. Since the sequence of the inserted element is known, the gene in which the insertion has occurred can be recovered, using various cloning or PCR-based strategies. The insertion of a piece of T-DNA in the order of 5 to 25 kb in length generally produces a disruption of gene function. If a large enough population of T-DNA transformed lines is generated, there are reasonably good chances of finding a transgenic plant carrying a T-DNA insert within any gene of interest. Transformation of spores with T-DNA is achieved by an Agrobacterium-mediated method which involves exposing plant cells and tissues to a suspension of Agrobacterium cells.

The details of this method are well known to a skilled person. In short, plant transformation by Agrobacterium results in the integration into the nuclear genome of a sequence called T-DNA, which is carried on a bacterial plasmid. The use of T-DNA transformation leads to stable single insertions. Further mutant analysis of the resultant transformed lines is straightforward and each individual insertion line can be rapidly characterized by direct sequencing and analysis of DNA flanking the insertion. Gene expression in the mutant is compared to expression of the MRC nucleic acid sequence in a wild type plant and phenotypic analysis is also carried out.

In another embodiment, mutagenesis is physical mutagenesis, such as application of ultraviolet radiation, X-rays, gamma rays, fast or thermal neutrons or protons. The targeted population can then be screened to identify an MRC mutant with reduced expression or activity.

In another embodiment of the various aspects of the invention, the method comprises mutagenizing a plant population with a mutagen. The mutagen may be a fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde. Again, the targeted population can then be screened to identify a MRC gene or promoter mutant.

In another embodiment, the method used to create and analyse mutations is targeting induced local lesions in genomes (TILLING), reviewed in Henikoff et al, 2004. In this method, seeds are mutagenised with a chemical mutagen, for example EMS. The resulting M1 plants are self-fertilised and the M2 generation of individuals is used to prepare DNA samples for mutational screening. DNA samples are pooled and arrayed on microtiter plates and subjected to gene specific PCR. The PCR amplification products may be screened for mutations in the MRC target gene using any method that identifies heteroduplexes between wild type and mutant genes. For example, but not limited to, denaturing high pressure liquid chromatography (dHPLC), constant denaturant capillary electrophoresis (CDCE), temperature gradient capillary electrophoresis (TGCE), or by fragmentation using chemical cleavage. Preferably the PCR amplification products are incubated with an endonuclease that preferentially cleaves mismatches in heteroduplexes between wild type and mutant sequences. Cleavage products are electrophoresed using an automated sequencing gel apparatus, and gel images are analyzed with the aid of a standard commercial image-processing program. Any primer specific to the MRC nucleic acid sequence may be utilized to amplify the MRC nucleic acid sequence within the pooled DNA sample. Preferably, the primer is designed to amplify the regions of the MRC gene where useful mutations are most likely to arise, specifically in the areas of the MRC gene that are highly conserved and/or confer activity as explained elsewhere. To facilitate detection of PCR products on a gel, the PCR primer may be labelled using any conventional labelling method. In an alternative embodiment, the method used to create and analyse mutations is EcoTILLING. EcoTILLING is molecular technique that is similar to TILLING, except that its objective is to uncover natural variation in a given population as opposed to induced mutations. The first publication of the EcoTILLING method was described in Comai et al. 2004.

Rapid high-throughput screening procedures thus allow the analysis of amplification products for identifying a mutation conferring the reduction or inactivation of the expression of the MRC gene as compared to a corresponding non-mutagenised wild type plant. Once a mutation is identified in a gene of interest, the seeds of the M2 plant carrying that mutation are grown into adult M3 plants and screened for the phenotypic characteristics associated with the target gene MRC. Loss of and reduced function mutants with an altered starch granule size, number and/or distribution compared to a control can thus be identified.

Plants obtained or obtainable by such method which carry a functional mutation in the endogenous MRC gene or promoter locus are also within the scope of the invention.

In an alternative embodiment, the expression of the MRC gene may be reduced at either the level of transcription or translation. For example, expression of a MRC nucleic acid or MRC promoter sequence, as defined herein, can be reduced or silenced using a number of gene silencing methods known to the skilled person, such as, but not limited to, the use of small interfering nucleic acids (siNA) against MRC. “Gene silencing” is a term generally used to refer to suppression of expression of a gene via sequence-specific interactions that are mediated by RNA molecules. The degree of reduction may be so as to totally abolish production of the encoded gene product, but more usually the abolition of expression is partial, with some degree of expression remaining. The term should not therefore be taken to require complete “silencing” of expression.

In one embodiment, the siNA may include, short interfering RNA (siRNA), double-stranded RNA (dsRNA), micro-RNA (miRNA), antagomirs and short hairpin RNA (shRNA) capable of mediating RNA interference.

The inhibition of expression and/or activity can be measured by determining the presence and/or amount of MRC transcript using techniques well known to the skilled person (such as Northern Blotting, RT-PCR and so on).

Transgenes may be used to suppress endogenous plant genes. This was discovered originally when chalcone synthase transgenes in petunia caused suppression of the endogenous chalcone synthase genes and indicated by easily visible pigmentation changes. Subsequently it has been described how many, if not all plant genes can be “silenced” by transgenes. Gene silencing requires sequence similarity between the transgene and the gene that becomes silenced. This sequence homology may involve promoter regions or coding regions of the silenced target gene. When coding regions are involved, the transgene able to cause gene silencing may have been constructed with a promoter that would transcribe either the sense or the antisense orientation of the coding sequence RNA. It is likely that the various examples of gene silencing involve different mechanisms that are not well understood. In different examples there may be transcriptional or post-transcriptional gene silencing and both may be used according to the methods of the invention.

The mechanisms of gene silencing and their application in genetic engineering, which were first discovered in plants in the early 1990s and then shown in Caenorhabditis elegans are extensively described in the literature.

RNA-mediated gene suppression or RNA silencing according to the methods of the invention includes co-suppression wherein over-expression of the target sense RNA or mRNA, that is the MRC sense RNA or mRNA, leads to a reduction in the level of expression of the genes concerned. RNAs of the transgene and homologous endogenous gene are co-ordinately suppressed. Other techniques used in the methods of the invention include antisense RNA to reduce transcript levels of the endogenous target gene in a plant. In this method, RNA silencing does not affect the transcription of a gene locus, but only causes sequence-specific degradation of target mRNAs. An “antisense” nucleic acid sequence comprises a nucleotide sequence that is complementary to a “sense” nucleic acid sequence encoding a MRC protein, or a part of the protein, i.e. complementary to the coding strand of a double-stranded cDNA molecule or complementary to an mRNA transcript sequence. The antisense nucleic acid sequence is preferably complementary to the endogenous MRC gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire MRC nucleic acid sequence as defined herein, but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine-substituted nucleotides may be used. Examples of modified nucleotides that may be used to generate the antisense nucleic acid sequences are well known in the art. The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention hybridize with or bind to mRNA transcripts and/or insert into genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using vectors.

RNA interference (RNAi) is another post-transcriptional gene-silencing phenomenon which may be used according to the methods of the invention. This is induced by double-stranded RNA in which mRNA that is homologous to the dsRNA is specifically degraded. It refers to the process of sequence-specific post-transcriptional gene silencing mediated by short interfering RNAs (siRNA). The process of RNAi begins when the enzyme, DICER, encounters dsRNA and chops it into pieces called small-interfering RNAs (siRNA). This enzyme belongs to the RNase III nuclease family. A complex of proteins gathers up these RNA remains and uses their code as a guide to search out and destroy any RNAs in the cell with a matching sequence, such as target mRNA.

Artificial and/or natural microRNAs (miRNAs) may be used to knock out gene expression and/or mRNA translation. MicroRNAs (miRNAs) miRNAs are typically single stranded small RNAs typically 19-24 nucleotides long. Most plant miRNAs have perfect or near-perfect complementarity with their target sequences. However, there are natural targets with up to five mismatches. They are processed from longer non-coding RNAs with characteristic fold-back structures by double-strand specific RNases of the Dicer family.

Upon processing, they are incorporated in the RNA-induced silencing complex (RISC) by binding to its main component, an Argonaute protein. miRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes. Artificial microRNA (amiRNA) technology has been applied in Arabidopsis thaliana and other plants to efficiently silence target genes of interest. The design principles for amiRNAs have been generalized and integrated into a web-based tool (http://wmd.weigelworld.org).

Thus, according to the various aspects of the invention a plant may be transformed to introduce a RNAi, shRNA, snRNA, dsRNA, siRNA, miRNA, ta-siRNA, amiRNA or cosuppression molecule that has been designed to target the expression of an MRC nucleic acid sequence and selectively decreases or inhibits the expression of the gene or stability of its transcript. Preferably, the RNAi, snRNA, dsRNA, shRNA siRNA, miRNA, amiRNA, ta-siRNA or cosuppression molecule used according to the various aspects of the invention comprises a fragment of at least 17 nt, preferably 22 to 26 nt and can be designed on the basis of the information shown in any of SEQ ID NOs: 4 to 9, 11, 12, 14, 15, 17, 18, 20, 21, 22 to 28 and 31 to 37. Guidelines for designing effective siRNAs are known to the skilled person. Briefly, a short fragment of the target gene sequence (e.g., 19-40 nucleotides in length) is chosen as the target sequence of the siRNA of the invention. The short fragment of target gene sequence is a fragment of the target gene mRNA. In preferred embodiments, the criteria for choosing a sequence fragment from the target gene mRNA to be a candidate siRNA molecule include 1) a sequence from the target gene mRNA that is at least 50-100 nucleotides from the 5′ or 3′ end of the native mRNA molecule, 2) a sequence from the target gene mRNA that has a G/C content of between 30% and 70%, most preferably around 50%, 3) a sequence from the target gene mRNA that does not contain repetitive sequences (e.g., AAA, CCC, GGG, TTT, AAAA, CCCC, GGGG, TTTT), 4) a sequence from the target gene mRNA that is accessible in the mRNA, 5) a sequence from the target gene mRNA that is unique to the target gene, 6) avoids regions within 75 bases of a start codon. The sequence fragment from the target gene mRNA may meet one or more of the criteria identified above. The selected gene is introduced as a nucleotide sequence in a prediction program that takes into account all the variables described above for the design of optimal oligonucleotides. This program scans any mRNA nucleotide sequence for regions susceptible to be targeted by siRNAs. The output of this analysis is a score of possible siRNA oligonucleotides. The highest scores are used to design double stranded RNA oligonucleotides that are typically made by chemical synthesis. In addition to siRNA which is complementary to the mRNA target region, degenerate siRNA sequences may be used to target homologous regions. siRNAs according to the invention can be synthesized by any method known in the art. RNAs are preferably chemically synthesized using appropriately protected ribonucleoside phosphoramidites and a conventional DNA/RNA synthesizer. Additionally, siRNAs can be obtained from commercial RNA oligonucleotide synthesis suppliers.

siRNA molecules according to the aspects of the invention may be double stranded. In one embodiment, double stranded siRNA molecules comprise blunt ends. In another embodiment, double stranded siRNA molecules comprise overhanging nucleotides (e.g., 1-5 nucleotide overhangs, preferably 2 nucleotide overhangs). In some embodiments, the siRNA is a short hairpin RNA (shRNA); and the two strands of the siRNA molecule may be connected by a linker region (e.g., a nucleotide linker or a non-nucleotide linker). The siRNAs of the invention may contain one or more modified nucleotides and/or non-phosphodiester linkages. Chemical modifications well known in the art are capable of increasing stability, availability, and/or cell uptake of the siRNA. The skilled person will be aware of other types of chemical modification which may be incorporated into RNA molecules.

In one embodiment, recombinant DNA constructs as described in U.S. Pat. No. 6,635,805, incorporated herein by reference, may be used.

The silencing RNA molecule is introduced into the plant using conventional methods, for example a vector and Agrobacterium-mediated transformation. Stably transformed plants are generated and expression of the MRC gene compared to a wild type control plant is analysed.

Silencing or reducing expression levels of MRC nucleic acid may also be achieved using virus-induced gene silencing.

Thus, in one embodiment of the invention, the plant expresses a nucleic acid construct comprising a RNAi, shRNA snRNA, dsRNA, siRNA, miRNA, ta-siRNA, amiRNA or co-suppression molecule that targets the MRC nucleic acid sequence as described herein and reduces expression of the endogenous MRC nucleic acid sequence. A gene is targeted when, for example, the RNAi, snRNA, dsRNA, siRNA, shRNA miRNA, ta-siRNA, amiRNA or cosuppression molecule selectively decreases or inhibits the expression of the gene compared to a control plant. Alternatively, a RNAi, snRNA, dsRNA, siRNA, miRNA, ta-siRNA, amiRNA or cosuppression molecule targets a MRC nucleic acid sequence when the RNAi, shRNA snRNA, dsRNA, siRNA, miRNA, ta-siRNA, amiRNA or co-suppression molecule hybridises under stringent conditions to the gene transcript.

A further approach to gene silencing is by targeting nucleic acid sequences complementary to the regulatory region of the gene (e.g., the promoter and/or enhancers) of MRC to form triple helical structures that prevent transcription of the gene in target cells. Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved.

In one embodiment, the suppressor nucleic acids may be anti-sense suppressors of expression of the MRC polypeptides. In using anti-sense sequences to down-regulate gene expression, a nucleotide sequence is placed under the control of a promoter in a “reverse orientation” such that transcription yields RNA which is complementary to normal mRNA transcribed from the “sense” strand of the target gene.

An anti-sense suppressor nucleic acid may comprise an anti-sense sequence of at least 10 nucleotides from the target nucleotide sequence. It may be preferable that there is complete sequence identity in the sequence used for down-regulation of expression of a target sequence, and the target sequence, although total complementarity or similarity of sequence is not essential. One or more nucleotides may differ in the sequence used from the target gene. Thus, a sequence employed in a down-regulation of gene expression in accordance with the present invention may be a wild-type sequence (e.g. gene) selected from those available, or a variant of such a sequence.

The sequence need not include an open reading frame or specify an RNA that would be translatable. It may be preferred for there to be sufficient homology for the respective anti-sense and sense RNA molecules to hybridise. There may be down regulation of gene expression even where there is about 5%, 10%, 15% or 20% or more mismatch between the sequence used and the target gene. Effectively, the homology should be sufficient for the down-regulation of gene expression to take place.

Suppressor nucleic acids may be operably linked to tissue-specific or inducible promoters. For example, seed and endosperm-specific promoters can be used to specifically down-regulate an MRC nucleic acid in developing seeds to alter granule size in that organ specifically.

Nucleic acid which suppresses expression of an MRC polypeptide as described herein may be operably linked to a heterologous regulatory-sequence, such as a promoter, for example a constitutive, inducible, tissue-specific or developmental specific promoter. The construct or vector may be transformed into plant cells and expressed as described herein. Plant cells comprising such vectors are also within the scope of the invention.

In another aspect, the invention relates to a silencing construct obtainable or obtained by a method as described herein and to a plant cell comprising such construct.

Thus, aspects of the invention involve targeted mutagenesis methods, specifically genome editing, and in a preferred embodiment exclude embodiments that are solely based on generating plants by traditional breeding methods.

In another embodiment, the method of increasing the expression of a MRC nucleic acid comprises introducing and expressing a nucleic acid construct comprising a nucleic acid encoding a MRC polypeptide operably linked to a regulatory sequence wherein the at least one MRC polypeptide is selected from SEQ ID Nos 1, 2, 3, 29 or 30 or a functional variant or homolog as defined herein. In one embodiment, the nucleic acid sequence encodes at least one MRC polypeptide selected from SEQ ID Nos 1 or 2 or 2 or 2 and 3. In a further embodiment, the nucleic acid sequence is selected from SEQ ID Nos 4, 5, 6, 7, 8, 9, 31, 32, 33, 34 and 35 or a functional variant or homolog thereof. A functional variant or homolog is defined herein. In one embodiment, the regulatory sequence is a promoter. In one embodiment, the promoter is a constitutive promoter such as 35S or the Ubiquitin promoter. In another embodiment, the promoter is a tissue-specific promoter such as the HMW glutenin promoter.

In another aspect, the invention extends to a plant obtained or obtainable by a method as described herein.

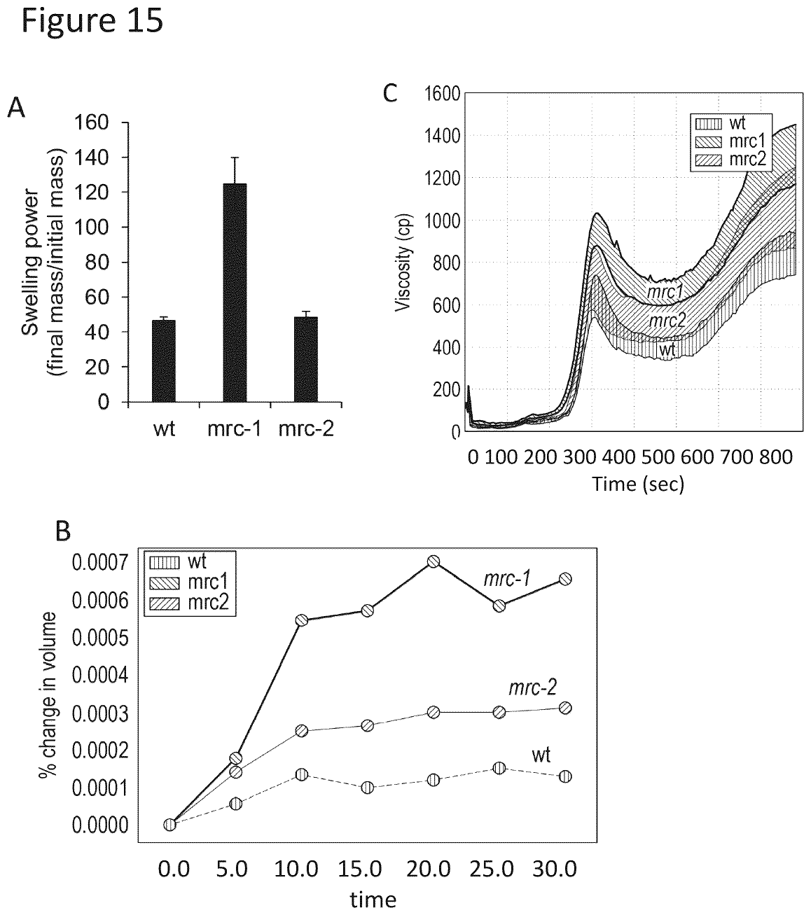

In a further aspect of the invention, there is provided a method of altering a physiochemical property of starch, the method comprising altering the expression of at least one MRC (MYOSIN-RESMBLING CHLOROPLAST PROTEIN) nucleic acid and/or altering the activity of a MRC polypeptide as described above. As shown in FIG. 15, in one embodiment, the physiochemical property is selected from gelatinisation temperature, swelling power and viscosity. In a preferred embodiment, the method increases or decreases at least one of starch digestion rate, gelatinisation temperature, swelling power and viscosity. More preferably, the method comprises increasing the onset and gelatinisation temperature. An increase as used herein may be at least 1%, 2%, 3%, 4%, 5%, 6%, 7%, 8%, 9%, 10% 11%, 12%, 13%, 14%, 15%, 16%, 17%, 18%, 19%, 20%, 30%, 40% or 50% in comparison to a control plant.

In another aspect of the invention, there is provided a method of altering a plant with a bimodal size distribution of starch granules towards a unimodal distribution of starch granules, the method comprising reducing or abolishing the expression of at least one MRC nucleic acid and/or reducing or abolishing the activity of a MRC polypeptide as described above.

In a further aspect of the invention, there is provided a method of altering the initiation of granule formation in a starch storage organ of a plant, the method comprising alerting the expression of at least one MRC nucleic acid and/or altering the activity of at least one MRC polypeptide as described above.

In another aspect of the invention there is provided a method of altering at least one of starch granule size, number and distribution in a plant, the method comprising reducing or abolishing the expression of at least one MRC nucleic acid and/or reducing or abolishing the activity of a MRC polypeptide (using any of the methods described herein), wherein the plant is not Arabidopsis. In one embodiment, the plant has a bimodal size distribution of starch granules, such as wheat, rye and barley, wherein preferably said bimodal distribution comprises a first population of starch granules of a first size or size range and a second population of starch granules of a second size or size range. More preferably, the method comprises decreasing the size of starch granules and/or increasing the number of starch granules in the first and/or second population of starch granules. In one embodiment, the method comprises decreasing the size of granules in the first population and/or increasing the number of granules in the second population. Alternatively, the method comprises increasing the size of granules in the first population and/or decreasing the number of starch granules in the second population. An increase or decrease is described elsewhere.

In one embodiment, the first population of starch granules comprises granules with an average spherical diameter over 15 μm, and wherein the second population of starch granules comprises granules with an average spherical diameter between 1 and 15 μm. Also described herein, is a genetically altered plant, part thereof or plant cell characterised by reduced or abolished expression (as described herein) or at least one MRC nucleic acid and/or reduced or abolished activity of a MRC polypeptide, wherein the plant is not Arabidopsis. In one example, the plant has a bimodal distribution of starch granules.

### Genetically Altered or Modified Plants and Methods of Producing Such Plants

In another aspect of the invention there is provided a genetically altered plant, part thereof or plant cell characterised in that the expression of MRC is altered, the plant does not express a functional MRC protein or expresses a MRC protein with reduced or increased function and/or activity. In one embodiment, the plant is a reduction (knock down) or loss of function (knock out) mutant wherein the function of the MRC nucleic acid sequence is reduced or lost compared to a wild type or control plant. Preferably, the plant is a knock down and not a knock out, meaning that the plant has reduced levels of MRC expression or expresses a MRC protein with reduced function and/or activity. To this end, a mutation is introduced into either the MRC gene sequence or the corresponding promoter sequence which disrupts the transcription of the gene. Therefore, preferably said plant comprises at least one mutation in the promoter and/or at least one gene for MRC. In one embodiment the plant may comprise a mutation in both the promoter and the at least one gene for MRC.

In an alternative embodiment, the plant is a gain-of-function mutation where the function or activity of the MRC polypeptide is increased compared to the wild-type or control plant. Again, to this end, a mutation is introduced into either the MRC gene sequence or the corresponding promoter sequence to either increase the transcription of the gene or increase the activity of the polypeptide. Therefore, preferably said plant comprises at least one mutation in the promoter and/or at least one gene for MRC. In one embodiment the plant may comprise a mutation in both the promoter and the at least one gene for MRC.

In a further aspect of the invention, there is provided a plant, part thereof or plant cell characterised by an alteration in the size distribution of starch granules in a starch storage organ. A definition of an alteration in a granule size distribution is described above.

The plant may be produced by introducing a mutation, preferably a deletion, insertion or substitution into the MRC gene and/or promoter sequence by any of the above described methods. Preferably said mutation is introduced into a least one plant cell and a plant regenerated from the at least one mutated plant cell.

In one embodiment, the plant is characterised by a shift in the distribution of granule size towards the smaller granule size. In other words, the plant may be characterised by an increase in the number of smaller granules, compared to a wild-type or control plant. In a further embodiment, the plant is characterised by a decrease in the average granule size. In a further embodiment, the plant comprises at least one loss of function mutation in a MRC gene and/or promoter as described herein.

In an alternative embodiment, the plant is characterised by a shift in the distribution of granule size towards the larger granule size. In other words, the plant may be characterised by an increase in the size and/or number of larger granule sizes, compared to a wild-type or control plant. In a further embodiment, the plant is characterised by an increase in the average granule size. In a further embodiment, the plant comprises at least one gain of function mutation in a MRC gene and/or promoter as described herein.

Alternatively, the plant or plant cell may comprise a nucleic acid construct expressing an RNAi molecule targeting the MRC gene as described herein. In one embodiment, said construct is stably incorporated into the plant genome. These techniques also include gene targeting using vectors that target the gene of interest and which allows for integration of a transgene at a specific site. The targeting construct is engineered to recombine with the target gene, which is accomplished by incorporating sequences from the gene itself into the construct. Recombination then occurs in the region of that sequence within the gene, resulting in the insertion of a foreign sequence to disrupt the gene. With its sequence interrupted, the altered gene will be translated into a nonfunctional protein, if it is translated at all.

In another aspect of the invention, there is provided a nucleic acid construct comprising a nucleic acid sequence encoding at least one MRC polypeptide operably linked to a regulatory sequence, wherein the at least one MRC polypeptide is selected from SEQ ID Nos 1, 2, 3, 29 or 30 or a functional variant or homolog as defined herein. In one embodiment, the nucleic acid sequence encodes at least one MRC polypeptide selected from SEQ ID Nos 1 or 2 or 2 or 2 and 3. In a further embodiment, the nucleic acid sequence is selected from SEQ ID Nos 4, 5, 6, 7, 8, 9, 31, 32, 33, 34 and 35 or a functional variant or homolog thereof. A functional variant or homolog is defined herein.

In one embodiment, the regulatory sequence is a promoter. In one embodiment, the promoter is a constitutive promoter such as 35S or the Ubiquitin promoter. In another embodiment, the promoter is a tissue-specific promoter such as the HMW glutenin promoter.

In another aspect of the invention, there is also provided a transgenic plant expressing the above-described nucleic acid construct.

In a further aspect of the invention, there is provided a method of making a transgenic plant, the method comprising introducing and expressing in a plant or plant cell the above-described nucleic acid construct. Method for introducing (or transforming) a plant or plant cell are described above. The method may further comprise regenerating a transgenic plant from the plant or plant cell and obtaining progeny plant, wherein the progeny plant comprises in its genome a nucleic acid sequence encoding MRC as described herein, linked to a regulatory sequence, wherein the progeny plant also display an alteration in starch granule size distribution compared to a wild-type or control plant. Methods of regenerating a plant are described elsewhere herein.

In another aspect of the invention there is provided a method for producing a genetically altered plant as described herein. In one embodiment, the method comprises introducing at least one mutation into the MRC gene and/or MRC promoter of preferably at least one plant cell using any mutagenesis technique described herein. Preferably said method further comprising regenerating a plant from the mutated plant cell.

The method may further comprise selecting one or more mutated plants, preferably for further propagation. Preferably said selected plants comprise at least one mutation in the MRC gene and/or promoter sequence. Preferably said plants are characterised by an altered of MRC expression and/or a reduced or abolished level of MRC polypeptide activity. Expression and/or activity levels of MRC can be measured by any standard technique known to the skilled person. A reduction is as described herein.

The selected plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In a further aspect of the invention there is provided a plant obtained or obtainable by the above described methods.

For the purposes of the invention, a “genetically altered plant” or “mutant plant” is a plant that has been genetically altered compared to the naturally occurring wild type (WT) plant. In one embodiment, a mutant plant is a plant that has been altered compared to the naturally occurring wild type (WT) plant using a mutagenesis method, such as any of the mutagenesis methods described herein. In one embodiment, the mutagenesis method is targeted genome modification or genome editing. In one embodiment, the plant genome has been altered compared to wild type sequences using a mutagenesis method. Such plants have an altered phenotype as described herein, such as an altered starch granule size distribution. Therefore, in this example, altered starch granule size distribution is conferred by the presence of an altered plant genome, for example, a mutated endogenous MRC gene or MRC promoter sequence. In one embodiment, the endogenous promoter or gene sequence is specifically targeted using targeted genome modification and the presence of a mutated gene or promoter sequence is not conferred by the presence of transgenes expressed in the plant. In other words, the genetically altered plant can be described as transgene-free.

A plant according to the various aspects of the invention, including the transgenic plants, methods and uses described herein may be a monocot or a dicot plant. Preferably, the plant is a crop plant or a biofuel plant. By crop plant is meant any plant which is grown on a commercial scale for human or animal consumption or use. In a preferred embodiment, the plant is a cereal.

In a most preferred embodiment, the plant is selected from wheat, barley, rye, maize, potato, sorghum and rice. In a further embodiment, the plant is wheat.

The term “plant” as used herein encompasses whole plants, ancestors and progeny of the plants and plant parts, including seeds, fruit, shoots, stems, leaves, roots (including tubers), flowers, tissues and organs, wherein each of the aforementioned comprise the nucleic acid construct as described herein. The term “plant” also encompasses plant cells, suspension cultures, callus tissue, embryos, meristematic regions, gametophytes, sporophytes, pollen and microspores, again wherein each of the aforementioned comprises the nucleic acid construct as described herein.

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. Another product that may derived from the harvestable parts of the plant of the invention is biodiesel. The invention also relates to food products and food supplements comprising the plant of the invention or parts thereof. In one embodiment, the food products may be animal feed. In another aspect of the invention, there is provided a product derived from a plant as described herein or from a part thereof.

In a most preferred embodiment, the plant part or harvestable product is a seed or grain. Therefore, in a further aspect of the invention, there is provided a seed or grain produced from a genetically altered plant as described herein.

In another embodiment of the invention, the plant part is a starch storage organ comprising starch granules which have a mean granule size which is increased or decreased by 3% or more, 5% or more, 10% or more, 15% or more, 20% or more, 25% or more, 30% or more, 35% or more, 40% or more or 45% or more compared to the mean granule size in a control or wild-type plant. Preferably, the size is decreased. Alternatively the size is increased.

In a yet further embodiment, there is provided a starch storage organ comprising starch granules which have a granule size standard deviation which is increased or decreased by 3% or more, 5% or more, 10% or more, 15% or more, 20% or more, 25% or more, 30% or more, 35% or more, 40% or more or 45% or more compared to the starch granule size standard deviation in a control or wild-type plant. Preferably the standard deviation is reduced, e.g. narrowed.

In an alternative embodiment, the plant part is pollen, a propagule or progeny of the genetically altered plant described herein. Accordingly, in a further aspect of the invention there is provided pollen, a propagule or progeny of the genetically altered plant as described herein.

In another aspect of the invention, there is provided starch or a starch component obtained or obtainable from at least one plant cell of the genetically altered plant described herein or the grain of a genetically altered plant described herein.

There is also provided a food or feed composition prepared from the grain or starch or starch composition described herein.

Finally, there is also provided the use of the grain or starch described herein as a food or feedstuff, in biofuel (bioethanol) production or in any pharmaceutical, cosmetic or industrial application. Examples of industrial applications include the brewing, papermaking and plastic industries. Examples of food include bread, biscuits, baked goods based on wheat flour and pasta.

A control plant as used herein according to all of the aspects of the invention is a plant which has not been modified according to the methods of the invention. Accordingly, in one embodiment, the control plant does not have altered expression of a MRC nucleic acid and/or altered activity of a MRC polypeptide, as described herein. In an alternative embodiment, the plant been genetically modified, as described above. In one embodiment, the control plant is a wild type plant. The control plant is typically of the same plant species, preferably having the same genetic background as the modified plant.

Genome Editing Constructs for Use with the Methods for Targeted Genome Modification Described Herein

By “crRNA” or CRISPR RNA is meant the sequence of RNA that contains the protospacer element and additional nucleotides that are complementary to the tracrRNA.

By “tracrRNA” (transactivating RNA) is meant the sequence of RNA that hybridises to the crRNA and binds a CRISPR enzyme, such as Cas9 thereby activating the nuclease complex to introduce double-stranded breaks at specific sites within the genomic sequence of at least one MRC nucleic acid or promoter sequence.

By “protospacer element” is meant the portion of crRNA (or sgRNA) that is complementary to the genomic DNA target sequence, usually around 20 nucleotides in length. This may also be known as a spacer or targeting sequence.

By “sgRNA” (single-guide RNA) is meant the combination of tracrRNA and crRNA in a single RNA molecule, preferably also including a linker loop (that links the tracrRNA and crRNA into a single molecule). “sgRNA” may also be referred to as “gRNA” and in the present context, the terms are interchangeable. The sgRNA or gRNA provide both targeting specificity and scaffolding/binding ability for a Cas nuclease. A gRNA may refer to a dual RNA molecule comprising a crRNA molecule and a tracrRNA molecule.

By “TAL effector” (transcription activator-like (TAL) effector) or TALE is meant a protein sequence that can bind the genomic DNA target sequence (a sequence within the MRC gene or promoter sequence) and that can be fused to the cleavage domain of an endonuclease such as FokI to create TAL effector nucleases or TALENS or meganucleases to create megaTALs. A TALE protein is composed of a central domain that is responsible for DNA binding, a nuclear-localisation signal and a domain that activates target gene transcription. The DNA-binding domain consists of monomers and each monomer can bind one nucleotide in the target nucleotide sequence. Monomers are tandem repeats of 33-35 amino acids, of which the two amino acids located at positions 12 and 13 are highly variable (repeat variable diresidue, RVD). It is the RVDs that are responsible for the recognition of a single specific nucleotide. HD targets cytosine; NI targets adenine, NG targets thymine and NN targets guanine (although NN can also bind to adenine with lower specificity).

In another aspect of the invention there is provided a nucleic acid construct wherein the nucleic acid construct comprises a nucleic acid sequence that encodes at least one DNA-binding domain. In one embodiment, the DNA-binding domain can bind to a sequence in the MRC gene and/or promoter. Preferably said sequence is selected from one of SEQ ID NO: 39 to 42 and are target sequences in a MRC gene. In one embodiment, the nucleic acid construct comprises one or more DNA-binding domains, such that the construct can bind to one or more, preferably at least two or three sequences in the MRC gene. In one embodiment, the target sequences are selected from one of SEQ ID NO: 39 to 42.

In a further embodiment, said construct further comprises a nucleic acid encoding at least one sequence specific nuclease (SSN) such as FokI or a Cas protein.

In one embodiment, the nucleic acid construct encodes at least one protospacer element wherein the sequence of the protospacer element is selected from SEQ ID NO: 43 to 46 or a variant thereof. In one example, the nucleic acid construct may comprise one, two or three protospacer sequences, wherein the sequence of the protospacer sequences is selected from SEQ ID NO: 43 to 46.

In a further embodiment, the nucleic acid construct comprises a crRNA-encoding sequence. As defined above, a crRNA sequence may comprise the protospacer elements as defined above and preferably additional nucleotides that are complementary to the tracrRNA. An appropriate sequence for the additional nucleotides will be known to the skilled person as these are defined by the choice of Cas protein.

In another embodiment, the nucleic acid construct further comprises a tracrRNA sequence. Again, an appropriate tracrRNA sequence would be known to the skilled person as this sequence is defined by the choice of Cas protein. Nonetheless, in one embodiment said sequence comprises or consists of a sequence as defined in SEQ ID NO: 47 (used successfully in wheat in Shan et al. 2014) or a variant thereof.

In a further embodiment, the nucleic acid construct comprises at least one nucleic acid sequence that encodes a sgRNA (or gRNA). Again, as already discussed, sgRNA typically comprises a crRNA sequence or protospacer sequence and a tracrRNA sequence and preferably a sequence for a linker loop. In a preferred embodiment, the nucleic acid construct comprises at least one nucleic acid sequence that encodes a sgRNA sequence as defined in any of SEQ ID NO: 52 to 55 or variant thereof. More preferably the nucleic acid sequence that encodes a sgRNA comprises or consists of a sequence selected from SEQ ID NO: 48 to 51 or a variant thereof.

In a further embodiment, the nucleic acid construct may further comprise at least one nucleic acid sequence encoding an endoribonuclease cleavage site. Preferably the endoribonuclease is Csy4 (also known as Cas6f). Where the nucleic acid construct comprises multiple sgRNA nucleic acid sequences the construct may comprise the same number of endoribonuclease cleavage sites. In another embodiment, the cleavage site is 5′ of the sgRNA nucleic acid sequence. Accordingly, each sgRNA nucleic acid sequence is flanked by a endoribonuclease cleavage site.

The term ‘variant’ refers to a nucleotide sequence where the nucleotides are substantially identical to one of the above sequences. The variant may be achieved by modifications such as insertion, substitution or deletion of one or more nucleotides. In a preferred embodiment, the variant has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to any one of the above described sequences. In one embodiment, sequence identity is at least 90%. In another embodiment, sequence identity is 100%. Sequence identity can be determined by any one known sequence alignment program in the art.

The invention also relates to a nucleic acid construct comprising a nucleic acid sequence operably linked to a suitable plant promoter. A suitable plant promoter may be a constitutive or strong promoter or may be a tissues-specific promoter. In one embodiment, suitable plant promoters are selected from, but not limited to, cestrum yellow leaf curling virus (CmYLCV) promoter or switchgrass ubiquitin 1 promoter (PvUbi1) wheat U6 RNA polymerase III (TaU6) CaMV35S, wheat U6 or maize ubiquitin (e.g. Ubi1) promoters. Alternatively, expression can be specifically directed to particular tissues of wheat seeds through gene expression-regulating sequences. In one embodiment, the promoter is selected from the U6 promoter (for example as defined in SEQ ID NO: 58) and the ubiquitin 1 promoter (for example as defined in SEQ ID NO: 59). In a preferred embodiment the sequences are codon-optimised for the plant in question.

The nucleic acid construct of the present invention may also further comprise a nucleic acid sequence that encodes a CRISPR enzyme. By “CRISPR enzyme” is meant an RNA-guided DNA endonuclease that can associate with the CRISPR system. Specifically, such an enzyme binds to the tracrRNA sequence. In one embodiment, the CRIPSR enzyme is a Cas protein (“CRISPR associated protein), preferably Cas 9 or Cpf1, more preferably Cas9. In a specific embodiment Cas9 is codon-optimised Cas9 (optimised for the plant in which it is expressed). In one example, Cas9 has the sequence described in SEQ ID NO: 56 or a functional variant or homolog thereof. In another embodiment, the CRISPR enzyme is a protein from the family of Class 2 candidate×proteins, such as C2c1, C2C2 and/or C2c3. In one embodiment, the Cas protein is from Streptococcus pyogenes. In an alternative embodiment, the Cas protein may be from any one of Staphylococcus aureus, Neisseria meningitides, Streptococcus thermophiles or Treponema denticola.

The term “functional variant” as used herein with reference to Cas9 refers to a variant Cas9 gene sequence or part of the gene sequence which retains the biological function of the full non-variant sequence, for example, acts as a DNA endonuclease, or recognition and/or binding to DNA. A functional variant also comprises a variant of the gene of interest which has sequence alterations that do not affect function, for example non-conserved residues. Also encompassed is a variant that is substantially identical, i.e. has only some sequence variations, for example in non-conserved residues, compared to the wild type sequences as shown herein and is biologically active. In one embodiment, a functional variant of SEQ ID NO. 56 has at least 90%, 91%, 92%, 93%, 94%, 95%, 96%, 97%, 98%, or 99% overall sequence identity to the nucleic acid represented by SEQ ID NO: 56. In a further embodiment, the Cas9 protein has been modified to improve activity.

Suitable homologs or orthologs can be identified by sequence comparisons and identifications of conserved domains. The function of the homolog or ortholog can be identified as described herein and a skilled person would thus be able to confirm the function when expressed in a plant.

In a further embodiment, the Cas9 protein has been modified to improve activity. For example, in one embodiment, the Cas9 protein may comprise the D10A amino acid substitution, this nickase cleaves only the DNA strand that is complementary to and recognized by the gRNA. In an alternative embodiment, the Cas9 protein may alternatively or additionally comprise the H840A amino acid substitution, this nickase cleaves only the DNA strand that does not interact with the sRNA. In this embodiment, Cas9 may be used with a pair (i.e. two) sgRNA molecules (or a construct expressing such a pair) and as a result can cleave the target region on the opposite DNA strand, with the possibility of improving specificity by 100-1500 fold. In a further embodiment, the Cas9 protein may comprise a D1135E substitution. The Cas 9 protein may also be the VQR variant. Alternatively, the Cas protein may be comprise a mutation in both nuclease domains, HNH and RuvC0 like and therefore is catalytically inactive. Rather than cleaving the target strand, this catalytically inactive Cas protein can be used to prevent the transcription elongation process, leading to a loss of function of incompletely translated proteins when co-expressed with a sgRNA molecule. An example of a catalytically inactive protein is dead Cas9 (dCas9) caused by a point mutation in RuvC and/or the HNH nuclease domains (Komor et al., 2016 and Nishida et al., 2016).

In a further embodiment, a Cas protein, such as Cas9 may be further fused with a repression effector, such as a histone-modifying/DNA methylation enzyme or a Cytidine deaminase (Komor et al. 2016) to effect site-directed mutagenesis. In the latter, the cytidine deaminase enzyme does not induce dsDNA breaks, but mediates the conversion of cytidine to uridine, thereby effecting a C to T (or G to A) substitution.

In a further embodiment, the nucleic acid construct comprises an endoribonuclease. Preferably the endoribonuclease is Csy4 (also known as Cas6f) and more preferably a codon optimised csy4, for example as defined in SEQ ID NO: 57. In one embodiment, where the nucleic acid construct comprises a cas protein, the nucleic acid construct may comprise sequences for the expression of an endoribonuclease, such as Csy4 expressed as a 5′ terminal P2A fusion (used as a self-cleaving peptide) to a cas protein, such as Cas9.

In one embodiment, the cas protein, the endoribonuclease and/or the endoribonuclease-cas fusion sequence may be operably linked to a suitable plant promoter. Suitable plant promoters are already described above, but in one embodiment, may be the Zea mays Ubiquitin 1 promoter or U6 promoter.

Suitable methods for producing the CRISPR nucleic acids and vectors system are known, and for example are published in Molecular Plant (Ma et al., 2015, Molecular Plant, DOI:10.1016/j.molp.2015.04.007), which is incorporated herein by reference.

In an alternative aspect of the invention, the nucleic acid construct comprises at least one nucleic acid sequence that encodes a TAL effector, wherein said effector targets a MRC gene and/or promoter sequence, preferably selected from SEQ ID NO 40 to 43. Methods for designing a TAL effector would be well known to the skilled person, given the target sequence. Examples of suitable methods are given in Sanjana et al., and Cermak T et al, both incorporated herein by reference. Preferably, said nucleic acid construct comprises two nucleic acid sequences encoding a TAL effector, to produce a TALEN pair. In a further embodiment, the nucleic acid construct further comprises a sequence-specific nuclease (SSN). Preferably such SSN is a endonuclease such as FokI. In a further embodiment, the TALENs are assembled by the Golden Gate cloning method in a single plasmid or nucleic acid construct.

In another aspect of the invention, there is provided a sgRNA molecule, wherein the sgRNA molecule comprises a crRNA sequence and a tracrRNA sequence and wherein the crRNA sequence can bind to at least one sequence selected from SEQ ID NOs 39 to 42 or a variant thereof. In one embodiment, the nucleic sequence of the sgRNA molecule is defined in any of SEQ ID NO: 48 to 51 or variant thereof. In other words, the RNA sequence of the sgRNA is encoded by a nucleic acid sequence selected from SEQ ID NO: 48 to 51. In one example only, the RNA sequence of one sgRNA of the invention is defined in SEQ ID NO: 52 to 55 or a variant thereof. A “variant” is as defined herein. In one embodiment, the sgRNA molecule may comprise at least one chemical modification, for example that enhances its stability and/or binding affinity to the target sequence or the crRNA sequence to the tracrRNA sequence. Such modifications would be well known to the skilled person, and include for example, but not limited to, the modifications described in Rahdar et al., 2015, incorporated herein by reference. In this example the crRNA may comprise a phosphorothioate backbone modification, such as 2′-fluoro (2′-F), 2′-O-methyl (2′-O-Me) and S-constrained ethyl (cET) substitutions.

In another aspect of the invention, there is provided an isolated nucleic acid sequence that encodes for a protospacer element (as defined in any of SEQ ID NOs 43 to 46), or a sgRNA (as described in any of SEQ ID NO: 48 to 51). There is also provided an isolated sgRNA molecule as defined in any of SEQ ID NO: 52 to 55.

In another aspect of the invention, there is provided a plant or part thereof or at least one isolated plant cell transfected with at least one nucleic acid construct as described herein. Cas9 and sgRNA may be combined or in separate expression vectors (or nucleic acid constructs, such terms are used interchangeably). In other words, in one embodiment, an isolated plant cell is transfected with a single nucleic acid construct comprising both sgRNA and Cas9 as described in detail above. In an alternative embodiment, an isolated plant cell is transfected with two nucleic acid constructs, a first nucleic acid construct comprising at least one sgRNA as defined above and a second nucleic acid construct comprising Cas9 or a functional variant or homolog thereof. The second nucleic acid construct may be transfected below, after or concurrently with the first nucleic acid construct. The advantage of a separate, second construct comprising a cas protein is that the nucleic acid construct encoding at least one sgRNA can be paired with any type of cas protein, as described herein, and therefore are not limited to a single cas function (as would be the case when both cas and sgRNA are encoded on the same nucleic acid construct).

In one embodiment, the nucleic acid construct comprising a cas protein is transfected first and is stably incorporated into the genome, before the second transfection with a nucleic acid construct comprising at least one sgRNA nucleic acid. In an alternative embodiment, a plant or part thereof or at least one isolated plant cell is transfected with mRNA encoding a cas protein and co-transfected with at least one nucleic acid construct as defined herein. Alternatively, as described in Example 2, the nucleic acid constructs can be transiently expressed in the target plant cell.

Cas9 expression vectors for use in the present invention can be constructed as described in the art. In one example, the expression vector comprises a nucleic acid sequence as defined in SEQ ID NO: 56 or a functional variant or homolog thereof, wherein said nucleic acid sequence is operably linked to a suitable promoter. Examples of suitable promoters include the Actin, CaMV35S, wheat U6 or maize ubiquitin (e.g. Ubi1) promoter, as described above.

In an alternative aspect of the present invention, there is provided an isolated plant cell (transiently or stably) transfected with at least one nucleic acid construct or sgRNA molecule as described herein.

In a further aspect of the invention, there is provided a genetically modified or edited plant comprising the transfected cell described herein. In one embodiment, the nucleic acid construct or constructs may be integrated in a stable form. In an alternative embodiment, the nucleic acid construct or constructs are not integrated (i.e. are transiently expressed). Accordingly, in a preferred embodiment, the genetically modified plant is free of any sgRNA and/or Cas protein nucleic acid. In other words, the plant is transgene free.

The term “introduction”, “transfection” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art.

The transfer of foreign genes into the genome of a plant is called transformation. Transformation of plants is now a routine technique in many species. Any of several transformation methods known to the skilled person may be used to introduce the nucleic acid construct or sgRNA molecule of interest into a suitable ancestor cell. The methods described for the transformation and regeneration of plants from plant tissues or plant cells may be utilized for transient or for stable transformation.

Transformation methods include the use of liposomes, electroporation, chemicals that increase free DNA uptake, injection of the DNA directly into the plant (microinjection), gene guns (or biolistic particle delivery systems (biolistics)) as described in the examples, lipofection, transformation using viruses or pollen and microprojection. Methods may be selected from the calcium/polyethylene glycol method for protoplasts, ultrasound-mediated gene transfection, optical or laser transfection, transfection using silicon carbide fibers, electroporation of protoplasts, microinjection into plant material, DNA or RNA-coated particle bombardment, infection with (non-integrative) viruses and the like. Transgenic plants, can also be produced via Agrobacterium tumefaciens mediated transformation, including but not limited to using the floral dip/Agrobacterium vacuum infiltration method as described in Clough & Bent (1998) and incorporated herein by reference.

Accordingly, in one embodiment, at least one nucleic acid construct or sgRNA molecule as described herein can be introduced to at least one plant cell using any of the above described methods. In an alternative embodiment, any of the nucleic acid constructs described herein may be first transcribed to form a preassembled Cas9-sgRNA ribonucleoprotein and then delivered to at least one plant cell using any of the above described methods, such as lipofection, electroporation or microinjection.

Optionally, to select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. As described in the examples, a suitable marker can be bar-phosphinothricin or PPT. Alternatively, the transformed plants are screened for the presence of a selectable marker, such as, but not limited to, GFP, GUS (β-glucuronidase). Other examples would be readily known to the skilled person. Alternatively, no selection is performed, and the seeds obtained in the above-described manner are planted and grown and MRC expression or protein levels measured at an appropriate time using standard techniques in the art. This alternative, which avoids the introduction of transgenes, is preferable to produce transgene-free plants.

Following DNA transfer and regeneration, putatively transformed plants may also be evaluated, for instance using PCR to detect the presence of the gene of interest, copy number and/or genomic organisation. Alternatively or additionally, integration and expression levels of the newly introduced DNA may be monitored using Southern, Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques.

In a further related aspect of the invention, there is also provided, a method of obtaining a genetically modified plant as described herein, the method comprising
a. selecting a part of the plant;
b. transfecting at least one cell of the part of the plant of paragraph (a) with at least one nucleic acid construct as described herein or at least one sgRNA molecule as described herein, using the transfection or transformation techniques described above;
c. regenerating at least one plant derived from the transfected cell or cells;
d. selecting one or more plants obtained according to paragraph (c) that show altered expression or activity of MRC.

In a further embodiment, the method also comprises the step of screening the genetically modified plant for SSN (preferably CRISPR)-induced mutations in the MRC gene or promoter sequence. In one embodiment, the method comprises obtaining a DNA sample from a transformed plant and carrying out DNA amplification to detect a mutation in at least one MRC gene or promoter sequence.

In a further embodiment, the methods comprise generating stable T2 plants preferably homozygous for the mutation (that is a mutation in in at least one MRC gene or promoter sequence).

Plants that have a mutation in at least one MRC gene or promoter sequence can also be crossed with another plant also containing at least one mutation in at least one MRC gene or promoter sequence to obtain plants with additional mutations in the MRC gene or promoter sequence. The combinations will be apparent to the skilled person. Accordingly, this method can be used to generate a T2 plants with mutations on all or an increased number of homoelogs, when compared to the number of homoeolog mutations in a single T1 plant transformed as described above.

A plant obtained or obtainable by the methods described above is also within the scope of the invention.

A genetically altered plant of the present invention may also be obtained by transference of any of the sequences of the invention by crossing, e.g., using pollen of the genetically altered plant described herein to pollinate a wild-type or control plant, or pollinating the gynoecia of plants described herein with other pollen that does not contain a mutation in at least one of the MRC gene or promoter sequence. The methods for obtaining the plant of the invention are not exclusively limited to those described in this paragraph; for example, genetic transformation of germ cells from the ear of wheat could be carried out as mentioned, but without having to regenerate a plant afterward.

In a further final aspect of the invention, there is provided a method of screening a population of plants and identifying and/or selecting a plant that will have altered expression and/or activity of MRC and therefore an alteration in granule size distribution in a plant, as described herein, compared to a control or wild-type plant, the method comprising detecting at least one polymorphism or mutation in the MRC gene and/or promoter, wherein said mutation or polymorphism leads to an alteration in the level of expression and/or activity of the MRC protein compared to the level in a plant not carrying said mutation or polymorphism (e.g. a control or wild-type plant). Said mutation or polymorphism may comprise at least one insertion and/or at least one deletion and/or substitution.

Suitable tests for assessing the presence of a polymorphism would be well known to the skilled person, and include but are not limited to, Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs-which are also referred to as Microsatellites), and Single Nucleotide Polymorphisms (SNPs). In one embodiment, Kompetitive Allele Specific PCR (KASP) genotyping is used.

The method may also comprise the step of assessing whether the polymorphism has an effect on starch granule size distribution as described herein. Methods to screen for an effect on granule size distribution would be well known to the skilled person, but could be carried out using a coulter counter, a microscope, a flow cytometer or any other particle sizing instrument.

In one embodiment, the method comprises
a) obtaining a nucleic acid sample from a plant and
b) carrying out nucleic acid amplification of one or more MRC gene and/or promoter alleles using one or more primer pairs.

In a further embodiment, the method may further comprise introgressing the chromosomal region comprising at least one of said low-MRC-expressing/activity polymorphisms into a second plant or plant germplasm to produce an introgressed plant or plant germplasm. Preferably the expression or activity of MRC in said second plant will be altered (compared to a control or wild-type plant), and more preferably said second plant will display an alteration in at least one of starch granule size, number and distribution, as described above.

In a further aspect of the invention there is provided a method of altering starch granule size distribution, as described above in a plant, the method comprising
a. screening a population of plants for at least one plant with at least one of the above described polymorphisms or mutations; and
b. further altering (i.e. reducing/abolishing or increasing) the expression of at least one MRC nucleic acid and/or altering (i.e. reducing/abolishing or increasing) the activity of a MRC polypeptide in said plant by introducing at least one mutation into the nucleic acid sequence encoding MRC or at least one mutation into the promoter of MRC as described herein or using RNA interference as described herein.

By “further altering” is meant reducing or increasing the level of MRC expression to a level lower or higher than that in the plant with the at least one of the above-described MRC polymorphisms. The terms “reducing” or “increasing” means a decrease or increase in the levels of MRC expression and/or activity by up to 10%, 20%, 30%, 40%, 50%, 60%, 70%, 80% or 90% when compared to the level in a control plant.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting examples.

### Example I

### MRC Orthologs are Encoded on Chromosomes 6A and 6D

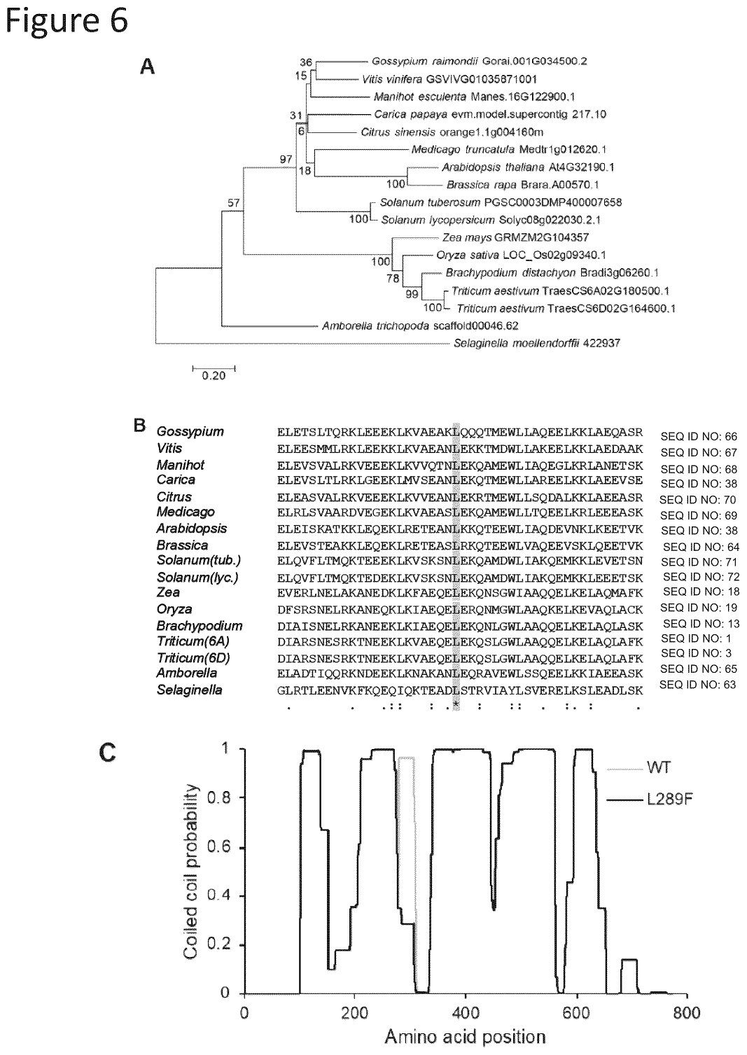

As a first step to determining the role of the starch granule initiation protein, MRC, in wheat endosperm, we scanned the wheat genome for genes encoding MRC orthologs. We ran a BLASTp search using the amino acid sequence of Arabidopsis MRC (AtMRC, At4g32190) against the protein database from the RefSeq v1.0 wheat genome on Ensembl plants. The two top protein hits, TraesCS6D02G164600.1 (encoded on chromosome 6D) and TraesCS6A02G180500.1 (encoded on chromosome 6A) both had 33% identity to AtMRC and had low E values (7.6E-39 and 1.0E-38, respectively). The two wheat proteins shared 95% identity, suggesting that they could be homeologs. However, there was no protein hit corresponding to a homeolog on chromosome 6B. To determine whether the 6A- and 6D-encoded proteins were true orthologs of AtMRC, we repeated the phylogenetic analyses of MRC homologs from our previous study (Seung et al., 2018) and included the wheat protein sequences. The 6A and 6D proteins grouped closely together on the tree, distinctly within the grass clade containing the rice and maize sequences (FIG. 6). This confirms that the two proteins are the wheat orthologs of MRC, and will hereafter be referred as TaMRC-6A (TraesCS6A02G180500) and TaMRC-6D (TraesCS6D02G164600).

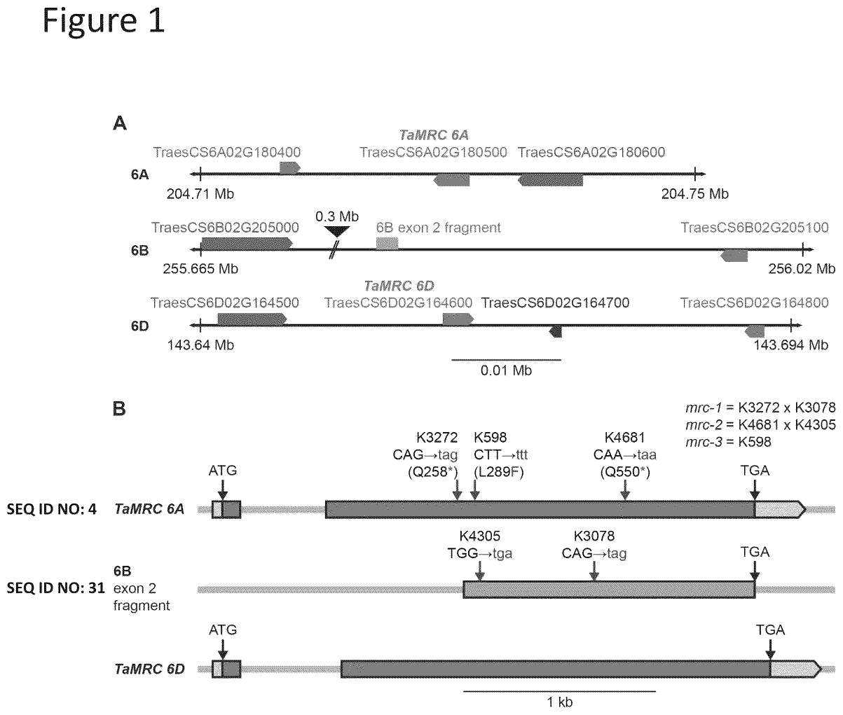

To examine the wheat gene models and investigate why no homeolog was detected on chromosome 6B, we looked at the genomic regions around the TaMRC-6A and TaMRC-6D loci. The loci were in syntenous positions on the A and D genomes, confirming that they are homeologs (FIG. 1A). Close examination of the gene model showed that the loci had a two-exon structure (FIG. 1B), like the Arabidopsis gene (At4g32190.1; Seung et al., 2018). In the syntenous region on chromosome 6B, there was a stretch of sequence that had homology to exon 2, but we failed to find regions with homology to exon 1 upstream of this sequence. Thus, we could not find a full TaMRC gene model in the syntenic position on chromosome 6B, or anywhere else on the B genome. Interestingly, the distance between the exon 2 fragment on chromosome 6B and its upstream neighbouring gene (a respiratory burst oxidase homolog) was much larger than the distance between TaMRC and the homeologs of the same neighbouring gene on 6A and 6D (FIG. 1A). This led us to hypothesise that the 6B copy had been interrupted by a large insertion and had become a pseudogene during the course of wheat evolution.

We then looked at expression data of TaMRC orthologs using the wheat expression browser (Borrill et al., 2016). Transcripts of the 6A and 6D homeologs were detected in both leaves and grains, suggesting that MRC plays a role in these tissues. Transcripts for both homeologs were also detected in the developing endosperm tissue, but only during the early stages of grain development before 12 dpa.

To study the function of MRC in starch synthesis in the wheat endosperm, we obtained mutants in tetraploid wheat (Triticum turgidum) defective in MRC. We used the wheat in silico TILLING mutant resource, which contains an EMS-mutagenised population of the durum wheat cultivar, Kronos, and exome-capture sequencing data for identification of lines with mutations of interest (http://www.wheat-tilling.com; Krasileva et al., 2017). We ordered three mutants that were likely to cause a loss of function in TaMRC-6A (FIG. 1B). The K3272 and K4681 lines contained premature stop codons after the 257^{th} and 550^{th} amino acids respectively. In addition, we ordered a third line that contained a missense Leu289Phe mutation, which was predicted to be deleterious to protein function by SIFT scoring (Ng and Henikoff, 2006). The Leu²⁸⁹ residue is highly conserved in all MRC orthologs, and its mutation to a Phe residue is predicted to disrupt coiled coil formation in the region of the residue (FIG. 6).

Since tetraploid wheat lacks a D genome, and the 6B homeolog of MRC has likely become a pseudogene, we predicted that TaMRC-6A would be the only functional MRC homeolog in T. turgidum. However, to rule out the possibility that the fragment of exon 2 on chromosome 6B has an effect on MRC function, we also obtained the K4305 and K3078 lines, which contain two different premature stop codon mutations in the putative reading frame of the exon. We generated the mrc-1 lines by crossing K3272 and K3078, and isolated lines homozygous for either the 6A or 6B mutation, or both. The mrc-2 lines were generated in the same way, but the K4681 and K4305 lines were crossed. The mrc-3 line contained the K598 missense mutation, and no crossing was conducted.

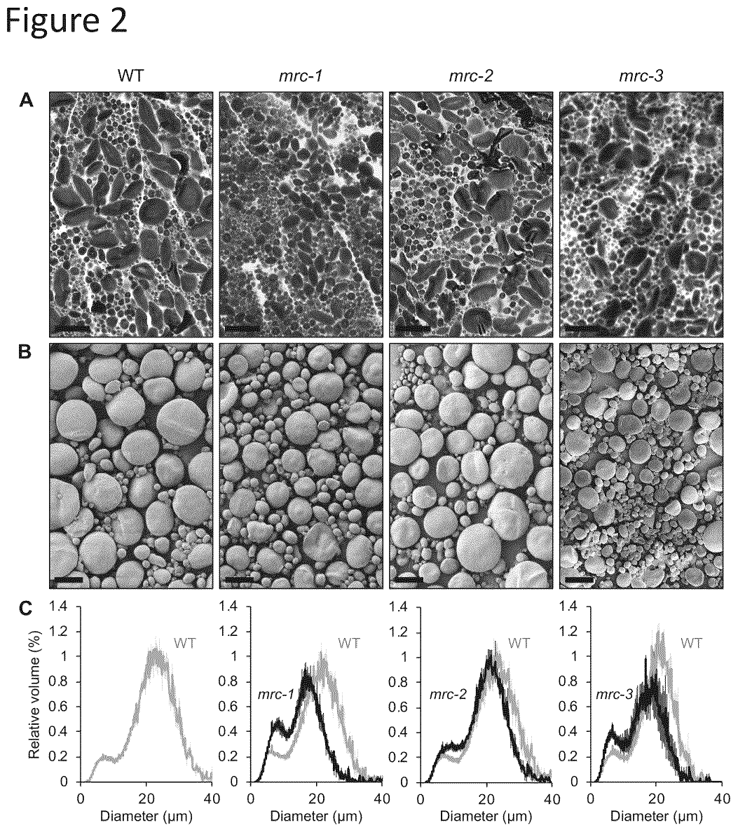

MRC is required for normal starch granule size distributions in the endosperm Since MRC is involved in granule initiation in Arabidopsis, we hypothesised that our wheat mutants would have fewer, but larger starch granules in the endosperm. We therefore first examined starch granule size in iodine-stained thin sections of mature grains of the wheat mrc mutants using light microscopy. The endosperms of the wild type and the mutant grains contained both large A-type granules and smaller B-type granules. However, the mrc-1 and mrc-3 mutants had a noticeable change in granule size, both appearing to contain smaller A-type granules and more B-type granules than the wild type (FIG. 2A). To gain a more detailed view of starch granule size and morphology, we purified granules from mature grains of the wild type and mutants, and examined them with scanning electron microscopy (SEM). Similar to the observations made in the sections of FIG. 2A, the mrc-1 and mrc-3 mutants had smaller A-type granules and more numerous B-type granules than the wild type. Aside from the altered granule size, none of the mutants showed any defect in granule shape: the A-type granules in the wild type and mutants had their characteristic flattened morphology, while the B-type granules had near-spherical morphology that sometimes contained flattened edges (FIG. 2B).

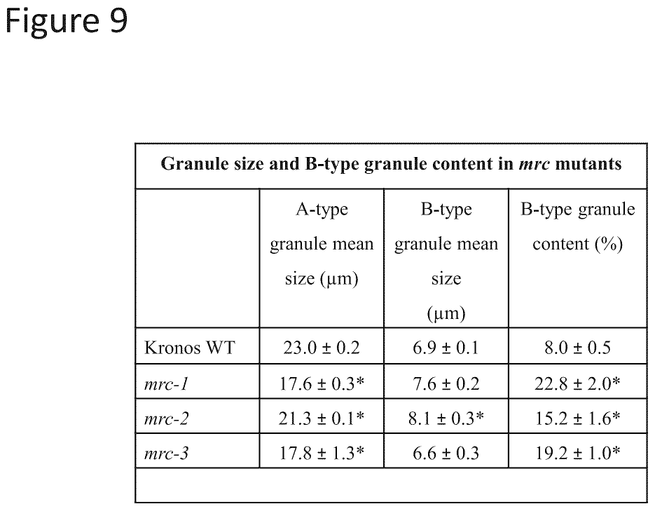

We used a coulter counter to quantify starch granule size in the mutants. Granule size distributions were plotted by measuring the size of at least 100,000 purified starch granules from the wild type and mutants. We observed clear bimodal distributions for all genotypes, with a peak corresponding to A-type granules (>10 μm), and a peak corresponding to B-type granules (<10 μm). However, the area of the B-type granule peak was larger in all mrc mutants than in the wild-type, indicating that starch from the mutants contain a higher proportion of B-type granules (by volume) than wild-type starch (FIG. 2C). Also, the A-type granule peak in the mutants was shifted towards the smaller size range. These alterations in granule size distribution were more severe in mrc-1 and mrc-3 than in mrc-2. We fitted a bimodal mixed gaussian distribution to the data to estimate the total volume of B-type granules, as well as the mean size of the A-type and B-type granules. In mrc-1 and mrc-3, the B-type fraction constituted 19-23% of total starch volume, which was higher than in the mrc-2 mutant (15%) and the wild type (8%) (FIG. 9). However, the mean size of the B-type granules was not significantly altered in the mutants, with the exception of mrc-2, where they were slightly larger. This suggests that the increased proportion of B-type granules in the mrc mutants is primary due to a change in A- to B-type granule ratio rather than larger individual B-type granules. However, the increase in the proportion of B-type granules in the mutants was accompanied by a significant decrease in A-type granule size.

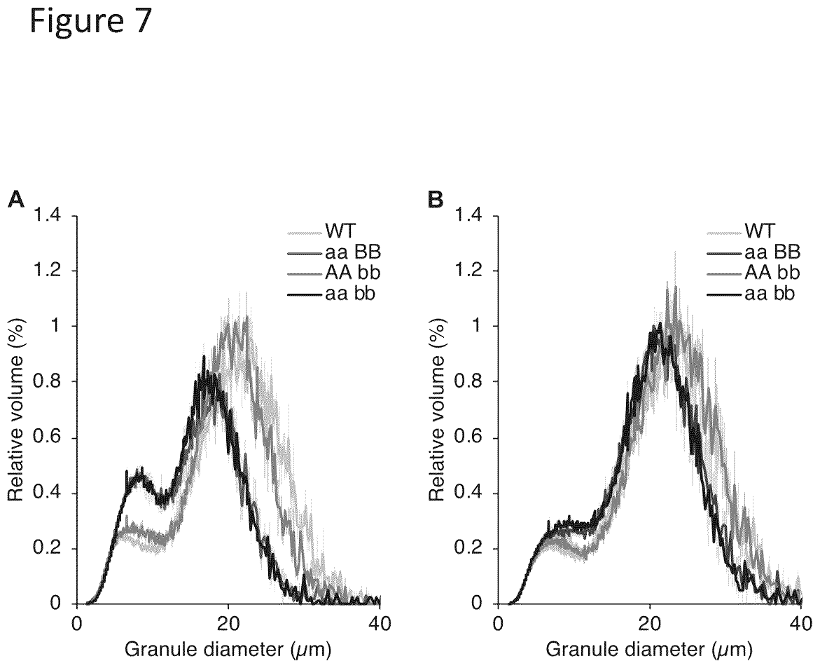

To confirm that the TaMRC-6A is the only functional homeolog of MRC in tetraploid wheat, we quantified starch granule size distribution in the full set of homozygous genotypes resulting from the crosses that yielded the mrc-1 and mrc-2 mutants [indicated as aa BB (6A mutant), AA bb (6B mutant) and aa bb (6A and 6B double mutant)]. The size distribution of starch granules was identical between wild type and AA bb, and were similarly altered in aa BB and aa bb genotypes. This provides experimental evidence that the fragment of exon 2 on chromosome 6B does not contribute to granule size distribution and is likely to be a pseudogene (FIG. 7).

These data suggest that TaMRC is required for the normal size distribution of starch granules in wheat endosperm. The difference in the severity of the phenotype between the different mrc mutants could be explained by the position of the premature stop codons in the MRC coding sequence. In mrc-1, the premature stop codon occurs earlier in the coding sequence than in mrc-2. It is possible that the truncated protein in mrc-2 is partially functional. The size distribution of mrc-3 was very similar to that of mrc-1, suggesting that the Leu289Phe mutation severely inhibits protein function.

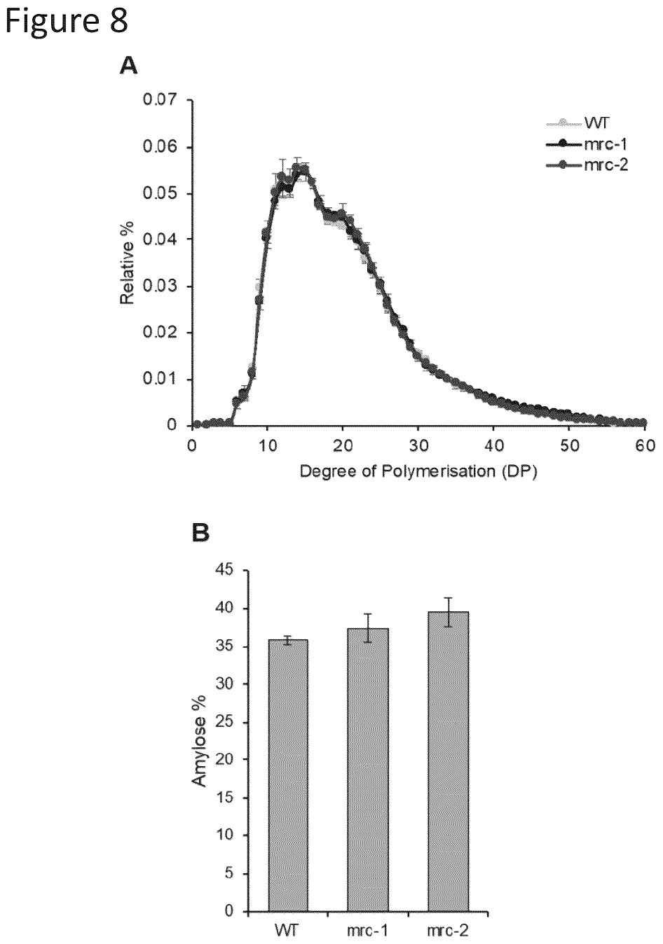

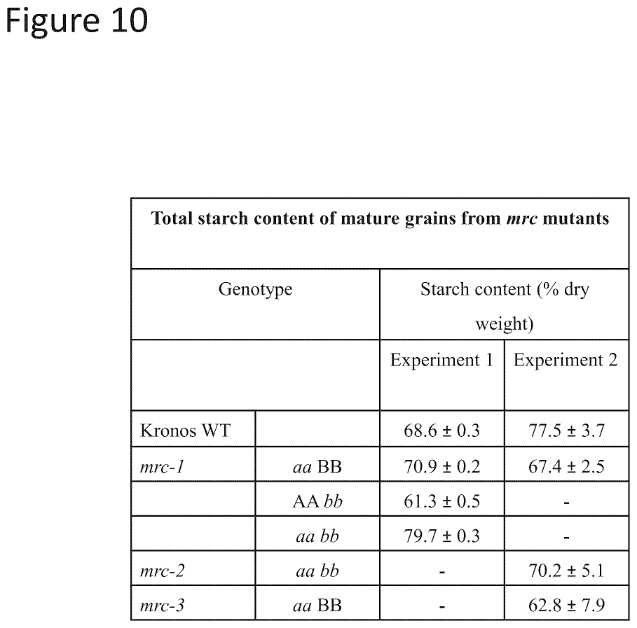

To test whether these changes in granule size distribution were accompanied by altered total starch content, we measured total starch in the mature grains of the mrc mutants, but we did not observe any significant differences (FIG. 10). We also tested whether starch polymer structure or composition was altered in the mutant. However, even the mutant with the strongest alteration in granule size distribution had normal amylopectin structure and amylose content (FIG. 8). The mrc-1 mutant produced the same grain yield per plant as the wild type and did not have any noticeable growth defects.

### Loss of MRC Results in the Early Initiation of B-Type Granules

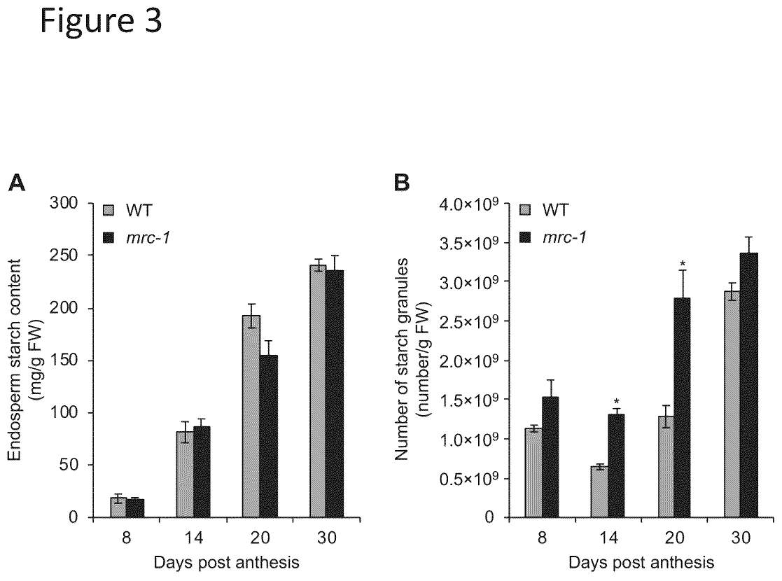

In wheat endosperm, loss of MRC alters the ratio of A-type and B-type granules, in favour of B-type granules. We reasoned that this could result from two possible scenarios: 1) loss of MRC reduces the number of A-type granules, resulting in a relative increase in B-type granules, or 2) loss of MRC results in more B-type granules and does not affect the number of A-type granules. To distinguish between these two possibilities, we investigated granule initiation in the developing endosperm of the mrc-1 mutant in more detail. We dissected the endosperm of developing grains harvested 8, 14, 20 and 30 days post anthesis (dpa), and measured both the total starch content and numbers of starch granules. The total starch content of the endosperm increased between each time point, and there was no significant difference between the mutant and the wild type at any time point (FIG. 3A). We then quantified the total number of starch granules in the endosperm using the coulter counter. At the 8 dpa timepoint, the mutant and the wild type contained a similar number of starch granules. Interestingly, for the two subsequent time points (14 dpa and 20 dpa), the mutant endosperms contained almost double the number of starch granules of the wild type, despite similar starch contents (FIG. 3B). The largest increase in granule number during grain filling was observed between the 20 and 30 dpa timepoints in the wild type, but was between the 14 and 20 dpa timepoints in the mutant. At the 30 dpa timepoint, the difference in granule number between the mutant and the wild type became less apparent, with no significant differences observed. However, quantification of starch granule number in mature grains showed that the mrc-1 mutant [(201±12)×10⁶ granules per grain] had significantly more granules than wild type grains [(157±7)×10⁶ granules per grain](p<0.05 under a two-tailed t-test). We also noted that in both the wild type and mutant, the number of starch granules decreased between 8 and 15 dpa timepoints. The reason for this is unknown, but it has also been observed in previous studies (Howard et al., 2011).

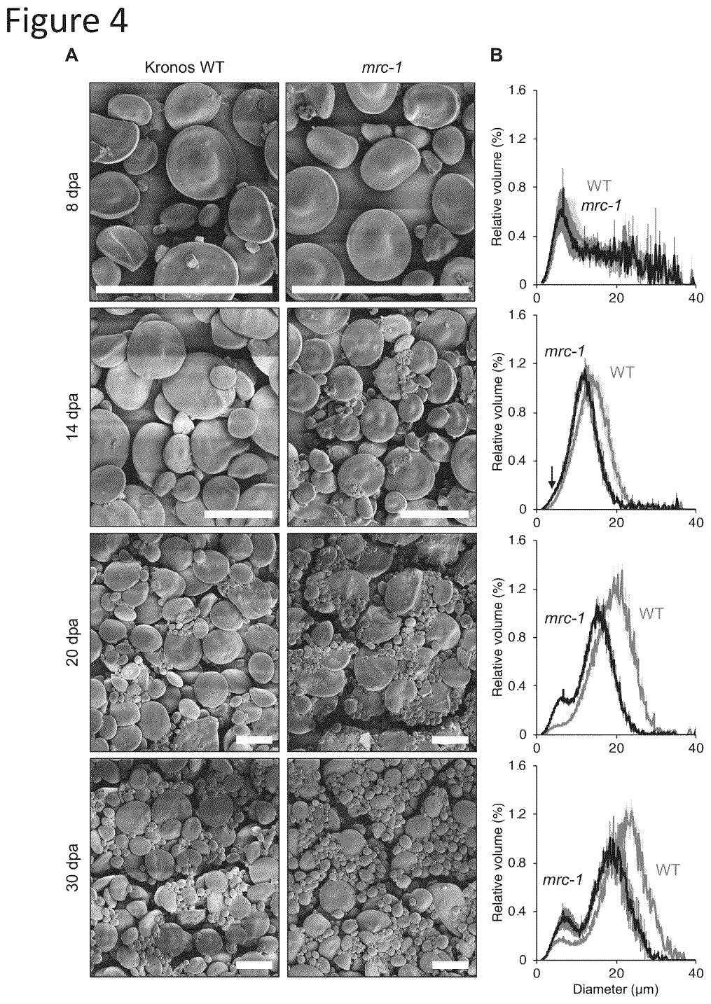

To observe changes in A- to B-type granule ratio throughout grain development, we used both scanning electron microscopy and the coulter counter to examine the morphology and size of endosperm starch granules. In the wild type, only A-type granules with their characteristic flattened morphology were observed at the 8 and 14 dpa timepoints, but they grew substantially in size between the two timepoints (FIG. 4). This was consistent with the size distributions from the coulter counter, which showed unimodal distributions for wild-type at both time points. Small, round B-type granules only became prominent at the 20 dpa timepoint in the wild type, but were already present at the 14 dpa timepoint in the mrc-1 mutant. This could be seen as a distinct shoulder that appeared in the granule size distribution of mrc-1 starch at the 14 dpa timepoint, and bimodal distributions at subsequent time points. The A-type granules in the mutant were the same size as those of the wild type at the 8 dpa time point, but gradually became smaller as grain development progressed, particularly as B-type granules were initiated. Taken together, these data suggest that the elevated number of granules between 14-20 dpa in mrc-1 endosperm (observed in FIG. 3B) is due to the premature initiation of B-type granules in the mutant. Presumably, this also restricts the growth of the A-type granules as they compete with the B-type granules for the substrates of starch synthesis.

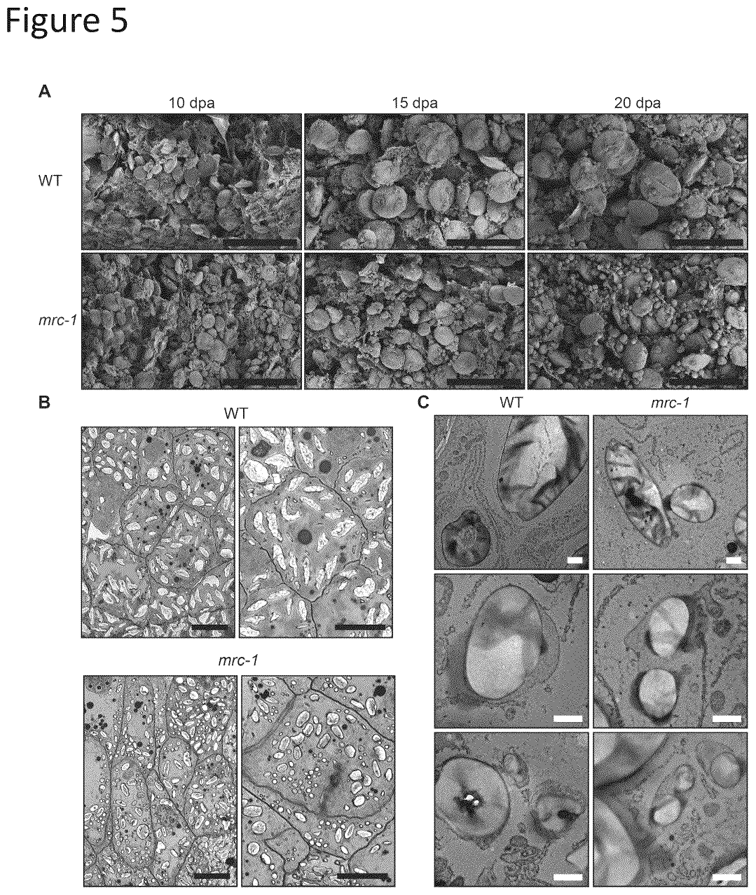

Normally within amyloplasts, several B-type granules initiate in close proximity—appearing as ‘clusters’ in between the A-type granules—and at least some B-type granules form in amyloplast stromules (Parker, 1985; Langeveld et al., 2000). Given the unusual timing of B-type granule initiation in mrc-1, we explored whether the loss of MRC also affected the location of B-type granule initiation. First, we harvested grains during their development, subjected them to critical point drying, and imaged sections through the endosperm tissue using SEM. Consistent with the findings from the purified starch granules, B-type granules were prominent already at 15 dpa in the mutant, whereas they only became prominent after 20 dpa in the wild type (FIG. 5A). The B-type granules occurred in clusters in the mutant that resembled those of the wild type. Interestingly, even at the 10 dpa timepoint, clusters of tiny B-type granules were visible in the mutant. In a second approach, we produced sections from developing grain tissue (15 dpa) embedded in resin blocks for imaging by light and electron microscopy. For light microscopy, sections were stained with toluidine blue (a negative stain for starch). At 15 dpa, the most starch granules in the wild type endosperm were flattened A-type granules, and there were very few B-type granules visible (FIG. 5B). However, in the endosperm of the mrc-1 mutant, many clustered B-type granules were present at this time point. Thus, we used transmission electron microscopy (TEM) to investigate whether the multiple B-type granules in mrc-1 occurred within single amyloplasts, particularly in stromules. Indeed, multiple B-type granules were enclosed within a single set of amyloplast membranes, and the elongated morphology of these amyloplast regions strongly suggested that they are stromules (FIG. 5C). Stromules are difficult to observe in two-dimensional electron microscopy sections, and it is difficult to determine the exact percentage of B-type granules in stromules relative to those that are not. Overall, aside from their earlier occurrence in the mutant, we did not notice anything unusual about the location of B-type granules in the mrc-1 mutant.

To conclude, B-type granule initiation during grain development is under both spatial and temporal control. MRC appears to be required for this temporal control, as its absence stimulates the early formation of B-type granules. However, no defects in the spatial control of B-type granule formation were observed in mrc-1 mutants. We therefore propose that MRC acts as a repressor of B-type granule formation in the developing wheat endosperm.

### A Novel Role for MRC in Endosperm Starch Synthesis

Factors that regulate the unique spatio-temporal pattern of starch granule initiation in Triticeae endosperm are poorly understood. Here, we discovered a novel role for MRC in the temporal control of granule initiation in the wheat endosperm. Starch from multiple independent TILLING mutants of tetraploid wheat defective in TaMRC-6A had a higher percentage volume of B-type granules than the wild type (FIG. 2). These B-type granules initiated in the mutant up to 10 days earlier than in the wild type (FIGS. 3-5). Despite the unusual timing of initiation, the B-type granules in the mutant had normal round morphology, and as in the wild type, at least some were in amyloplast stromules. Further, the mutants had normal total starch content, amylopectin structure and amylose content. Thus in wheat endosperm, as in Arabidopsis leaves, MRC appears to be specifically involved in controlling the initiation step of starch biosynthesis. The premature initiation of B-type granules also appears to have no effect on total starch synthesis rates in the endosperm (FIG. 3; FIG. 10).

Interestingly, MRC is required for the temporal control of B-type granule initiation, but A-type granule initiation appears to proceed normally in its absence. We demonstrated that the increased proportion of B-type granule volume in starch of the mrc mutant does not arise from a suppression of A-type granule initiation. If this were to be the case, we would have expected fewer, larger A-type granules in the mutant. However, at the early stages of grain development (8 dpa), the size, shape and number of A-type granules were identical between the mutant and the wild type (FIGS. 3-4). It was only at the later stages of grain development following B-type granule initiation that the A-type granules became distinctively smaller in the mutant compared to the wild-type. It is likely that the A-type granules in the mutant compete with the early-initiated B-type granules for substrates of granule growth (i.e.: ADP-Glucose). The final number of starch granules per grain was about 25-30% greater in the mutant than the wild type, and granule numbers in the endosperm of both mutant and wild type increased steadily throughout grain development—indicating that granules are continuously initiated after the main wave of B-type granule initiated, albeit at a lower rate. Thus the early initiation of B-type granules in the mutant results in an increased final number of B-type granules. Interestingly, we did not pick up on consistent differences in B-type granule size between mutant and wild type. Taken together, the mrc mutant contains more B-type granules by percentage volume than the wild type because it has a higher number of B-type granules and the same number of smaller A-type granules that occupy less volume.

Overall, our findings that MRC specifically affects the timing of B-type granule initiation supports the hypothesis that the initiation of A- and B-type granules occur through distinct biochemical mechanisms. This hypothesis is also supported by mutants in various Triticeae with reduced B-type granule abundance (e.g.: PTST2 knockdown mutants, discussed below)(Howard et al., 2011; Chia et al., 2017, 2019), and natural variation in B-type granule content (Stoddard, 1999; Stoddard and Sarker, 2000).

### MRC can Suppress or Promote Granule Initiation Depending on Tissue

Our work reveals that MRC can both suppress or promote granule initiation depending on tissue. MRC appears to limit B-type granule initiation in the endosperm during early grain development, but promotes granule initiation in leaf chloroplasts. Consistent with this model, MRC is expressed primarily during early grain development, but not at later stages (FIG. 10). By contrast, MRC appears to have an overall promotive effect on granule initiation in Arabidopsis, since chloroplasts of mrc mutants have much fewer starch granules than wild-type chloroplasts (Seung et al., 2018; Vandromme et al., 2018). The role of MRC in promoting granule initiation in leaf chloroplasts is conserved in wheat, since chloroplasts of wheat mrc mutants had a reduced number of starch granules compared to the wild type (FIG. 6). Thus, in wheat, MRC appears to promote granule initiation in leaves, and repress B-type granule initiation in the endosperm.

We do not know how MRC can exert opposite effects in different tissues. Wheat mutants with reduced gene dosage of PTST2 (also called FLO6) have fewer B-type granules with no apparent impact on A-type granules (Chia et al., 2017, 2019). This is the opposite phenotype of mrc mutants, and could suggest that PTST2 and MRC have opposing roles in B-type granule initiation in wheat endosperm. However, full knockout mutants in PTST2 have distorted granule morphology (including A-type granules), resulting from the initiation of compound granule-like structures (Chia et al., 2019). This suggests that PTST2 acts to promote B-type granule initiation, and suppress A-type granule initiation; whereas MRC exclusively suppresses B-type granule formation during early grain development.

The mechanism by which MRC acts in granule initiation in general is not known. Since MRC is a long coiled-coil protein with no known enzymatic domains, it is possible that it can exert opposite functions by interacting with different interaction partners. The Arabidopsis MRC interacts with other granule initiation proteins: it co-purified with PTST2 in leaf extracts (Seung et al., 2018), and interacted directly with SS4 in yeast-two-hybrid experiments (Vandromme et al., 2018). Consistent with these interactions, leaves of Arabidopsis mutants defective in MRC, PTST2 or SS4 all had reduced numbers of starch granules relative to wild type (Roldán et al., 2007; Seung et al., 2017, 2018). We are currently working to determine whether MRC acts with the same interaction partners in the endosperm. However, the lack of similarity between MRC and PTST2 mutants of wheat suggests that MRC may either interact with different partner proteins in the endosperm, or that the dynamics of the interactions between the known partner proteins is different. Interestingly, the Arabidopsis MRC interacts directly with SS4 in yeast-two-hybrid experiments (Vandromme et al., 2018). However, the effect of a full SS4 knockout on granule numbers beyond Arabidopsis leaves in wheat is not yet known.

Alternatively, MRC may play a different role in the endosperm due to a difference in MRC localisation in the endosperm. In Arabidopsis chloroplasts, MRC localises to discrete puncta (Seung et al., 2018). We do not with know where MRC localises in wheat endosperm amyloplasts, particularly in relation to the stromules that contain B-type granules. Also, the fact that B-type granules initiated early in the mrc mutant suggests that there is no substrate-limitation that restricts the formation B-type granule formation earlier in grain development, for example maltooligosaccharides that may act as primers for granule initiation. The formation of stromules does not appear to be limiting, as at least some granules formed in stromules. This could suggest that stromules form earlier than B-type granules in wild type, or that they form as a result of B-type granule initiation. This is currently difficult to investigate using transmission electron microscopy It is also possible that MRC controls the initiation of stromule formation, thus determining the timing of B-type granule initiation.

MRC as a gene target for biotechnological modification of starch granule size There is significant industrial interest to manipulate starch granule size in crop species, as granule size affects the physico-chemical properties of starch as well as digestibility (Lindeboom et al., 2004; Jobling, 2004). Our results establish MRC as a promising gene target for modifying starch granule size distribution in wheat, specifically to achieve smaller starch granules and a narrower granule size distribution range than conventional cultivars. Small granules are more efficiently digested than large granules, due to their larger surface area to volume ratio (Dhital et al., 2010). B-type granules in particular have a higher rate of water absorption than A-type granules (Chiotelli and Le Meste, 2002). Wheat mrc starch within the food industry include pasta making, where more B-type granules positively affect pasta quality due to their higher rate of water absorption (Soh et al., 2006). They are also desirable for use in papermaking and biodegradable plastics, and as a binder or carrier material in the pharmaceutical and cosmetics industries (Lindeboom et al., 2004; Santelia and Zeeman, 2010). We demonstrated that there is only one functional homeolog of MRC in tetraploid wheat, and likely only two in hexaploid wheat. This is due to the TaMRC 6B becoming a pseudogene, before the more recent second polyploidisation event that created the hexaploidy. The absence of a homeolog makes it more easier to manipulate gene levels in wheat, since less homeologs need to be mutated. Functional tests can be directly performed on our material to provide a proof of concept that the altered granule size distribution in the mutant improves grain/starch quality in these applications.

The repression of B-type granule initiation is likely to be a role specific to those Triticeae species that have a bimodal size distribution of starch granule size in the endosperm. Thus, it remains to be determined what the role of MRC is in cereal species that do not have a bimodal distribution of starch granules, but those that have compound granules (e.g.: in rice). Also, in oats have a bimodal distribution of starch granules, with large compound granules and smaller simple granules. However, in oat, the smaller granules initiate at the same time as the larger compound granules. Thus, it would be interesting to determine whether MRC also has a role in timing the initiation of the small granules during oat endosperm development.

### Materials and Methods

### Plant Materials and Growth

EMS mutants of tetraploid wheat (Triticum turgidum cv. Kronos) carrying mutations in TaMRC 6A and the chromosome 6B pseudogene were identified from the wheat in silico TILLING database (http://www.wheat-tilling.com; Krasileva et al., 2017) and obtained from the John Innes Centre Germplasm Resource Unit. The selected mutants for TaMRC 6A were Kronos3272(K3272), Kronos598(K598) and Kronos4681(K4681); while Kronos4305(K4305) and Kronos3078(K3078) were selected for the 6B pseudogene. From these mutants, we generated three different sets of lines. The mrc-1 lines descend from a cross between K3272 and K3078, while the mrc-2 lines descend from a cross between K4681 and K4305. For both crosses, aa BB, AA bb and aa bb genotypes were obtained in the F2 generation. The mrc-3 lines are uncrossed K598 mutants. The KASP markers used to genotype the mutations are provided in FIG. 9.

For all experiments on grains, plants were grown in soil in a controlled environment room fitted with fluorescent lamps and supplemented with LED panels. The chambers were set to provide a 16-h light/8-h dark cycle, with light intensity of 300 μmol photons m⁻² s⁻¹ and relative humidity of 60%. Temperature was set to 20° C. during the light period, and 16° C. during the dark period. Grains were harvested when the entire spike had senesced and dried (approximately 4 months after sowing). The grains from the first three tillers were used for analysis. For experiments on leaves, plants were grown in a controlled environment chamber set to provide 12-h light/12-h dark cycles and constant temperature (20° C.) and relative humidity (60%).

Starch Purification from Mature Grains or Developing Endosperm

Starch was purified from grains using a method modified from (Peng et al., 1999), using 3-6 grains per extraction. Dry grains were soaked overnight at 4° C. in 5 mL of sterile water. The softened grains were homogenised in 10 mL sterile water using a mortar and pestle, and the homogenate was filtered through a 100 μm mesh. The starch was pelleted by centrifugation at 3,000 g for 5 minutes, and resuspended in 2 mL of water. The resuspended starch was loaded on top of a 5 mL 90% Percoll (Sigma) cushion buffered with 50 mM Tris-HCl, pH 8, and was spun at 2,500 g for 15 minutes. We verified that no intact granules were left in the Percoll interface after the spin. The starch pellet was washed twice times with wash buffer (50 mM Tris-HCl, pH 6.8; 10 mM EDTA; 4% SDS; and 10 mM DTT), then three times with water, followed by a final wash in absolute ethanol. The starch was then air dried overnight.

For starch extraction from developing endosperm, the developing grains were harvested at the indicated timepoints and were frozen until analysis. Each grain was thawed just prior to extraction and the endosperm was carefully dissected and placed into a chilled tube and weighed. The tissue was then homogenised in sterile water with a pestle, then filtered through a 60 μm mesh. The pellet was washed three times in 90% Percoll (Sigma) buffered with 50 mM Tris-HCl, pH 8, then three times with wash buffer (as above), followed by three times with water.

### Coulter Counter Analysis of Starch Granule Size and Number

For profiles of granule size distribution, purified starch was suspended in Isoton II diluent (Beckman Coulter) and analysed with a Multisizer 4e coulter counter fitted with a 70 μm aperture (Beckman Coulter). At least 100,000 granules were counted and sized for the calculation of size distributions. To calculate the mean A- and B-type granule size, as well as relative B-type granule volume, we fitted a mixed bimodal gaussian curve to the distribution using R.

For calculating numbers of granules per grain or in developing endosperm tissue, starch was purified as described above from a known amount of grains or tissue, and was resuspended in a known volume of Isoton II. The coulter counter was used in volumetric mode to determine granule concentrations within a 1.5 mL or 2 mL aliquot of the starch suspension in Isoton II, which was then used to calculate the number of granules in the original grain/endosperm sample.

### Light and Electron Microscopy

For light microscopy of endosperm sections from mature grains, thin sections (1 μm thick) of mature grains were made using a microtome fitted with a glass knife. Sections were mounted onto a glass slide and stained with 3% Lugol's iodine solution (Sigma) prior to imaging.

For light/electron microscopy of developing endosperm tissue, developing grains (15 dpa) were harvested into 4% paraformaldehyde, 2.5% glutaraldehyde in 0.05 M sodium cacodylate, pH 7.4. The osmium post-fixation, dehydration and embedding into LR white resin was done as described above for leaves. For light microscopy, semi-thin sections were stained with toluidine blue, as described for leaves above. For transmission electron microscopy, ultra-thin sections were produced from the embedded grains, and were stained in Uranyl acetate. Imaging was done in a Talos F200C TEM (FEI).

For scanning electron microscopy: For imaging starch granules, a drop of purified starch suspended in water (5 mg/mL) was air-dried onto a glass coverslip attached onto an SEM stub. For imaging sections through developing endosperm, harvested grains were fixed in 2.5% glutaraldehyde in 0.05 M sodium cacodylate, pH 7.4. The fixative was removed by washing with 0.05 M sodium cacodylate, pH 7.4, after which the grains were dehydrated in an ascending ethanol series, and then subjected to critical point drying in a CPD300 instrument (Leica) according to the manufacturer's instructions. Thick transverse sections were produced from the dried grains and were glued onto SEM stubs. All stubs were sputter coated with gold and observed using either a Supra 55 VPFEG (Zeiss) or Nova NanoSEM 450 (FEI) SEM instrument.

### Quantification of Starch Content in Leaves and Endosperm

Starch was quantified in leaf tissue according to Smith and Zeeman (2006). Briefly, frozen leaf tissue was ground into a powder with a ball mill and then extracted with perchloric acid. Starch in the insoluble fraction of the extraction was gelatinised at 95° C., and digested to glucose with α-amylase (Megazyme) and amyloglucosidase (Roche). The glucose released was measured using the hexokinase/glucose-6-phosphate dehydrogenase method (Roche). Starch content (in glucose equivalents) was calculated relative to the original dry weight of the analysed grains.

A similar method to leaves was used for starch quantification in grains. Mature grains (5-6 grains) were soaked overnight at 4° C. in 5 mL of sterile water and were homogenised using a mortar and pestle. Developing endosperm tissue was extracted in 1 mL of sterile water with the pestle. Insoluble material in an aliquot of the homogenate was collected by centrifugation at 5,000 g for 5 mins, then washed once in 0.7 M perchloric acid, once in sterile water, then three times in 80% ethanol. The pellet was then resuspended in water. Starch in the pellet was gelatinised by heating at 95° C. for 15 min, then digested using α-amylase (Megazyme) and amyloglucosidase (Roche).

### Analysis of Amylopectin Structure and Amylose Content

Amylopectin structure and amylose content was analysed using purified starch. Amylopectin structure in terms of chain length distribution was quantified using High Performance Anion Exchange Chromatography with Pulsed Amperometric Detection (HPAEC-PAD). For amylose content, granules were dispersed in DMSO and quantified using an iodine-binding method,

### Example II

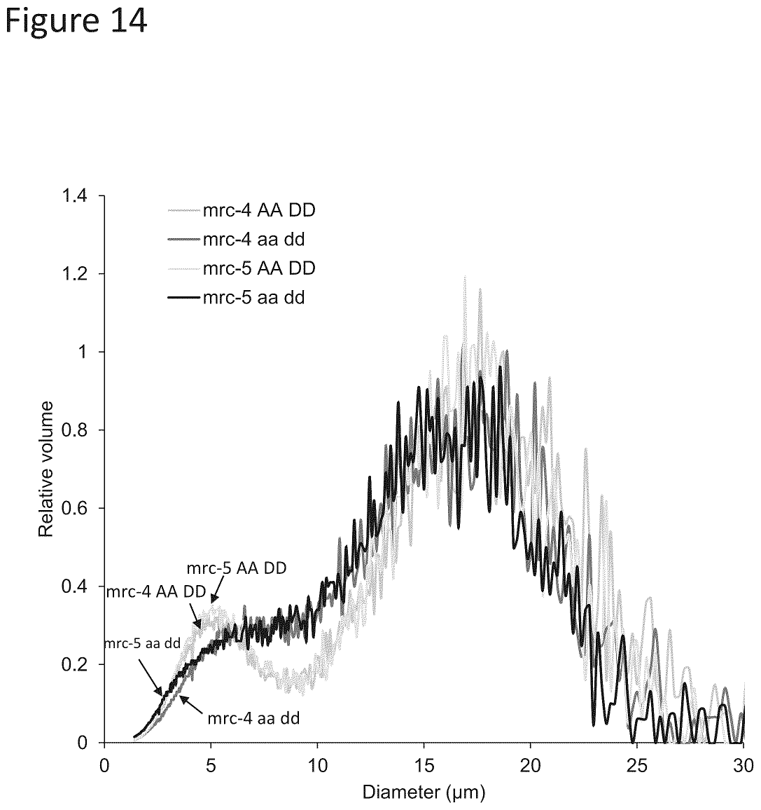

Using the TILLING mutant population in hexaploid wheat (cv. Cadenza) (Krasileva et al., 2017), we have isolated mutants defective in each of the genes encoding the MRC proteins in wheat. The wheat mrc mutants have significantly altered starch granule size distribution in the endosperm (FIG. 14). Unlike Arabidopsis mrc mutants, which produce larger granules, the endosperm starch of the wheat mrc mutants unexpectedly contains more and smaller granules compared to wild type plants. This is due to the production of smaller A-type granules, and more B-type granules. These alterations in granule size distribution also affect gelatinisation temperature—a key parameter in the physico-chemical behaviour of starch. As such starch from the wheat mrc mutant is expected to have applications in the food industry (such as pasta making where more B-type granules positively improve quality; see above reference), applications where the effective digestion of starch is required (biofuels and feed), and in industries where small starch granules are preferred (e.g. papermaking and pharmaceuticals).

Wheat genome sequences (of cultivars Kronos and Cadenza) were obtained from the Grassroots database (https://wheatis.tgac.ac.uk/grassroots-portal/blast; (Clavijo et al., 2017)). This identified that MRC in wheat is encoded on the short arm of chromosome 6A and 6D. Like in Arabidopsis (Seung et al., 2018), the gene has two exons. The genomic DNA sequences of the 6A homeolog (for Kronos and Cadenza) is shown in SEQ ID NO: 4 and 5 respectively, and the 6D (homeolog for Cadenza) is shown in SEQ ID NO: 6. The coding sequences are shown in SEQ ID NOs 7 (6A—Kronos), 8 (6A—Cadenza) and 8 (6D—Cadenza).

Again, no complete homeolog of TaMRC was detected on chromosome 6B. A region with homology to most of exon 2 was detected (sequence provided below), but no region with homology to exon 1 was found. The sequencing data in the region that should contain the first exon was incomplete for both Kronos and Cadenza. However, a complete sequence of the region was available for hexaploid wheat cv. Robigus in the same Grassroots database (Triticum_aestivum_Robigus_Elv1.1_scaffold_041678). Using this scaffold, we determined that there is no exon 1 within a 16 kb region upstream of the start of exon 2. It is thus very unlikely that the part of exon 2 detected on 6B is part of a functioning gene, and the 6B homeolog may have been pseudogenised. This is consistent with the results provided in the following sections, where we show that mutation in the 6B pseudogene has no effect on phenotype.

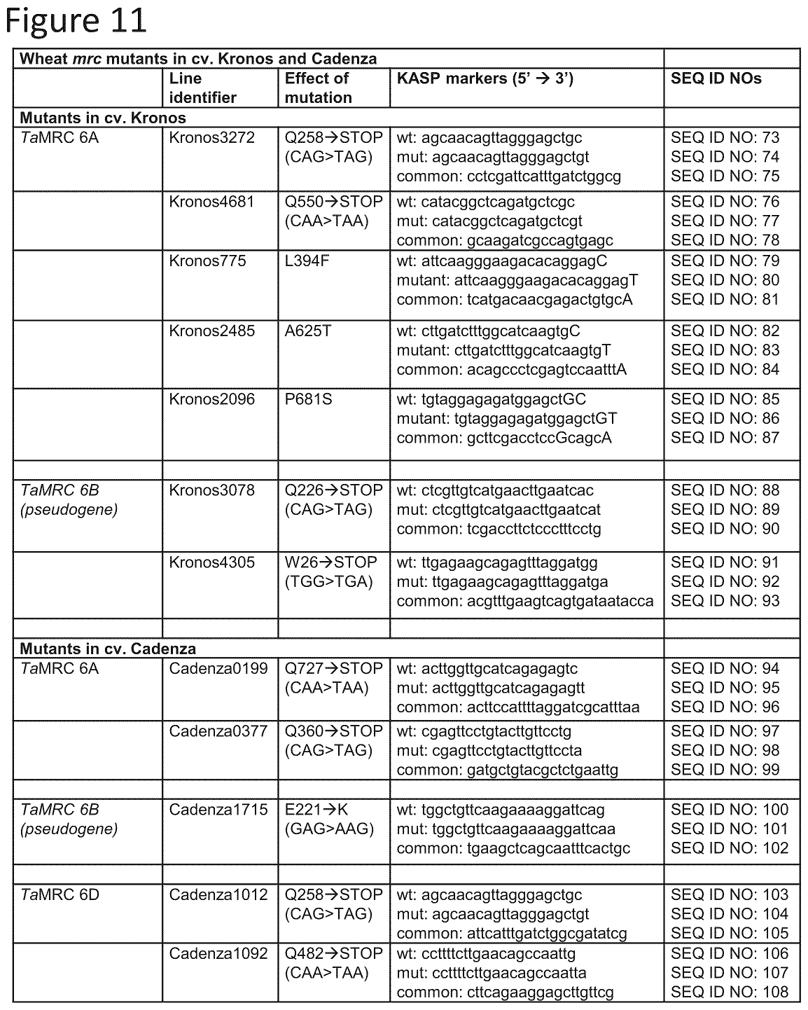

Using the wheat in silico TILLING database (http://www.wheat-tilling.com; Krasileva et al., 2017), we identified EMS-mutagenised Cadenza lines carrying mutations likely to disrupt the function of the TaMRC 6A and 6D homeologs, and the 6B pseudogene. The TILLING database contains exome-capture sequence data from all individuals in the mutant population (Krasileva et al., 2017). Scaffolds corresponding to the TaMRC genes were found by running the BLAST search function of the database using our gene models as the input. We selected lines with mutations that introduce a premature stop codon in the protein coding sequence, as this is most likely to disrupt gene function (FIG. 11). However, for the 6B pseudogene in Cadenza, we selected one missense mutations as premature stop mutations were not available. The effect of each mutation on protein translation is indicated in FIG. 11 (amino acid numbering is based on the translated sequences provided below). KASP markers that were used to identify and genotype these mutations are also provided in FIG. 11.

To combine mutations in multiple homeologs in the same plant, we conducted a series of crosses. In Cadenza, we have crossed the A homeolog mutants with the B homeolog mutants, and individuals from the F1 generation heterozygous for these mutations were crossed with D homeolog mutants. The F1 generation from the latter cross therefore contains individuals that are heterozygous for mutations in all three homeologs. Using this approach, we isolated the following sets:
mrc-4—Cadenza0199×Cadenza1715×Cadenza1012
mrc-5—Cadenza0199×Cadenza1715×Cadenza1092
mrc-6—Cadenza0377×Cadenza1715×Cadenza1012
mrc-7—Cadenza0377×Cadenza1715×Cadenza1092.

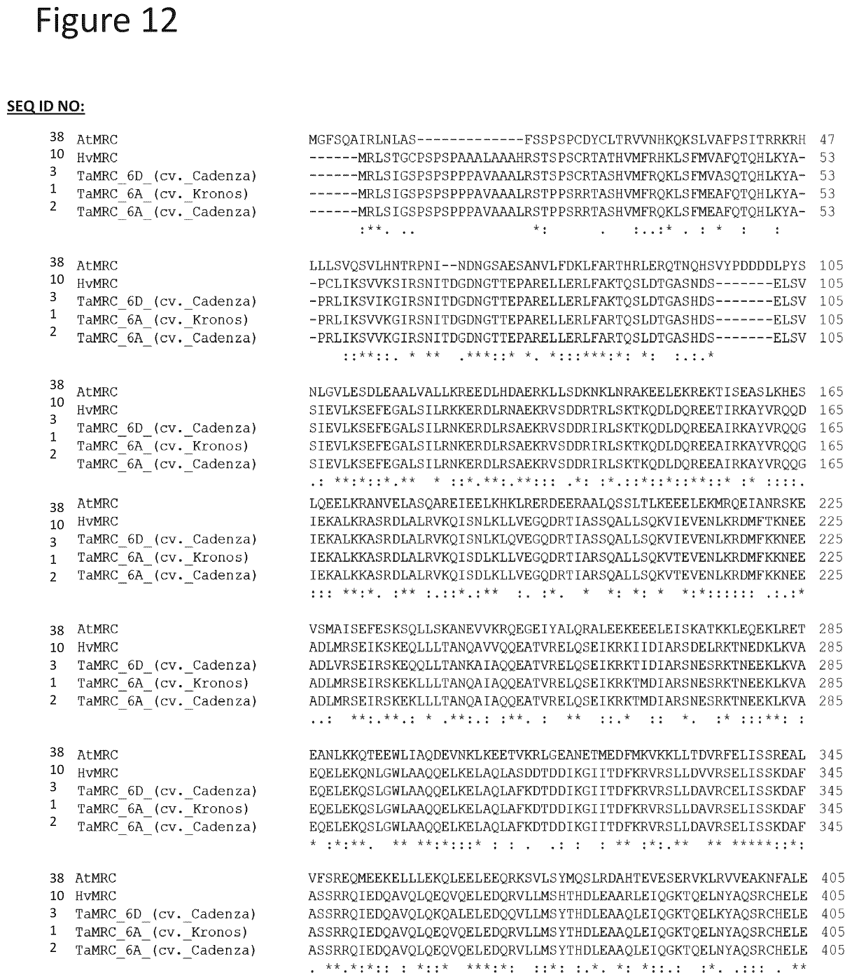

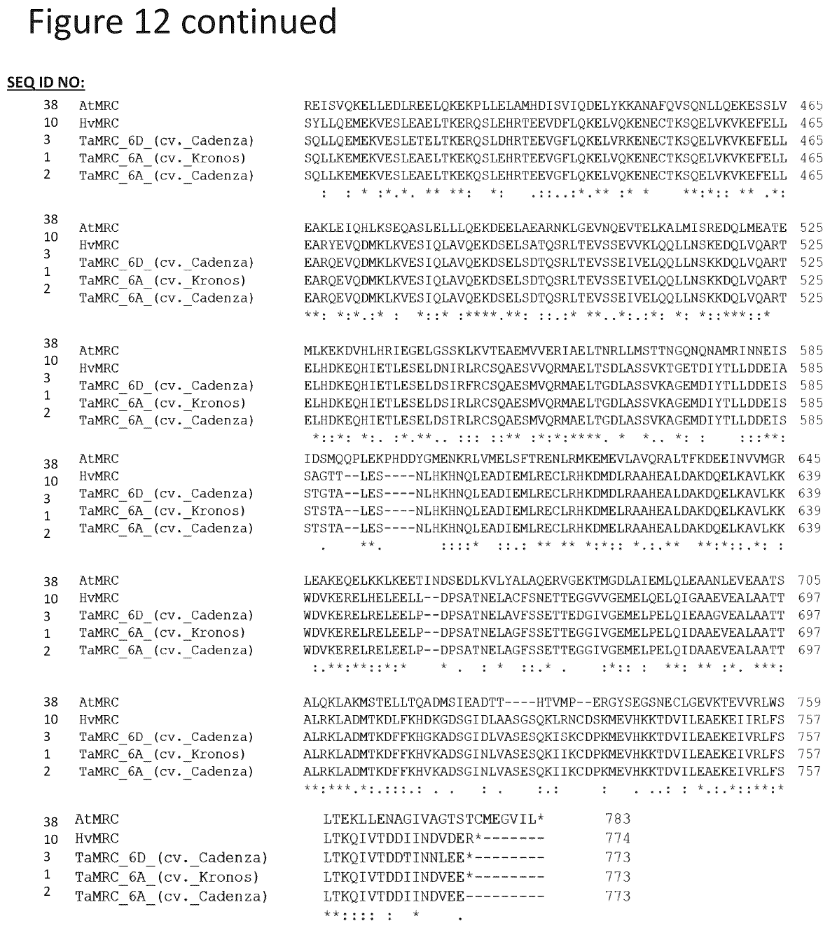

FIG. 12 shows a multiple sequence alignment of AtMRC, HvMRC and TaMRC homeologs from Cadenza and Kronos. The AtMRC and the TaMRC_6A (Cadenza) protein share 33% identity and 58% similarity. Asterisks (*) indicate conserved amino acids, colons (:) indicate highly similar amino acids (according to side chain properties), while point (.) indicate weakly similar amino acids.

Granule size distribution was determined for Cadenza mrc-4 and mrc-5 mutants defective in the A and D homeologs of MRC using a Coulter counter. Two different genotypes were analysed for both mutants—mutants defective in both the 6A and 6D copies of MRC (aa dd), and the negative segregant from the cross (AA DD). The additional mutation of the 6B copy (Cadenza1715) from the cross was segregated out, given that we demonstrated in our Kronos lines that the 6B copy is a pseudogene. As shown in FIG. 14, both aa dd mutants had an altered granule size profile compared to the lines with wild type MRC (AA DD), containing larger B-type granules and smaller A-type granules. The size distribution in these lines most resembled the mrc-2 mutant of Kronos. This is because both mrc-4 and mrc-5 contain a common mutant allele of MRC-6A (Cadenza0199), where the premature stop codon is towards the end of the coding sequence, as in the mrc-2 line of Kronos.

### Example III

Specific mutations can be introduced in the MRC gene using established protocols for CRISPR/Cas9-mediated genome editing in wheat (Shan et al., 2014). The method may involve stable wheat transformation (through tissue culture) to introduce a transgene encoding the single guide RNAs (sgRNAs) targeting the MRC sequence, and encoding the Cas9 enzyme. Regenerated plants containing edits in the MRC gene may be detected in the transformants (the TO generation), and the transgene may be segregated out in the T1 generation. However, a transgene-free approach may also be taken to transiently express the gene-editing components in wheat cells, using the PEG-mediated transformation of wheat protoplasts (Shan et al., 2014) or biolistic bombardment (Zhang et al., 2016; as in the example described below); or biolistic delivery of CRISPR/Cas9 ribonucleoprotein complexes (Liang et al., 2017).

In this example, biolistic transformation of immature wheat embryos allow transient expression of Cas9 with sgRNAs targeting the MRC gene. This is followed by callus induction, and regeneration of edited plants. All steps in this procedure is described in Zhang et al. (2016). First, the appropriate sgRNA (see below) is cloned into the pGE-sgRNA vector (Zhang et al. 2016), which encodes a plant codon-optimised Cas9 driven by the maize Ubiquitin 1 promoter (Wang et al., 2014), and a cloning site for synthetic sgRNA to be synthesised as oligonucleotides and cloned into the vector using the AarI restriction site (Shan et al., 2014). The expression of the sgRNA is driven by the TaU6 promoter. The single pGE-sgRNA vector is then bombarded into isolated wheat embryos. After bombardment, embryos are transferred to callus induction medium. Calli can then be transferred to regeneration medium for plantlet formation. Plantlets are subsequently transferred to rooting medium for shoot formation. Genomic DNA can be extracted from the regenerated plantlets and regions of the MRC gene spanning the sgRNA target site can be amplified using PCR with specific primers. Sequencing the PCR products can reveal plantlets with edited MRC (preferably 1-2 nt deletions or insertions that alter the reading frame for protein translation).

Given the very close similarity in sequence between the 6A and 6D MRC copies, it is possible to produce sgRNAs that target both copies. An ideal sgRNA would be: GCGGCCATGCGCCTCTCCATCGG (SEQ ID NO: 38) where the start codon of the gene is underlined and the Protospacer Adjacent Motif (PAM) is indicated in italics. A BLAST search against the wheat genome shows no off-targets with the same protospacer sequence next to a PAM. This ideal sgRNA would allow edits to occur at or shortly after the start codon, and if it results in a 1-2 nt deletion or addition, would impact the translation of the rest of the protein. Other ideal sgRNAs that target Exon 1, or the first 100 bp of exon 2 include:

(SEQ ID NO: 39)
CAGGCAGAAGCTGAGTTTCATGG
(SEQ ID NO: 40)
ATTAGATCAAATATAACTGATGG
(SEQ ID NO: 41)
AATATAACTGATGGTGATAATGG

All of these sgRNA target both 6A and 6D copies in Cadenza with no mismatches, and have no detectable off-targets. Similarly these sgRNAs would be able to target MRC 6A in Kronos.

### Example IV

We investigated whether the altered granule size distributions in mrc-1 aa bb starch had an impact on the physicochemical properties of starch.

### 1. Gelatinisation

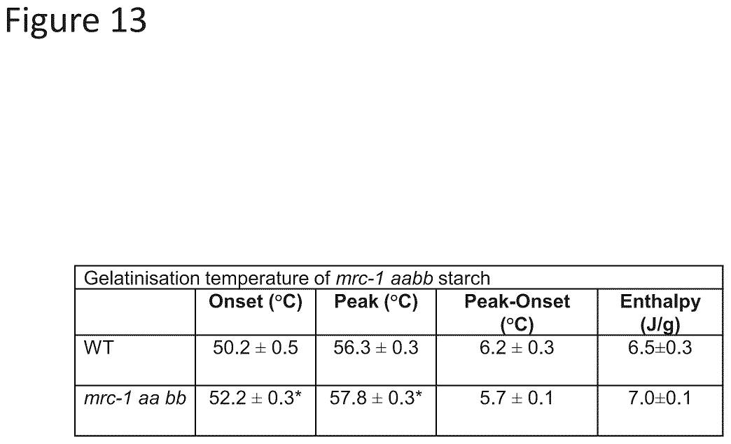

The gelatinisation temperature of starch was examined using Differential Scanning Calorimetry (DSC). Starch (50 mg) was suspended in 1 mL ddH₂O in a DSC pan, and a reference pan contained 1 mL ddH₂O only. Gelatinisation in the sample was monitored in a MC-DSC instrument (TA instruments), heating from 10° C. to 150° C. at a rate of 1° C./min. The mrc-1 aa bb starch had significantly higher onset and peak gelatinisation temperatures compared to the wild type (FIG. 13). The higher peak temperature was expected from the higher proportion of B-type granules in mrc-1 aa bb starch, since it is known that B-type granules have a higher peak gelatinisation temperature than A-type granules (Singh et al., 2003). There was no significant difference between the mutant and wild type was detected for gelatinisation enthalpy, or in the temperature difference between the measured peak and onset temperatures. Thus, mrc-1 aa bb starch has alterations in the physicochemical properties of starch. However, it should be noted that gelatinisation temperature is only one parameter of starch physicochemical behaviour.

### 2. Swelling Power

Swelling power is calculated as the change in starch granule volume after incubation in water at a set temperature. We measured swelling power at both 60° C. and 100° C. Panel A of FIG. 15 shows that the mrc-1 mutant has a swelling power at 100° C. that is almost three-fold higher than the wild-type. Panel B of FIG. 15 shows the change in the average volume of granules over time at 60° C., quantified with a Coulter counter. Starch from mrc-1 swells more and faster than starch from the wild type.

### Method for Calculating Swelling Power

Starch (around 100 mg—precise mass recorded) was added to 10 mL ddH₂O and heated to 60° C. or 100° C. in a water bath for 30 minutes and mixed regularly. Aliquots (250 μL) were collected every 5 minutes and granule size was measured immediately using the Coulter counter. The samples which were used to measure swelling power (as sediment volume change) were removed from the water bath after 30 minutes and left to settle at room temperature for an hour, after which the water was pipetted off to leave only the sediment. The mass of the sediment was then measured, and the swelling power in was calculated using:

$$Swelling power = \frac{{mass}_{sediment}}{{mass}_{initial}}$$

### 3. Viscosity

Purified endosperm starch (2 g) from wild type (wt), mrc-1 or mrc-2 grains was heated in 25 mL of water, and the resulting viscosity was measured on a Rapid Visco Analyser (RVA; Perten Instruments). The mrc-1 starch had an altered viscosity/pasting profile compared to the wild type starch (FIG. 15C). The mrc-2 starch had a similar alteration, that was an intermediate between the wt and mrc-1. This suggests that altered starch granule size distributions in the mrc mutants can modify the viscocity during gelatinisation and pasting.

### Example V

### Conversion of Starch to Glucose During Germination

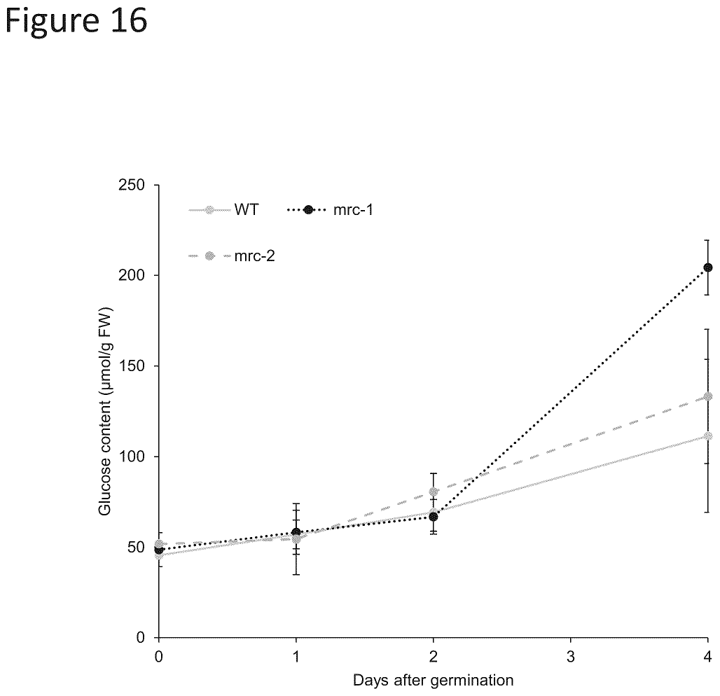

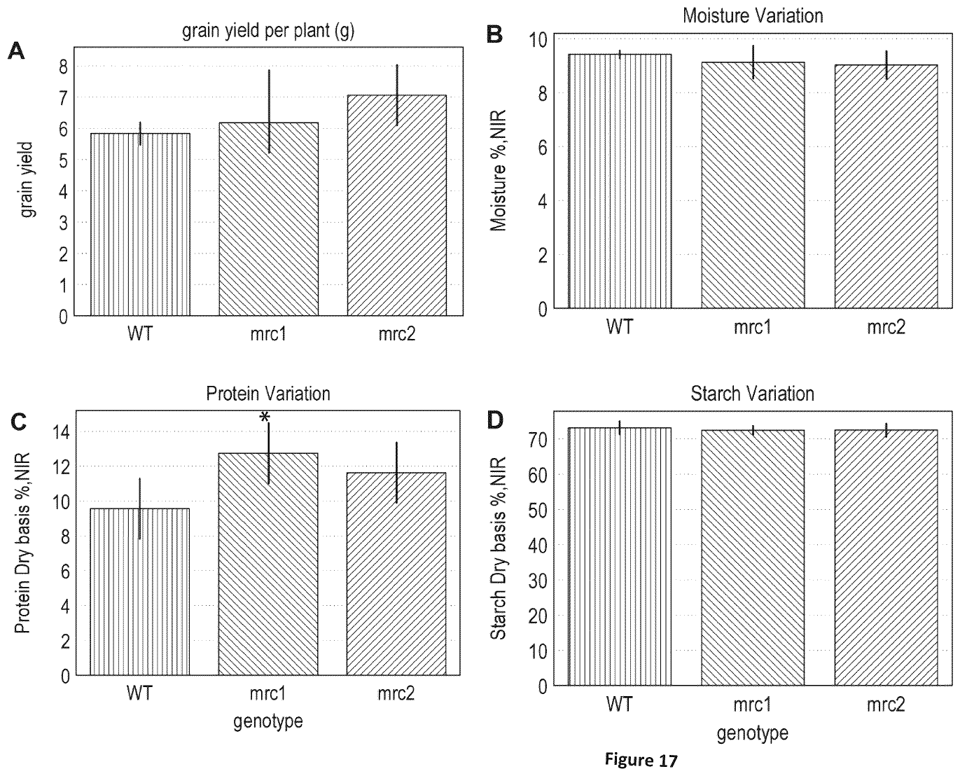

We tested whether grains of the mrc mutants can convert starch to simple sugars more efficiently during germination than the wild type. α-Amylase is synthesised de novo in the aleurone layer within the first two days of germination and is secreted into the endosperm. α-Amylase initiates the digestion of starch, which is eventually degraded to glucose. The overall decrease in granule size in the mutant could allow for a higher starch digestion rate, due to an increase in surface area available for germination. Thus, we measured glucose accumulation in the endosperm at the onset of starch degradation. As shown in FIG. 16, four days into germination, grains of the mrc-1 mutant accumulated significantly more glucose than the wild type and the mrc-2 mutant grains, under a two-tailed t-test (p<0.05).

This process mimics malting, which involves the controlled germination of grains for 4-6 days, and suggests that malt from the mrc mutant grains will have more starting sugars available for fermentation. The more efficient breakdown of starch in vivo may also translate to the later stages of brewing, where the remaining starch in the malted grain is digested during mashing.

### Methods

Grains were germinated on damp filter paper in petri dishes in the dark at 20° C. At each timepoint, three replicate grain samples each containing three grains, were flash frozen in liquid nitrogen. The embryo/sprout was removed from the grain prior to collection. Sugars were then extracted from the grains by homogenising in 0.7 M perchloric acid using a ball mill. Insoluble material was removed, and the supernatant was neutralised using 2 M KOH, 400 mM MES. Glucose was assayed using the hexokinase/glucose-6-phosphate dehydrogenase method (Roche)

### Example VI

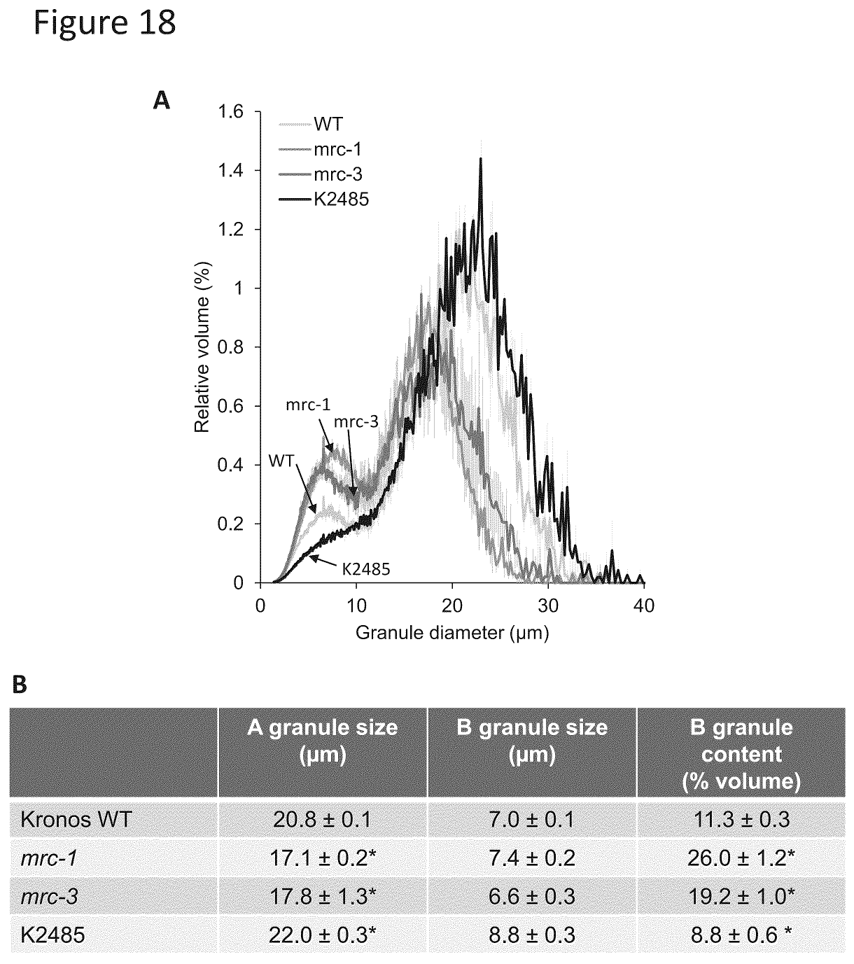

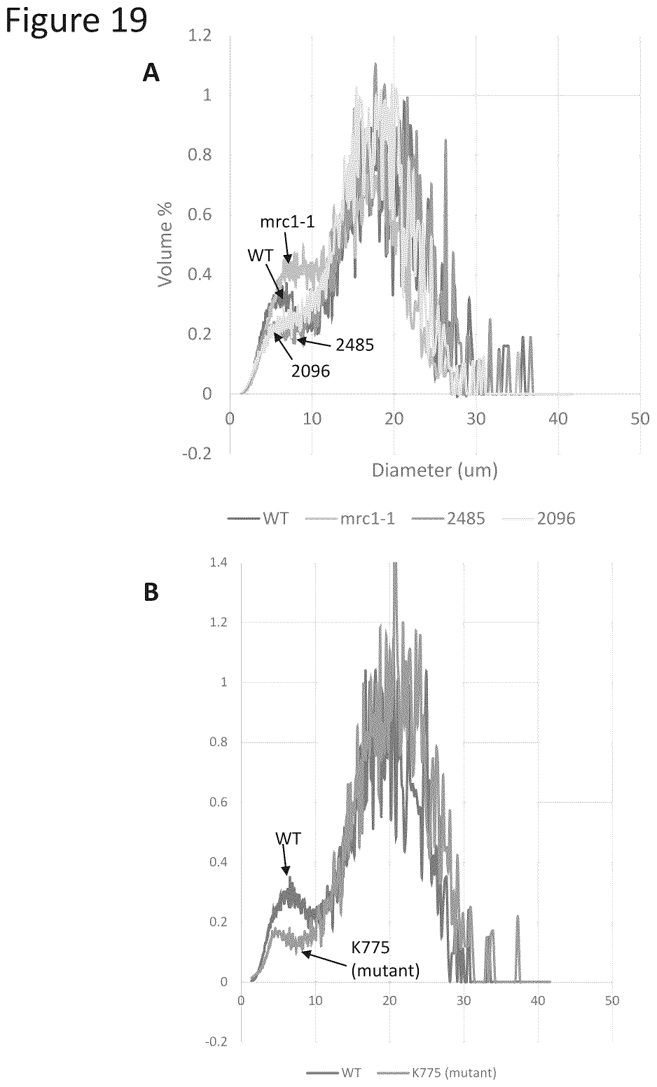

Kronos2485 (K2485) has the opposite phenotype of mrc-1, mrc-2 and mrc-3, in that it has larger A-type granules and fewer B-type granules than the wild type. Kronos2485 was discovered in an experiment that aimed to use the wheat TILLING mutant resource (described above) to discover mutants carrying amino acid substitutions in MRC that alter granule size distribution. Lines with substitutions in otherwise conserved amino acid positions were obtained from the resource. Starch was extracted from grains from these lines and analysed on a Coulter counter (as described for example 1). Kronos2485 contained significantly fewer B-type granules (FIG. 18). This was accompanied by an increase in A-type granule size. This changes in granule size distribution are opposite to that of mrc-1, suggesting that the amino acid substitution (A625T) is an activating or gain-of-function mutation. MRC protein containing this substitution is likely to over-suppress B-type granule formation, leading to fewer B-type granules. This would provide more substrates (i.e.: ADP-Glucose) for A-type granule growth during grain development, leading to the larger A-type granules.

During this experiment, two more lines with a similar granule size distribution to Kronos2485 were discovered. Kronos2096 (containing a P681S substitution) and Kronos775 (containing a L394F substitution) both had fewer B-type granules than the wild type.

These results demonstrate that depending on the type of MRC mutation, granule size distributions can be altered in opposite directions (smaller or larger). In species with bimodal granule size distributions like wheat, the reduction in B-type granule content induced by these activating mutations achieve an overall reduction in bimodality. Thus, starch from these lines may have some of benefits of starch with a unimodal granule distribution, described above.

### Example VII

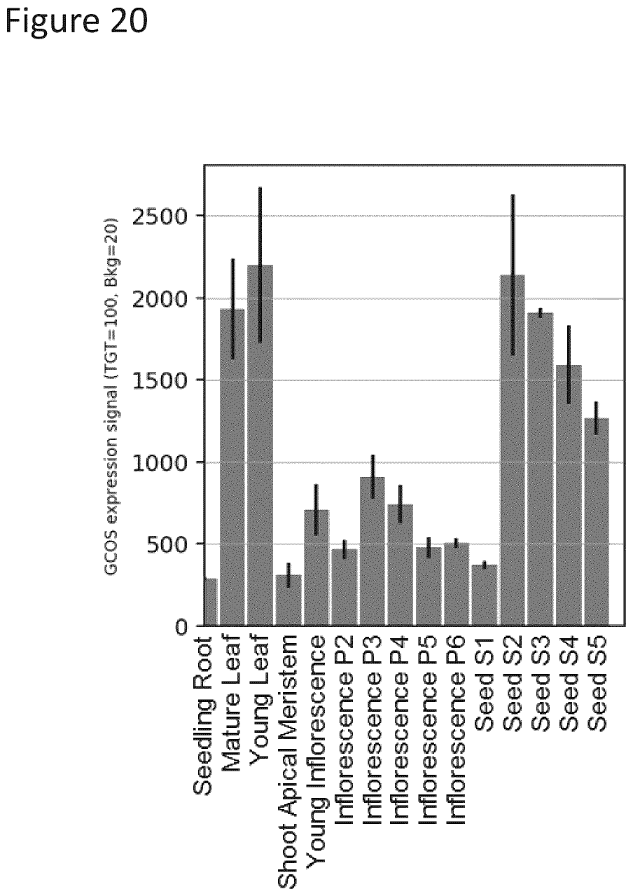

To briefly investigate the potential of using MRC to introduce similar changes in starch granule size distribution in other cereal crops, we checked a public gene expression database to determine if MRC was also expressed in developing rice grains. The gene was robustly expressed in all stages of seed development, particularly at 3-4 dpa (FIG. 20), when starch granules initiate in rice (Matsushima et al., 2015). This hints that the role of MRC in endosperm starch synthesis may be conserved among cereals.

### Example VIII

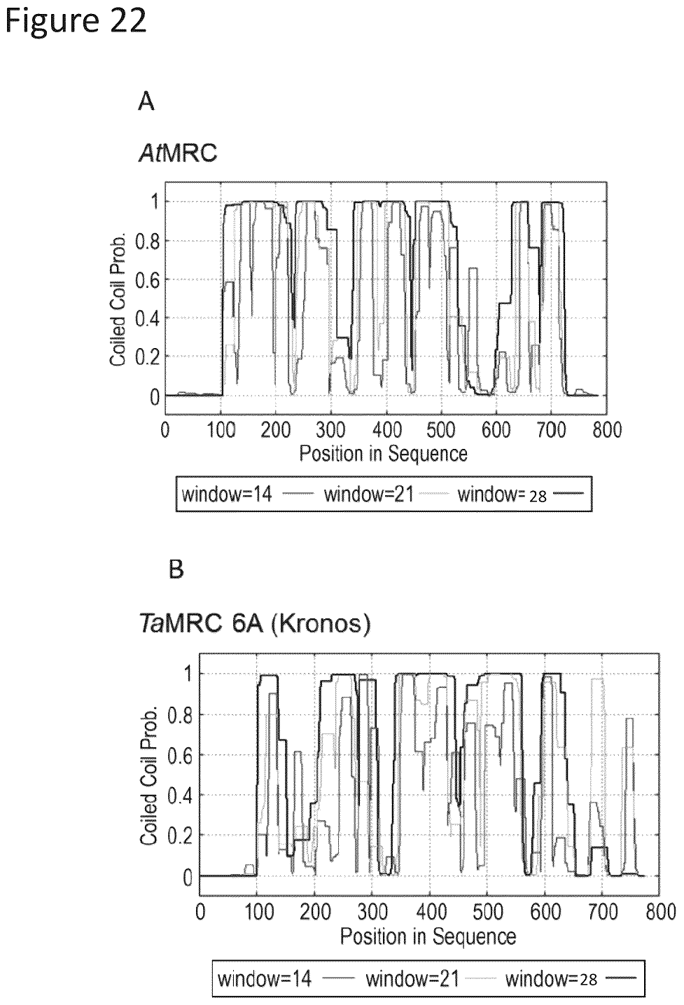

Given that the AtMRC protein forms extensive coiled coils along almost the entire length of the protein (Seung et al., 2018), we used the COILS program (Lupas, 1995; Zimmermann et al., 2017) to predict whether the wheat orthologs also contain coiled coils. Very similar patterns of strong coiled coil predictions were obtained for the AtMRC and TaMRC (6A) proteins (FIG. 22). Thus, TaMRC is also a long coiled coil protein. The exact role of these coiled coils is not known, but since coiled coils can mediate protein-protein interaction with other coiled coil-containing proteins, and AtMRC interacts with AtSS4 (which also contains coiled coils), it is likely that at least some of these coiled coils in MRC mediate an interaction with SS4.

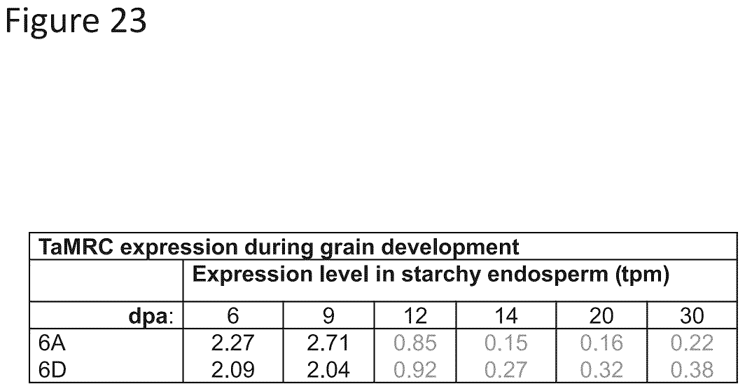

To investigate whether TaMRC could play a role in endosperm starch formation, we used the wheat expression browser to check whether the gene is expressed in the starchy endosperm during grain development (Borrill et al., 2016) (FIG. 23). The 6A and 6D homeologs showed similar levels and patterns of expression, with expression higher in the early stages of grain development (6-9 dpa) than the later stages (12-30 dpa). Interestingly, this peak in expression during early grain development corresponds to when the synthesis of the A-type granules initiate.

To investigate whether the size distribution of endosperm starch was affected by the absence of TaMRC, we isolated starch from the endosperm of the mrc-1 and mrc-2 mutants. The mutants and wild type plants were grown in soil (1 L capacity pots) in a controlled environment room (Conviron) fitted with fluorescent lamps and supplemented with LED panels. The chambers were set to provide a 16-h light/8-h dark cycle, with light intensity of 300 μmol photons m⁻² s⁻¹ and humidity of 60%. Temperature was set to 20° C. during the light period, and 16° C. during the dark period. Grains were harvested when the entire spike had senesced and dried. The grains from the middle of the first three spikes were used for analysis.

Starch was isolated from 6 grains per biological replicate (each replicate being an individual plant). The dry grains were soaked overnight at 4° C. in 5 mL of sterile ddH₂O. The softened grains were homogenised in 10 mL sterile ddH₂O using a mortar and pestle, and the homogenate was filtered through a 100 μm mesh. The starch was pelleted by centrifugation at 4,000 g for 5 minutes, and then resuspended in 2 mL of water. The resuspended starch was loaded on top of a 90% Percoll cushion (5 mL; Sigma), and was spun at 2,500 g for 10 minutes. The starch pellet was washed three times with wash buffer (50 mM Tris-HCl, pH 6.8; 10 mM EDTA; 4% SDS; and 10 mM DTT), then three times with water, followed by a final wash in absolute ethanol. The starch was then air dried overnight.

The starch granule morphology was then observed by Scanning Electron Microscopy (SEM). The purified starch was resuspended in water (5 mg/mL) and 2 μL was applied to a SEM stub. The starch was air dried overnight on the stub before sputter coating with gold, and was observed using a Supra 55 VP FEG SEM (Zeiss).

The starch granules from the mrc-1 aa bb mutant was noticeably different in size from the wild-type WT Kronos (AA BB) starch (FIG. 2B). The starch in the mutant appeared to contain more B-type granules than the starch from the wild type, and the A-type granules were noticeably smaller in the mutant. The mrc-2 aa bb mutant also had smaller granules than the wild type, but to a lesser extent than mrc-1 aa bb. Aside from the difference in size, no other morphological alterations were observed. In both WT and mutant starch, the A-type granules had their typical lenticular shape, while the B-type granules were round.

Since qualitative alterations in granule size distribution were observed in the mrc mutant starch with SEM, we then quantitatively measured granule size using two different methods. Firstly, we used a particle size analyser, which uses laser scattering to measure the total volume of particles of a given size, expressed as a percentage of the total volume of all particles. Purified starch was suspended in water and measured on the Coulter LS-230 instrument (Beckman Coulter). In WT starch, a clear bimodal distribution was observed, with an A-type granule peak around 28 μm, and a B-type granule peak around 5 μm (FIG. 2C). However, the starch from mrc-1 aa bb mutants had a highly altered size profile compared to the wild type—the A-type granule peak was shifted to 21 μm, and the B-type granule peak was higher and shifted towards the larger sizes, appearing as a peak at 8 μm which was not completely resolved from the A-type granule peak. Since the A-type granules are smaller in the mutant, and B-type granules are larger, the profile more closely resembled unimodal distribution of granule size. Similar changes were observed in mrc-2 aa bb mutants, although the phenotype was not as strong as in mrc-1 aa bb.

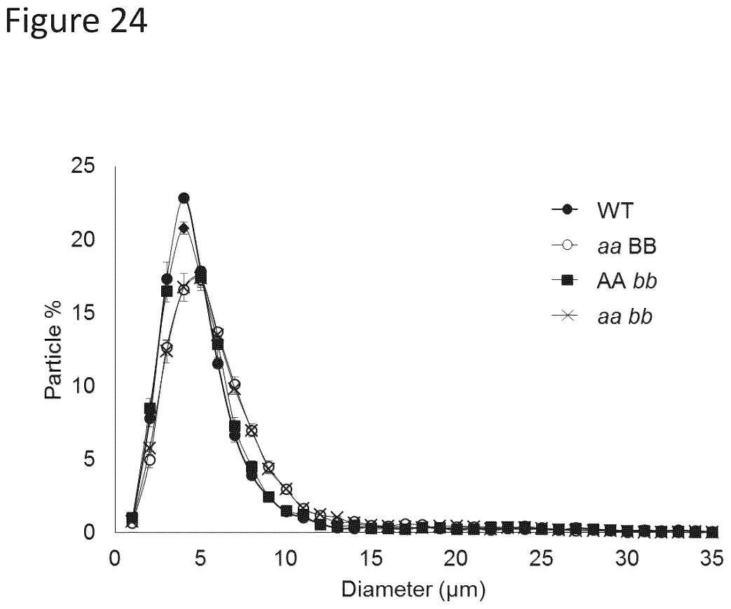

We also analysed starch granule size using light microscopy. Purified starch was resuspended at 5 mg/mL in water, and 2 μL of the suspension was added to 100 uL 10% Lugol's iodine solution (Sigma) in a well of a microtitre plate. The starch in the well was imaged using an AxioObserver microscope (Zeiss). Starch granule area in the images was measured using the Particle Analysis plugin of ImageJ software (v.2.0.0; https://imagej.net/). The area was used to calculate diameter, assuming the granules were perfect circles. Unlike the first method with the particle size analyser, this method calculates of the percentage of granules with a given size relative to the total number of granules (rather than as volumes), and is a direct measure of size (rather than inferred from laser scattering). This analysis is not able to measure A-type granule size distribution accurately, as they constitute less than 10% of the total number of granules. However, B-type granule size can be measured accurately. The mrc-1 aa bb mutant had a clear increase in B-type granule size relative to the wild type, with fewer granules in the 1-5 μm size range, and more granules in the 6-13 μm size range (FIG. 24). Interestingly, exactly the same B-type granule size profile was obtained between mrc-1 aa bb and mrc-1 aa BB, suggesting that the mutation in the A homeolog of Kronos alone is sufficient to observe the phenotype. This is consistent with the hypothesis that the B genome copy is a pseudogene.

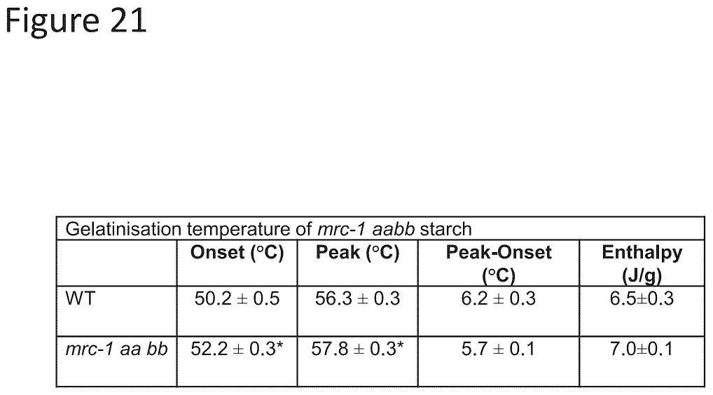

We investigated whether the altered granule size distributions in mrc-1 aa bb starch had an impact on the gelatinisation temperature of starch using Differential Scanning Calorimetry (DSC). Starch (50 mg) was suspended in 1 mL ddH₂O in a DSC pan, and a reference pan contained 1 mL ddH₂O only. Gelatinisation in the sample was monitored in a MC-DSC instrument (TA instruments), heating from 10° C. to 150° C. at a rate of 1° C./min. The mrc-1 aa bb starch had significantly higher onset and peak gelatinisation temperatures compared to the wild type (FIG. 21). The higher peak temperature was expected from the higher proportion of B-type granules in mrc-1 aa bb starch, since it is known that B-type granules have a higher peak gelatinisation temperature than A-type granules (Singh et al., 2003). No significant difference between the mutant and wild type was detected for gelatinisation enthalpy, or in the temperature difference between the measured peak and onset temperatures. Thus, mrc-1 aa bb starch has alterations in the physicochemical properties of starch. However, it should be noted that gelatinisation temperature is only one parameter of starch physicochemical behaviour.

To briefly investigate the potential of using MRC to introduce similar changes in starch granule size distribution in other cereal crops, we checked a public gene expression database to determine if MRC was also expressed in developing rice grains. The gene was robustly expressed in all stages of seed development, particularly at 3-4 dpa (FIG. 20), when starch granules initiate in rice (Matsushima et al., 2015). This hints that the role of MRC in endosperm starch synthesis may be conserved among cereals.

The mrc-1 and mrc-2 aa bb mutants were indistinguishable from wild type Kronos plants in terms of plant size and morphology, as well as tiller number and flowering time. The mature grains from the mutants were similar in size and morphology to wild-type grains.

To verify that the mrc mutations did not affect the amount of starch in the mature grains, we measured grain total starch content. The mature grains (5-6 grains) were soaked overnight at 4° C. in 5 mL of sterile ddH₂O, and were homogenised using a mortar and pestle. Insoluble material in the homogenate was collected by centrifugation at 5,000 g for 5 mins, then washed once in 0.7 M perchloric acid, once in ddH₂O, then three times in 80% EtOH. The pellet was then resuspended in ddH₂O. Starch in the pellet was gelatinised by heating at 95° C. for 15 min, then digested using α-amylase (Megazyme) and amyloglucosidase (Roche). The glucose released was measured using the hexokinase/glucose-6-phosphate dehydrogenase method (Roche). Starch content (in glucose equivalents) was calculated relative to the original dry weight of the analysed grains.

No changes in total starch content was observed in any of the mrc-1 mutants (FIG. 10). This suggests that the mutation can alter the size distribution of granules in the endosperm with no negative impact on the total amount of starch synthesised.

### REFERENCES

Bechtel D B, Zayas I, Kaleikau L, Pomeranz Y. 1990. Size-distribution of wheat starch granules during endosperm development. Cereal Chemistry 67, 59-63.
Borrill P, Ramirez-Gonzalez R, Uauy C. 2016. expVIP: a customisable RNA-seq data analysis and visualisation platform. Plant Physiology 170, pp. 01667.2015.
Cermak, T et al. Efficient design and assembly of custom TALEN and other TAL effector-based constructs for DNA targeting. Nucleic acid Res. 39 (2011).
Chia, T.; Adamski, N. M.; Saccomanno, B.; Greenland, A.; Nash, A.; Uauy, C.; Trafford, K.; Lunn, J. 2017 Transfer of a starch phenotype from wild wheat to bread wheat by deletion of a locus controlling B-type starch granule content. J. Exp. Bot.
Chia T, Chirico M, King R, Ramirez-Gonzalez R, Saccomanno B, Seung D, Simmonds J, Trick M, Uauy C, Verhoeven T, Trafford K. 2019. A carbohydrate-binding protein, FLOURY ENDOSPERM 6 influences the initiation of A- and B-type starch granules in wheat (Preprint)
Chiotelli, E. and Le Meste, M. (2002). Effect of Small and Large Wheat StarchGranules on Thermomechanical Behavior of Starch. Cereal Chem. 79: 286-293.
Clavijo B J, Venturini L, Schudoma C, et al. 2017. An improved assembly and annotation of the allohexaploid wheat genome identifies complete families of agronomic genes and provides genomic evidence for chromosomal translocations. Genome Research 27, 885-896.
Clough S J, Bent A F. 1998. Floral dip: a simplified method for Agrobacterium-mediated transformation of Arabidopsis thaliana. Plant J. 16(6): 735-43.
Comai L, Young K, Till B J, Reynolds S H, Greene E A, Codoma C A, Enns L C, Johnson J E, Burtner C, Odden A R, Heinkoff. 2004. Efficient discovery of DNA polymorphisms in natural populations by Ecotilling. Plant J. 37(5):778-86.
Crumpton-Taylor M, Grandison S, Png K M Y, Bushby A J, Smith A M. 2012. Control of starch granule numbers in Arabidopsis chloroplasts. Plant Physiology 158, 905-916.
Crumpton-Taylor M, Pike M, Lu K J, Hylton C M, Feil R, Eicke S, Lunn J E, Zeeman S C, Smith A M. 2013. Starch synthase 4 is essential for coordination of starch granule formation with chloroplast division during Arabidopsis leaf expansion. New Phytologist 200, 1064-1075.
Dhital S, Shrestha A K, Gidley M J. 2010. Relationship between granule size and in vitro digestibility of maize and potato starches. Carbohydrate Polymers 82, 480-488.
Emanuelsson O, Brunak S, von Heijne G, Nielsen H. 2007. Locating proteins in the cell using TargetP, SignalP and related tools. Nature protocols 2, 953-971.
Goren A, Ashlock D, Tetlow I J. 2018. Starch formation inside plastids of higher plants. Protoplasma, 1-22.
Henikoff S, Till B J, Comai L. 2004. TILLING. Traditional mutagenesis meets functional genomics. Plant Physiol. 135(2): 630-6.
Howard T, Rejab N A, Griffiths S, Leigh F, Leverington-Waite M, Simmonds J, Uauy C, Trafford K. 2011. Identification of a major QTL controlling the content of B-type starch granules in Aegilops. Journal of Experimental Botany 62, 2217-2228.
Jane J F. 1994. Anthology of Starch Granule Morphology by Scanning Electron Microscopy. Stach/Starke 46, 121-129.
Jane J, Chen Y Y, Lee L F, Mcpherson A E, Wong K S, Radosavljevic M, Kasemsuwan T. 1999. Effects of amylopectin branch chain length and amylose content on the gelatinization and pasting properties of starch. Cereal Chemistry 76, 629-637.
Jobling S. 2004. Improving starch for food and industrial applications. Current Opinion 50 in Plant Biology 7, 210-218.
Komor, A. C.; Kim, Y. B.; Packer, M. S.; Zuris, J. A.; Liu, D. R. 2016. Programmable editing of a target base in genomic DNA without double-stranded DNA cleavage. Nature 533, 420-424
Krasileva K V., Vasquez-Gross H A, Howell T, et al. 2017. Uncovering hidden variation in polyploid wheat. Proceedings of the National Academy of Sciences 114, E913-E921.
Krysan P J, Young J C, Sussman M R. 1999. T-DNA as an insertional mutagen in Arabidopsis. Plant Cell. 11(12): 2283-90.
Kunkel T A. 1985. Rapid and efficient dite-specifc mutagenesis without phenotypic selection. PNAS. 82(2): 488-92.
Kunkel T A, Roberts J D, Zakour R A. 1987. Rapid and efficient dite-specifc mutagenesis without phenotypic selection. Methods Enzmol. 154. 367-82.
Langeveld S M J, Van wijk R, Stuurman N, Kijne J W, de Pater S. 2000. B-type granule containing protrusions and interconnections between amyloplasts in developing wheat endosperm revealed by transmission electron microscopy and GFP expression. Journal of Experimental Botany 51, 1357-1361.
Liang Z, Chen K, Li T, et al. 2017. Efficient DNA-free genome editing of bread wheat using CRISPR/Cas9 ribonucleoprotein complexes. Nature Communications 8, 1-5.
Lindeboom N, Chang P R, Tyler R T. 2004. Analytical, biochemical and physicochemical aspects of starch granule size, with emphasis on small granule starches: a review. Starch Starke 56, 89-99.
Lupas A. 1995. Prediction and analysis of coiled-coil structures. Methods in Enzymology 266, 513-525.
Ma X, Zhang Q, Zhu Q, Liu W, Chen Y, Qiu R, Wang B, Yang Z, Li H, Lin Y, Xie Y, Shen R, Chen S, Wang Z, Chen Y, Guo J, Chen L, Zhao X, Dong Z, Liu Y. 2015. A Robust CRISPR/Cas9 System for Convenient, High-Efficiency Multiplex Genome Editing in Monocot and Dicot Plants. 8(8): 1274-84.
Mason J M, Arndt K M. 2004. Coiled coil domains: stability, specificity, and biological implications. Chembiochem: a European journal of chemical biology 5, 170-176.
Matsushima R, Maekawa M, Sakamoto W. 2015. Geometrical Formation of Compound Starch Grains in Rice Implements Voronoi Diagram. Plant and Cell Physiology 56, 2150-2157.
Ng P C, Henikoff S. Predicting the effects of amino acid substitutions on protein function. Annu Rev Genomics Hum Genet. 2006; 7:61-80. doi: 10.1146/annurev.genom.7.080505.115630
Parker M L. 1985. The relationship between A-type and B-type starch granules in the developing endosperm of wheat. Journal of Cereal Science 3,271-278.
Peng, M., Gao, M., Abdel-Aal, E-S. M., Hucl, P., Chibbar, R. N., 1999. Separation and characterization of A- and B-type starch granules in wheat endosperm. Cereal Chem. 76, 375-379.
Pfister B, Zeeman S C. 2016. Formation of starch in plant cells. Cellular and Molecular Life Sciences.
Roldán I, Wattebled F, Mercedes Lucas M, Delvallé D, Planchot V, Jiménez S, Pérez R, Ball S, D'Hulst C, Mérida A. 2007. The phenotype of soluble starch synthase IV defective mutants of Arabidopsis thaliana suggests a novel function of elongation enzymes in the control of starch granule formation. Plant Journal 49, 492-504.
Radhar M, McMahon M A, Prakash T P, Swayze E E, Bennett F, Cleveland D W. 2015. Synthetic CRISPR RNA-Cas9-guided genome editing in human cells. PNAS. 112(51) E7110-E7117.
Neville E Sanjana, Le Cong, Yang Zhou, Margaret M Cunniff, Guoping Feng& Feng Zhang. 2012. A transcription activator-like effector toolbox for genome engineering, Nature Protocols 7, 171-192.
Sambrook, et al., (1989) Molecular Cloning: A Library Manual (2d ed., Cold Spring Harbor Laboratory Press, Plainview, New York).
Santelia D, Zeeman S C. 2010. Progress in Arabidopsis starch research and potential biotechnological applications. Current Opinion in Biotechnology 22, 271-280.
Seung D, Boudet J, Monroe J D, Schreier T B, David L C, Abt M, Lu K-J, Zanella M, Zeeman S C. 2017. Homologs of PROTEIN TARGETING TO STARCH control starch granule initiation in Arabidopsis leaves. The Plant Cell 29, 1657-1677.
Seung D, Schreier T, Burgy L, Eicke S, Zeeman S. 2018. Two plastidial coiled-coil proteins are essential for normal starch granule initiation in Arabidopsis. Plant Cell, In press.
Seung D, Soyk S, Coiro M, Maier B A, Eicke S, Zeeman S C. 2015. PROTEIN TARGETING TO STARCH is required for localising GRANULE-BOUND STARCH SYNTHASE to starch granules and for normal amylose synthesis in Arabidopsis. PLoS Biology 13, e1002080.
Shan Q, Wang Y, Li J, Gao C. 2014. Genome editing in rice and wheat using the CRISPR/Cas system. Nature Protocols 9, 2395-2410.
Singh N, Singh J, Kaur L, Sodhi N S, Gill B S. 2003. Morphological, thermal and rheological properties of starches from different botanical sources. Food Chemistry 81, 219-231.
Smith A M. 2008. Prospects for increasing starch and sucrose yields for bioethanol production. Plant Journal 54, 546-558.
Soh H N, Sissons M J, Turner M A. 2006. Effect of Starch Granule Size Distribution and Elevated Amylose Content on Durum Dough Rheology and Spaghetti Cooking Quality. Cereal Chemistry 83, 513-519.
Stoddard F L. Survey of starch particle-size distribution in wheat and related species, Cereal Chemistry, 1999, vol. 76 (pg. 145-149)
Stoddard F L, Sarker R. Characterization of starch in Aegilops species, Cereal Chemistry, 2000, vol. 77 (pg. 445-447)
Vandromme C, Spriet C, Dauvill D, Courseaux A, Putaux J, Wychowski A, Facon M, D'Hulst C, Wattebled F. 2018. PII1: a protein involved in starch initiation that determines granule number and size in Arabidopsis chloroplast. New Phytologist (online; https://doi.org/10.1111/nph.15356).
Wang Y, Cheng X, Shan Q, Zhang Y, Liu J, Gao C, Qiu J L. 2014. Simultaneous editing of three homoeoalleles in hexaploid bread wheat confers heritable resistance to powdery mildew. Nature Biotechnology 32, 947-951.
Wiles M V, Qin W, Cheng A W, Wang H (2015) CRISPR-Cas9-mediated genome editing and guide RNA design. Mamm Genome 26:501-510
Zeeman S C, Kossmann J, Smith A M. 2010. Starch: its metabolism, evolution, and biotechnological modification in plants. Annual Review of Plant Biology 61, 209-234.
Zeeman S C, Tiessen A, Pilling E, Kato K L, Donald A M, Smith A M. 2002. Starch synthesis in Arabidopsis. Granule synthesis, composition, and structure. Plant Physiology 129, 516-529.
Zhang Y, Liang Z, Zong Y, Wang Y, Liu J, Chen K, Qiu J L, Gao C. 2016. Efficient and transgene-free genome editing in wheat through transient expression of CRISPR/Cas9 DNA or RNA. Nature Communications 7, 1-8.
Zimmermann L, Stephens A, Nam S Z, Rau D, Kübler J, Lozajic M, Gabler F, Soding J, Lupas A N, Alva V. 2017. A Completely Reimplemented MPI Bioinformatics Toolkit with a New HHpred Server at its Core. Journal of Molecular Biology 430, 2237-2243.

1. A method for altering starch granule size distribution in a plant, the method comprising altering the expression of at least one MRC (MYOSIN-RESEMBLING CHLOROPLAST PROTEIN) nucleic acid and/or altering the activity of a MRC polypeptide, wherein the plant is not Arabidopsis.

2. The method of claim 1, wherein altering the granule size distribution comprises shifting the granule size distribution towards larger granules, and wherein the method comprises introducing at least one mutation into at least one MRC nucleic acid sequence encoding an MRC polypeptide wherein the mutation is a gain of function mutation.

3. A genetically altered plant, part thereof or plant cell, wherein said plant, part thereof or plant cell is characterized by an altered starch granule size distribution, within a grain or seed compared to a control or wild-type grain or seed, wherein the altered granule size distribution is a shift in granule size distribution towards larger granules, and wherein the plant grain or seed has increased expression of at least one MYOSIN-RESEMBLING CHLOROPLAST PROTEIN (MRC) nucleic acid and/or increased activity of a MRC polypeptide compared to a wild-type or control grain or seed wherein the plant grain or seed comprises at least one mutation in at least one nucleic acid encoding a MRC polypeptide, and wherein the mutation is a gain of function mutation, and wherein the plant is not Arabidopsis.

4. The genetically altered plant, part thereof or plant cell of claim 3, wherein the nucleic acid sequence encodes a MRC polypeptide as defined in one of SEQ ID NO: 1 to 3 or 29 to 30 or a functional variant or homolog thereof, wherein the functional variant or homolog has at least 80% overall sequence identity to SEQ ID NO: 1 to 3 or 29 to 30.

5. The genetically altered plant, part thereof or plant cell of claim 3, wherein the plant, part thereof or plant cell of is selected from wheat, barley, rye, maize, potato, sorghum and rice.

6. The plant part of claim 3, wherein said plant part is grain or a seed obtained or obtainable therefrom, wherein the grain or seed is characterized by increased expression of at least one nucleic acid and/or increased activity of a MRC polypeptide compared to a control or wild-type grain or seed.

7. A food or feed composition prepared from the grain or seed of claim 6.

8. A nucleic acid construct comprising a nucleic acid sequence encoding at least one DNA-binding domain or protospacer element that can bind to at least one target sequence in a MRC gene and/or promoter, wherein preferably the target sequence is selected from SEQ ID NO: 39 to 42 or a variant thereof, wherein preferably the sequence of the protospacer element is selected from SEQ ID Nos 43 to 46 or a variant thereof, wherein the variant has at least 60% overall sequence identity to one of SEQ ID NOs: 39 to 42 and 43 to 46.

9. A single guide (sg) RNA molecule wherein said sgRNA comprises a crRNA sequence and a tracrRNA sequence, wherein the crRNA sequence can bind to at least one sequence selected from SEQ ID Nos 39 to 42 or a variant thereof, and wherein preferably the sgRNA comprises or consists of a sequence selected from SEQ ID Nos 52 to 55 or a functional variant thereof, wherein the variant has at least 60% overall sequence identity to one of SEQ ID NOs 39 to 42 and 52 to 55.


SEQUENCE LISTING (108 sequences)

SEQ ID NO: 1 - protein, 773 aa, synthetic construct
  REGION 1..773: note = TaMRC 6A (cv. Kronos (4n))
MRLSIGSPSPSPPPAVAAALRSTPPSRRTASHVMFRQKLSFMEAFQTQHLKYAPRLIKSVVKGIRSNITDGDNGTTEPARELLERLFARTQSLDTGASHDSELSVSIEVLKSEFEGALSILRNKERDLRSAEKRVSDDRIRLSKTKQDLDQREEAIRKAYVRQQGIEKALKKASRDLALRVKQISDLKLLVEGQDRTIARSQALLSQKVTEVENLKRDMFKKNEEADLMRSEIRSKEKLLLTANQAIAQQEATVRELQSEIKRKTMDIARSNESRKTNEEKLKVAEQELEKQSLGWLAAQQELKELAQLAFKDTDDIKGIITDFKRVRSLLDAVRSELISSKDAFASSRRQIEDQAVQLQEQVQELEDQRVLLMSYTHDLEAAQLEIQGKTQELNYAQSRCHELESQLLKEMEKVESLEAELTKEKQSLEHRTEEVGFLQKELVQKENECTKSQELVKVKEFELLEARQEVQDMKLKVESIQLAVQEKDSELSDTQSRLTEVSSEIVELQQLLNSKKDQLVQARTELHDKEQHIETLESELDSIRLRCSQAESMVQRMAELTGDLASSVKAGEMDIYTLLDDEISSTSTALESNLHKHNQLEADIEMLRECLRHKDMELRAAHEALDAKDQELKAVLKKWDVKERELRELEELPDPSATNELAGFSSETTEGGIVGEMELPELQIDAAEVEALAATTALRKLADMTKDFFKHVKADSGINLVASESQKIIKCDPKMEVHKKTDVILEAEKEIVRLFSLTKQIVTDDIINDVEE

SEQ ID NO: 2 - protein, 773 aa, synthetic construct; identical to SEQ ID NO: 1
  REGION 1..773: note = TaMRC 6A (cv. Cadenza (6n))

SEQ ID NO: 3 - protein, 773 aa, synthetic construct
  REGION 1..773: note = TaMRC 6D (cv. Cadenza (6n))
MRLSIGSPSPSPPPAVAAALRSTSPSCRTASHVMFRQKLSFMVASQTQHLKYAPRLIKSVIKGIRSNITDGDNGTTEPARELLERLFAKTQSLDTGASHDSELSVSIEVLKSEFEGALSILRNKERDLRSAEKRVSDDRIRLSKTKQDLDQREEAIRKAYVRQQGIEKALKKASRDLALRVKQISNLKLQVEGQDRTIASSQALLSQKVIEVENLKRDMFKKNEEADLVRSEIRSKEQQLLTANKAIAQQEATVRELQSEIKRKTIDIARSNESRKTNEEKLKVAEQELEKQSLGWLAAQQELKELAQLAFKDTDDIKGIITDFKRVRSLLDAVRCELISSKDAFASSRRQIEDQAVQLQKQALELEDQQVLLMSYTHDLEAAQLEIQGKTQELKYAQSRCHELESQLLQEMEKVESLETELTKERQSLDHRTEEVGFLQKELVRKENECTKSQELVKVKEFELLEARQEVQDMKLKVESIQLAVQEKDSELSDTQSRLTEVSSEIVELQQLLNSKKDQLVQARTELHDKEQHIETLESELDSIRFRCSQAESMVQRMAELTGDLASSVKAGEMDIYTLLDDEISSTGTALESNLHKHNQLEADIEMLRECLRHKDMDLRAAHEALDAKDQELKAVLKKWDVKERELRELEELPDPSATNELAVFSSETTEDGIVGEMELPELQIEAAGVEALAATTALRKLADMTKDFFKHGKADSGIDLVASESQKISKCDPKMEVHKKTDVILEAEKEIVRLFSLTKQIVTDDTINNLEE

SEQ ID NO: 4 - DNA, 3073 bp, synthetic construct
  misc_feature 1..3073: note = 4 TaMRC 6A (cv. Kronos (4n))
  source 1..3073: mol_type = other DNA
cgatcgcgcccgggccgcggtggttccctctccccatgttccgcggccatgcgcctctccatcggctccccatccccgtcgccgccgccggcggtggccgccgctctccgcagcacacccccgtcgcgccgtaccgccagtcatgtgagcgcccgctgatcttttcttccttttctcatatcgctgtttcgtggtaccacgctgctcactgttacatggactgcttgcgttcgtgttttcccgattccgtgcccgtccacatgtgtttgaagtagaaggataccagattcggtgtcctaattcatgttctgctagtactagtactttttttaaaaacttttctggaattggttcaattgtgataaattcagtaaactgcgatcaaatttcatcagttatgatacttcaccttatgtagcagtgagtttctgaagttcagtgtactgtccttcagttcttccatttacaaaacaaatttttacgtgcttagtttgaggaaaggatattcctcagattgcttcactaggttgtgaccatttccttatcctaatatcctacttatgcattgtttcctgcaactctctcaggttatgttcaggcagaagctgagtttcatggaggcatttcagactcagcatctgaaatatgctcctcgtttgatcaaatcagtcgtaaaaggtattagatcaaatataactgatggtgataatggaacgactgagccagctagagagttgctggagcggctatttgcgaggacacaaagtttagacactggtgcttctcatgatagtgaactgagcgtgagcattgaggtcctgaagtctgaattcgagggtgccttgtctatcctcagaaacaaagagagggatcttcgcagcgcagagaagagggtttccgatgatcggataaggttgagcaagacaaagcaggaccttgatcagagagaggaagcgatccgcaaagcttatgtaaggcaacaaggaatagagaaagcactgaaaaaggcaagtagagatctggcgttgcgagtgaagcagatcagtgatctgaagcttctggttgaggggcaagacaggactattgccaggtcacaagctttgctttctcagaaggtaactgaagtggaaaatctcaaacgagatatgttcaagaagaacgaggaagcagacctgatgcgttcagagatcaggtccaaagaaaagctgcttcttacagctaatcaagctattgcgcagcaagaagcaacagttagggagctgcagagtgaaattaaaagaaagacaatggatatcgccagatcaaatgaatcgaggaaaactaatgaagagaaactgaaagttgctgaacaggaacttgagaagcagagtttaggatggttagcagcacaacaagagttaaaggaacttgcacaactggcattcaaagatacagatgatatcaagggtattatcactgacttcaaacgtgtgaggtctctgctagatgctgtacgctctgaattgatctcttcaaaagatgctttcgcttcctctcgcagacaaatagaagatcaagcggttcagttgcaggaacaagtacaggaactcgaggaccaaagggtattactgatgtcttacacccatgatttggaggctgctcaactggagattcaagggaagacacaggagctcaattatgcacagtctcgttgtcatgaacttgaatcacagttacttaaggaaatggagaaggtcgagtctctagaagccgaattaacgaaagaaaaacagagcttagaacatagaactgaagaagtaggctttcttcagaaggagcttgttcagaaagaaaatgagtgcaccaaatcacaagaacttgttaaagtaaaagagtttgagctgttagaagccagacaggaagtccaagatatgaagttaaaggtagagtctattcaattggctgttcaagaaaaggattcagagctttctgatacacagagcagactaactgaagtcagcagtgaaattgttgagcttcagcagttgctaaatagcaagaaggatcaactggttcaggctagaactgaattacatgataaagaacaacatatagaaacactggagagtgagttggatagcatacggctcagatgctcgcaagctgaatccatggttcaaaggatggctgagctcactggcgatcttgctagttccgtaaaagccggagaaatggacatctatacattactggatgatgaaatttcaagcacaagtacagccctcgagtccaatttacataagcataatcaactggaggctgacatagagatgttaagagaatgcttgcggcataaggacatggagttgagagctgctcatgaagcacttgatgccaaagatcaagagctgaaggcagtacttaaaaaatgggatgtgaaggagagggaactacgtgagttggaagagttaccggatcccagtgccacaaatgaacttgctggtttttccagtgagacaacagagggcggtattgtaggagagatggagctgccagagcttcaaattgatgctgcggaggtcgaagcacttgctgctacgactgcattgaggaagcttgcggatatgactaaggatttcttcaaacacgtcaaagctgattctggtattaacttggttgcatcagagagtcaaaaaatcattaaatgcgatcctaaaatggaagtacacaagaagacggatgtgattcttgaagctgaaaaagaaatagttaggctcttctcattgacaaaacagattgtcactgatgacataataaacgatgttgaggaatgatagcttcaaactaaagcatgtagtcttccaattctatcaaggtagatcttccaagatagcttcagagtagagatataccagatagatcgttcgaacatttatggacagcgatgtcgcccagaaggatgagatcttctctggttgatttcacaaactgccattttgaaaaagggtaacatgttgagcagaagctggtcatctgatcctttgtgctctttttgtaatgtgcctcaaactattcctcagatctttgttcaatg

SEQ ID NO: 5 - DNA, 3073 bp, synthetic construct
  misc_feature 1..3073: note = TaMRC 6A (cv. Cadenza (6n))
  source 1..3073: mol_type = other DNA
cgatcgcgcccgggccgcggtggttccctctccccatgttccgcggccatgcgcctctccatcggctccccatccccgtcgccgccgccggcggtggccgccgctctccgcagcacacccccgtcgcgccgtaccgccagtcatgtgagcgcccgctgatcttttcttccttttctcatatcgctgtttcgtggtaccacgctgctcactgttacatggactgcttgcgttcgtgttttcccgattccgtgcccgtccacatgtgtttgaagtagaaggataccagattcggtgtcctaattcatgttctgctagtactagtactttttttaaaaacttttctggaattggttcaattgtgataaattcagtaaactgcgatcaaatttcatcagttatgatacttcaccttatgtagcagtgagtttctgaagttcagtgtactgtccttcagttcttccatttacaaaacaaatttttacgtgcttagtttgaggaaaggatattcctcagattgcttcactaggttgtgaccatttccttatcctaatatcctacttatgcattgtttcctgcaactctctcaggttatgttcaggcagaagctgagtttcatggaggcatttcagactcagcatctgaaatatgctcctcgtttgatcaaatcagtcgtaaaaggtattagatcaaatataactgatggtgataatggaacgactgagccagctagagagttgctggagcggctatttgcgaggacacaaagtttagacactggtgcttctcatgatagtgaactgagcgtgagcattgaggtcctgaagtctgaattcgagggtgccttgtctatcctcagaaacaaagagagggatcttcgcagcgcagagaagagggtttccgatgatcggataaggttgagcaagacaaagcaggaccttgatcagagagaggaagcgatccgcaaagcttatgtaaggcaacaaggaatagagaaagcactgaaaaaggcaagtagagatctggcgttgcgagtgaagcagatcagtgatctgaagcttctggttgaggggcaagacaggactattgccaggtcacaagctttgctttctcagaaggtaactgaagtggaaaatctcaaacgagatatgttcaagaagaacgaggaagcagacctgatgcgttcagagatcaggtccaaagaaaagctgcttcttacagctaatcaagctattgcgcagcaagaagcaacagttagggagctgcagagtgaaattaaaagaaagacaatggatatcgccagatcaaatgaatcgaggaaaactaatgaagagaaactgaaagttgctgaacaggaacttgagaagcagagtttaggatggttagcagcacaacaagagttaaaggaacttgcacaactggcattcaaagatacagatgatatcaagggtattatcactgacttcaaacgtgtgaggtctctgctagatgctgtacgctctgaattgatctcttcaaaagatgctttcgcttcctctcgcagacaaatagaagatcaagcggttcagttgcaggaacaagtacaggaactcgaggaccaaagggtattactgatgtcttacacccatgatttggaggctgctcaactggagattcaagggaagacacaggagctcaattatgcacagtctcgttgtcatgaacttgaatcacagttacttaaggaaatggagaaggtcgagtctctagaagccgaattaacgaaagaaaaacagagcttagaacatagaactgaagaagtaggctttcttcagaaggagcttgttcagaaagaaaatgagtgcaccaaatcacaagaacttgttaaagtaaaagagtttgagctgttagaagccagacaggaagtccaagatatgaagttaaaggtagagtctattcaattggctgttcaagaaaaggactcagagctttctgatacacagagcagactaactgaagtcagcagtgaaattgttgagcttcagcagttgctaaatagcaagaaggatcaactggttcaggctagaactgaattacatgataaagaacaacatatagaaacactggagagtgagttggatagcatacggctcagatgctcgcaagctgaatccatggttcaaaggatggctgagctcactggcgatcttgctagttccgtaaaagccggagaaatggacatctatacattactggatgatgaaatttcaagcacaagtacagccctcgagtccaatttacataagcataatcaactggaggctgacatagagatgttaagagaatgcttgcggcataaggacatggagttgagagctgctcatgaagcacttgatgccaaagatcaagagctgaaggcagtacttaaaaaatgggatgtgaaggagagggaactacgtgagttggaagagttaccggatcccagtgccacaaatgaacttgctggtttttccagtgagacaacagagggcggtattgtaggagagatggagctgccagagcttcaaattgatgctgcggaggtcgaagcacttgctgctacgactgcattgaggaagcttgcggatatgactaaggatttcttcaaacacgtcaaagctgattctggtattaacttggttgcatcagagagtcaaaaaatcattaaatgcgatcctaaaatggaagtacacaagaagacggatgtgattcttgaagctgaaaaagaaatagttaggctcttctcattgacaaaacagattgtcactgatgacataataaacgatgttgaggaatgatagcttcaaactaaagcatgtagtcttccaattctatcaaggtagatcttccaagatagcttcagagtagagatataccagatagatcgttcgaacatttatggacagcgatgtcgcccagaaggatgagatcttctctggttgatttcacaaactgccattttgaaaaagggtaacatgttgagcagaagctggtcatctgatcctttgtgctctttttgtaatgtgcctcaaactattcctcagatctttgttcaatg

SEQ ID NO: 6 - DNA, 3162 bp, synthetic construct
  misc_feature 1..3162: note = TaMRC 6D (cv. Cadenza (6n))
  source 1..3162: mol_type = other DNA
cgatcgcgcccgggcggcggtggttccctctccccatgttccgcggccatgcgcctctccatcggctccccatccccgtcgccgccgccggcggtggccgccgctctccgcagcacatccccgtcgtgccgtaccgccagtcatgtgagcgcccgctgatcttttcttccttttctcatatcgctgtttcgtggtaccacgctgctcactgttacatgggctgcttgcgttcgtgttttcccgattccgtgcccgtccacatgtgtttgaagtagaaggatgccagatttggtgtcctaattcatgttctgctagtactagtaccttttttaaaacttttctggaattggttcgattgtgataaattcagtaaactgcacctggctgaacaaatcttgattggagaacggcctatgaactcaaaaaaaattactgaacagatgaaatgtttatgcagaggtaggcttgagatcaaatttcatcggttatgatacttcaccttatatagcagtgaatttctgaagttcagtgtactgtctttcagttcttcgatttacaaaacaaatttttacgtgcttagtttgaggaaaggatattcctcagattgcttcactaggttgtgaccatttccttatcctactatcctacttatgcattgtttcctgcaactctctcaggttatgttcaggcagaagctgagtttcatggtggcatctcagactcagcatctgaaatatgctcctcgtttgatcaaatcagtcataaaaggtattagatcaaatataactgatggtgataatggaacgactgagccagctagagagttgctggagcggctatttgcaaagacacaaagtttagacactggtgcttctcatgatagtgaactgagcgtgagcattgaggtcctgaagtctgaattcgagggtgccttgtctatcctcagaaacaaagagagggatcttcgcagcgcagagaagagggtttccgatgatcggataaggttgagcaagacaaagcaggaccttgatcagagagaggaagcgatccgcaaagcttatgtaaggcaacaaggaatagagaaagcactgaaaaaggcaagtagagatctggcgttgcgagtgaagcagatcagtaatctgaagcttcaggttgaggggcaagacaggactattgccagttcacaagctttgctttctcagaaggtaattgaggtggaaaatctcaaacgagatatgttcaagaagaacgaggaagccgacctggtgcgttcagagatcaggtccaaagagcagcagcttcttacagctaataaagctattgcgcagcaagaagcaacagttagggagctgcagagtgaaattaaaagaaagacaatcgatatcgccagatcaaatgaatcgaggaaaactaatgaagagaaactgaaagttgctgaacaggaacttgagaagcagagtttaggatggttagcagcacaacaagagttaaaggaacttgcacaactggcattcaaagatacagatgatatcaagggtattatcactgacttcaaacgtgtgaggtctctcctagatgctgtacgctgtgaattaatctcttcgaaagatgctttcgcttcctctcgcagacaaatagaagatcaagcggtgcagttgcagaaacaagcactggaactcgaggaccaacaggtattactgatgtcttacacccatgatttggaagctgctcaactggagattcaagggaagacacaggagctcaagtacgcacagtctcgttgtcatgaacttgaatcacagttacttcaggaaatggagaaggtcgagtctcttgaaaccgaattaaccaaagaaagacagagcttagatcatagaactgaagaagtaggctttcttcagaaggagcttgttcggaaagaaaatgagtgcaccaaatcacaagaacttgttaaagtaaaagagtttgagctgttagaagccagacaggaagtccaagatatgaagttaaaggtagagtctattcaattggctgttcaagaaaaggattcagagctttctgatacacagagcagactaactgaagtcagcagtgaaattgttgagcttcagcagttgctaaatagcaagaaggatcaactggttcaggctagaactgaattacatgataaagaacaacatatagaaacactggagagtgagttggatagcatacggttcagatgctcgcaagctgaatccatggttcaaaggatggctgagctcactggcgatcttgctagttccgtaaaagctggagaaatggacatctatacattactggatgatgaaatttcaagcacaggtacagccctcgagtccaatttgcataagcataatcaactggaggctgacatagagatgttaagagaatgcttgcggcataaggacatggacttgagagctgctcatgaagcacttgatgccaaagatcaagagctgaaggcagtacttaaaaagtgggatgtgaaggagagggaactacgtgagttggaagagttaccggatcccagtgccacaaatgaacttgctgttttttccagtgagacaacagaggacggcattgtaggagagatggagctccctgagcttcaaattgaagctgcgggggtcgaagcacttgctgctacgactgcattgaggaagcttgcggatatgactaaggatttcttcaaacacggcaaagctgattctggtattgacttggttgcatcagagagtcaaaaaatcagtaaatgtgatcctaaaatggaagtacacaagaagacggatgtgattcttgaagctgaaaaagaaatagttaggctcttctcattgacaaaacagattgtcactgatgacacaataaacaatcttgaggaatgatagcttcaaactaaagcatgtagtcttccaattctatcaaggtagatcttccaagatagcttcagagtagagatataccagatagatctttcaaacattgatggacagcgacgtcgcccagaaggatgagatcttctctggttgatatcacaactgccattttgaaaaagggtaacatgttgagcagaagctggtcatctgatcttttgtgctccttttgtattgtacctcaagctattcctcagatctttgttcaatg

SEQ ID NO: 7 - DNA, 2322 bp, synthetic construct
  misc_feature 1..2322: note = TaMRC 6A (cv. Kronos (4n)) CDS
  source 1..2322: mol_type = other DNA
atgcgcctctccatcggctccccatccccgtcgccgccgccggcggtggccgccgctctccgcagcacacccccgtcgcgccgtaccgccagtcatgttatgttcaggcagaagctgagtttcatggaggcatttcagactcagcatctgaaatatgctcctcgtttgatcaaatcagtcgtaaaaggtattagatcaaatataactgatggtgataatggaacgactgagccagctagagagttgctggagcggctatttgcgaggacacaaagtttagacactggtgcttctcatgatagtgaactgagcgtgagcattgaggtcctgaagtctgaattcgagggtgccttgtctatcctcagaaacaaagagagggatcttcgcagcgcagagaagagggtttccgatgatcggataaggttgagcaagacaaagcaggaccttgatcagagagaggaagcgatccgcaaagcttatgtaaggcaacaaggaatagagaaagcactgaaaaaggcaagtagagatctggcgttgcgagtgaagcagatcagtgatctgaagcttctggttgaggggcaagacaggactattgccaggtcacaagctttgctttctcagaaggtaactgaagtggaaaatctcaaacgagatatgttcaagaagaacgaggaagcagacctgatgcgttcagagatcaggtccaaagaaaagctgcttcttacagctaatcaagctattgcgcagcaagaagcaacagttagggagctgcagagtgaaattaaaagaaagacaatggatatcgccagatcaaatgaatcgaggaaaactaatgaagagaaactgaaagttgctgaacaggaacttgagaagcagagtttaggatggttagcagcacaacaagagttaaaggaacttgcacaactggcattcaaagatacagatgatatcaagggtattatcactgacttcaaacgtgtgaggtctctgctagatgctgtacgctctgaattgatctcttcaaaagatgctttcgcttcctctcgcagacaaatagaagatcaagcggttcagttgcaggaacaagtacaggaactcgaggaccaaagggtattactgatgtcttacacccatgatttggaggctgctcaactggagattcaagggaagacacaggagctcaattatgcacagtctcgttgtcatgaacttgaatcacagttacttaaggaaatggagaaggtcgagtctctagaagccgaattaacgaaagaaaaacagagcttagaacatagaactgaagaagtaggctttcttcagaaggagcttgttcagaaagaaaatgagtgcaccaaatcacaagaacttgttaaagtaaaagagtttgagctgttagaagccagacaggaagtccaagatatgaagttaaaggtagagtctattcaattggctgttcaagaaaaggattcagagctttctgatacacagagcagactaactgaagtcagcagtgaaattgttgagcttcagcagttgctaaatagcaagaaggatcaactggttcaggctagaactgaattacatgataaagaacaacatatagaaacactggagagtgagttggatagcatacggctcagatgctcgcaagctgaatccatggttcaaaggatggctgagctcactggcgatcttgctagttccgtaaaagccggagaaatggacatctatacattactggatgatgaaatttcaagcacaagtacagccctcgagtccaatttacataagcataatcaactggaggctgacatagagatgttaagagaatgcttgcggcataaggacatggagttgagagctgctcatgaagcacttgatgccaaagatcaagagctgaaggcagtacttaaaaaatgggatgtgaaggagagggaactacgtgagttggaagagttaccggatcccagtgccacaaatgaacttgctggtttttccagtgagacaacagagggcggtattgtaggagagatggagctgccagagcttcaaattgatgctgcggaggtcgaagcacttgctgctacgactgcattgaggaagcttgcggatatgactaaggatttcttcaaacacgtcaaagctgattctggtattaacttggttgcatcagagagtcaaaaaatcattaaatgcgatcctaaaatggaagtacacaagaagacggatgtgattcttgaagctgaaaaagaaatagttaggctcttctcattgacaaaacagattgtcactgatgacataataaacgatgttgaggaatga

SEQ ID NO: 8 - DNA, 2322 bp, synthetic construct
  misc_feature 1..2322: note = TaMRC 6A (cv. Cadenza (6n)) CDS
  source 1..2322: mol_type = other DNA
atgcgcctctccatcggctccccatccccgtcgccgccgccggcggtggccgccgctctccgcagcacacccccgtcgcgccgtaccgccagtcatgttatgttcaggcagaagctgagtttcatggaggcatttcagactcagcatctgaaatatgctcctcgtttgatcaaatcagtcgtaaaaggtattagatcaaatataactgatggtgataatggaacgactgagccagctagagagttgctggagcggctatttgcgaggacacaaagtttagacactggtgcttctcatgatagtgaactgagcgtgagcattgaggtcctgaagtctgaattcgagggtgccttgtctatcctcagaaacaaagagagggatcttcgcagcgcagagaagagggtttccgatgatcggataaggttgagcaagacaaagcaggaccttgatcagagagaggaagcgatccgcaaagcttatgtaaggcaacaaggaatagagaaagcactgaaaaaggcaagtagagatctggcgttgcgagtgaagcagatcagtgatctgaagcttctggttgaggggcaagacaggactattgccaggtcacaagctttgctttctcagaaggtaactgaagtggaaaatctcaaacgagatatgttcaagaagaacgaggaagcagacctgatgcgttcagagatcaggtccaaagaaaagctgcttcttacagctaatcaagctattgcgcagcaagaagcaacagttagggagctgcagagtgaaattaaaagaaagacaatggatatcgccagatcaaatgaatcgaggaaaactaatgaagagaaactgaaagttgctgaacaggaacttgagaagcagagtttaggatggttagcagcacaacaagagttaaaggaacttgcacaactggcattcaaagatacagatgatatcaagggtattatcactgacttcaaacgtgtgaggtctctgctagatgctgtacgctctgaattgatctcttcaaaagatgctttcgcttcctctcgcagacaaatagaagatcaagcggttcagttgcaggaacaagtacaggaactcgaggaccaaagggtattactgatgtcttacacccatgatttggaggctgctcaactggagattcaagggaagacacaggagctcaattatgcacagtctcgttgtcatgaacttgaatcacagttacttaaggaaatggagaaggtcgagtctctagaagccgaattaacgaaagaaaaacagagcttagaacatagaactgaagaagtaggctttcttcagaaggagcttgttcagaaagaaaatgagtgcaccaaatcacaagaacttgttaaagtaaaagagtttgagctgttagaagccagacaggaagtccaagatatgaagttaaaggtagagtctattcaattggctgttcaagaaaaggactcagagctttctgatacacagagcagactaactgaagtcagcagtgaaattgttgagcttcagcagttgctaaatagcaagaaggatcaactggttcaggctagaactgaattacatgataaagaacaacatatagaaacactggagagtgagttggatagcatacggctcagatgctcgcaagctgaatccatggttcaaaggatggctgagctcactggcgatcttgctagttccgtaaaagccggagaaatggacatctatacattactggatgatgaaatttcaagcacaagtacagccctcgagtccaatttacataagcataatcaactggaggctgacatagagatgttaagagaatgcttgcggcataaggacatggagttgagagctgctcatgaagcacttgatgccaaagatcaagagctgaaggcagtacttaaaaaatgggatgtgaaggagagggaactacgtgagttggaagagttaccggatcccagtgccacaaatgaacttgctggtttttccagtgagacaacagagggcggtattgtaggagagatggagctgccagagcttcaaattgatgctgcggaggtcgaagcacttgctgctacgactgcattgaggaagcttgcggatatgactaaggatttcttcaaacacgtcaaagctgattctggtattaacttggttgcatcagagagtcaaaaaatcattaaatgcgatcctaaaatggaagtacacaagaagacggatgtgattcttgaagctgaaaaagaaatagttaggctcttctcattgacaaaacagattgtcactgatgacataataaacgatgttgaggaatga

SEQ ID NO: 9 - DNA, 2322 bp, synthetic construct
  misc_feature 1..2322: note = TaMRC 6D (cv. Cadenza (6n)) CDS
  source 1..2322: mol_type = other DNA
atgcgcctctccatcggctccccatccccgtcgccgccgccggcggtggccgccgctctccgcagcacatccccgtcgtgccgtaccgccagtcatgttatgttcaggcagaagctgagtttcatggtggcatctcagactcagcatctgaaatatgctcctcgtttgatcaaatcagtcataaaaggtattagatcaaatataactgatggtgataatggaacgactgagccagctagagagttgctggagcggctatttgcaaagacacaaagtttagacactggtgcttctcatgatagtgaactgagcgtgagcattgaggtcctgaagtctgaattcgagggtgccttgtctatcctcagaaacaaagagagggatcttcgcagcgcagagaagagggtttccgatgatcggataaggttgagcaagacaaagcaggaccttgatcagagagaggaagcgatccgcaaagcttatgtaaggcaacaaggaatagagaaagcactgaaaaaggcaagtagagatctggcgttgcgagtgaagcagatcagtaatctgaagcttcaggttgaggggcaagacaggactattgccagttcacaagctttgctttctcagaaggtaattgaggtggaaaatctcaaacgagatatgttcaagaagaacgaggaagccgacctggtgcgttcagagatcaggtccaaagagcagcagcttcttacagctaataaagctattgcgcagcaagaagcaacagttagggagctgcagagtgaaattaaaagaaagacaatcgatatcgccagatcaaatgaatcgaggaaaactaatgaagagaaactgaaagttgctgaacaggaacttgagaagcagagtttaggatggttagcagcacaacaagagttaaaggaacttgcacaactggcattcaaagatacagatgatatcaagggtattatcactgacttcaaacgtgtgaggtctctcctagatgctgtacgctgtgaattaatctcttcgaaagatgctttcgcttcctctcgcagacaaatagaagatcaagcggtgcagttgcagaaacaagcactggaactcgaggaccaacaggtattactgatgtcttacacccatgatttggaagctgctcaactggagattcaagggaagacacaggagctcaagtacgcacagtctcgttgtcatgaacttgaatcacagttacttcaggaaatggagaaggtcgagtctcttgaaaccgaattaaccaaagaaagacagagcttagatcatagaactgaagaagtaggctttcttcagaaggagcttgttcggaaagaaaatgagtgcaccaaatcacaagaacttgttaaagtaaaagagtttgagctgttagaagccagacaggaagtccaagatatgaagttaaaggtagagtctattcaattggctgttcaagaaaaggattcagagctttctgatacacagagcagactaactgaagtcagcagtgaaattgttgagcttcagcagttgctaaatagcaagaaggatcaactggttcaggctagaactgaattacatgataaagaacaacatatagaaacactggagagtgagttggatagcatacggttcagatgctcgcaagctgaatccatggttcaaaggatggctgagctcactggcgatcttgctagttccgtaaaagctggagaaatggacatctatacattactggatgatgaaatttcaagcacaggtacagccctcgagtccaatttgcataagcataatcaactggaggctgacatagagatgttaagagaatgcttgcggcataaggacatggacttgagagctgctcatgaagcacttgatgccaaagatcaagagctgaaggcagtacttaaaaagtgggatgtgaaggagagggaactacgtgagttggaagagttaccggatcccagtgccacaaatgaacttgctgttttttccagtgagacaacagaggacggcattgtaggagagatggagctccctgagcttcaaattgaagctgcgggggtcgaagcacttgctgctacgactgcattgaggaagcttgcggatatgactaaggatttcttcaaacacggcaaagctgattctggtattgacttggttgcatcagagagtcaaaaaatcagtaaatgtgatcctaaaatggaagtacacaagaagacggatgtgattcttgaagctgaaaaagaaatagttaggctcttctcattgacaaaacagattgtcactgatgacacaataaacaatcttgaggaatga

SEQ ID NO: 10 - protein, 774 aa, synthetic construct
  REGION 1..774: note = HvMRC (HORVU6Hr1G036020.1)
MRLSTGCPSPSPAAALAAAHRSTSPSCRTATHVMFRHKLSFMVAFQTQHLKYAPCLIKSVVKSIRSNITDGDNGTTEPARELLERLFAKTQSLDTGASNDSELGVSIEVLKSEFEGALSILRKKERDLRNAEKRVSDDRTRLSKTKQDLDQREETIRKVYVRQQDIEKALKRASRDLALRVKQISNLKLLVEGQDRTIASSQALLSQKVIEVENLKQDMFTKNEEADLMRSEIKSKEQLLLTANQAVVQQEATVRELQSEIKRKIIDIARSDELRKTNEDKLKVAEQELEKQNLGWLAAQQELKELAQLASDDTDDIKGIITDFKRVRSLLDVVRSELISSKDAFASSRRQIEDQAVQLREQVQELEDQRVLLMSHTHDLEAARLEIQGKTQELNYAQSRCHELESHLLQEMEKVESLEAELTKERQSLEHRTEEVDFLQKELVQKENECTKSQELVKVKEFELLEARYEVQDMKLKVESIQLAVQEKDSELSATQSRLTEVSSEVVKLQQLLNSKEDQLVQARTELHDKEQHIETLESELDSIRLRCSQAESVVQRMAELTGDLASSVKTGETDIYTLLDDEIASAGTTLESNLHKHNQLEADIEMLRECLRHKDMDLRAAHEALDAKDQELKAVLKKWDVKERELHELEELLDPSATNELACFSNETTEGGVVGEMELQELQIGAAEVEALAATTALRKLADMTKDLFKHDKGDSGIDLAASGSQKLRNCDSKMEVHKKTDVILEAEKEITRLFSLTKQIVTDDIINDVDER

SEQ ID NO: 11 - DNA, 3217 bp, synthetic construct
  misc_feature 1..3217: note = HvMRC (HORVU6Hr1G036020 cv. Morex)
  source 1..3217: mol_type = other DNA
cgatcgcgcccgggcggcggtggttccctctccccatgttccgcggccatgcgcctctccaccggctgcccatccccctcgccggcggcggcgctggccgccgctcaccgcagcacatccccgtcgtgccgtaccgccactcatgtgagcacccgctgatcttttcttccttctcccatatcactgtttcctggtaccacgctgctcactgttacatgggctgcttgtgttcacatgtgtttgaagtagagatttggcgtcgctaattcatgttctgccagtatcggtactttttttacttttctggaattggttctattgtgataaattctgtaaactgtacctggctgaacaaatcttgattggagaactgcctataaactcaaaaattgtaccgagcagatgaaatatgtatgcaggggcaggtttgaggtcaaatttcatcagttatgatacctcaccttatattaatagcagtgaatttgtctgaagagttgtacctgattttttctttctggagttcagtgccctgccctttcagttcttcgatttacaaaacaatttttacgtccttagtttgaggaaagaatattcctcggataatagcttcactattgttctcgaaaaaagataatagcttcactagcttgtgccctgcttcacttatcctaatatgctacttatgcattgtttcctgcaactctctcaggttatgttcagacataagctgagtttcatggtggcatttcagactcagcatctgaaatatgctccttgcttgatcaaatcagtcgtaaaaagtattagatcaaatataactgatggtgataatggaacgactgagccagctagagaattgctggagcggctatttgcgaagacacaaagtttagacactggtgcttcaaatgacagtgaactgggcgtgagcattgaggtcctgaaatctgaattcgagggtgccttgtctatcctcagaaagaaagagagggatcttcgcaacgcagagaagagggtttccgatgatcggacaaggttgagcaagacgaagcaggaccttgatcagagagaggagacgatccgcaaagtttatgtaaggcaacaagatatagagaaagcactgaaaagggcaagtagagatctggcgttgcgagtgaagcagatcagtaatctgaagcttctggttgaggggcaagataggactattgccagttcacaagctttgctttctcagaaggtaattgaagtggaaaatctcaaacaagacatgttcacaaagaacgaggaagctgacctgatgcgttcagagatcaagtccaaagaacagctgcttcttacagctaatcaagctgttgtgcagcaagaagcaacagttagggagctgcagagtgaaattaaaagaaagataatcgatatcgccagatcagatgaattgaggaaaactaatgaagataaactgaaagttgctgaacaggaacttgagaagcagaatttaggatggttagcagcacagcaagagttaaaggaacttgcccaactggcatccgatgatacagatgatatcaagggtattatcactgacttcaaacgtgtgaggtctctgctagatgttgtacgctctgaattgatctcttcaaaagatgctttcgcttcctctcgcagacaaatagaagatcaagcggtgcagctgcgggaacaagtacaggaacttgaggaccaaagggtattgctgatgtctcacacccatgatttggaggctgctcgactggagattcaagggaagacacaggagctcaattacgcacagtctcgttgtcatgaacttgagtcacatttacttcaggaaatggagaaggtcgagtctctagaagccgaattaaccaaagaaagacagagcttagaacatagaactgaagaagtagactttcttcagaaggagcttgtacagaaagaaaatgagtgcaccaaatcacaagaacttgttaaagtaaaagagtttgagctgttagaagccagatatgaagtccaagatatgaagttaaaggtagagtctattcaattggctgttcaagaaaaggattcagagctttctgctacacagagcagactaactgaagtcagcagtgaagttgttaaacttcagcagttgctaaatagcaaggaggatcaactggttcaggctagaactgaattgcatgataaagaacaacatatagaaacactggagagtgaattggatagcatacgactcagatgctcgcaagctgaatccgtggttcaaaggatggctgagctcactggcgatcttgctagttccgtaaaaactggagaaacggacatctatacattactggatgatgaaattgcaagcgcaggtacaaccctcgagtccaatttgcataagcataatcaactggaggctgacatagagatgttaagagaatgcttgcggcataaggacatggacttgagagctgctcatgaagcacttgatgccaaagatcaagagctgaaggcagtacttaaaaagtgggatgtgaaggagagggaactacatgagttggaagagttactggatcccagtgccacaaatgaacttgcttgtttctccaatgagacaaccgagggcggagttgtaggagagatggagctccaagagcttcaaattggagctgcggaggtggaagcacttgctgctacgactgcattgaggaagcttgcagacatgactaaggatctcttcaaacacgacaaaggtgattctggtattgatttggctgcatcagggagtcaaaaactcagaaattgtgattctaaaatggaagtacacaagaagacggatgtgattcttgaagctgaaaaagaaataactaggctcttctcattgacaaaacagattgttactgatgacataataaacgatgttgatgaacgatagcttcaaactaaagcatgtagtcttccaattctatcgaaggtagatcttccaagatagcttcagagtagtaatataccagatagatctttccaacattatggacagtgacgttgcccagaaagataagatcttctctagttgatttgacaactgccattttgaaaaagggtaacttatttagcagaagctggtcatttgatcctttgtcccctttttgtaatgtacccaaactattccttgtatctttgttcaattatgttccctctaaatatacgtggg

SEQ ID NO: 12 - DNA, 2325 bp, synthetic construct
  misc_feature 1..2325: note = HvMRC CDS (HORVU6Hr1G036020.1 cv. Morex)
  source 1..2325: mol_type = other DNA
atgcgcctctccaccggctgcccatccccctcgccggcggcggcgctggccgccgctcaccgcagcacatccccgtcgtgccgtaccgccactcatgttatgttcagacataagctgagtttcatggtggcatttcagactcagcatctgaaatatgctccttgcttgatcaaatcagtcgtaaaaagtattagatcaaatataactgatggtgataatggaacgactgagccagctagagaattgctggagcggctatttgcgaagacacaaagtttagacactggtgcttcaaatgacagtgaactgggcgtgagcattgaggtcctgaaatctgaattcgagggtgccttgtctatcctcagaaagaaagagagggatcttcgcaacgcagagaagagggtttccgatgatcggacaaggttgagcaagacgaagcaggaccttgatcagagagaggagacgatccgcaaagtttatgtaaggcaacaagatatagagaaagcactgaaaagggcaagtagagatctggcgttgcgagtgaagcagatcagtaatctgaagcttctggttgaggggcaagataggactattgccagttcacaagctttgctttctcagaaggtaattgaagtggaaaatctcaaacaagacatgttcacaaagaacgaggaagctgacctgatgcgttcagagatcaagtccaaagaacagctgcttcttacagctaatcaagctgttgtgcagcaagaagcaacagttagggagctgcagagtgaaattaaaagaaagataatcgatatcgccagatcagatgaattgaggaaaactaatgaagataaactgaaagttgctgaacaggaacttgagaagcagaatttaggatggttagcagcacagcaagagttaaaggaacttgcccaactggcatccgatgatacagatgatatcaagggtattatcactgacttcaaacgtgtgaggtctctgctagatgttgtacgctctgaattgatctcttcaaaagatgctttcgcttcctctcgcagacaaatagaagatcaagcggtgcagctgcgggaacaagtacaggaacttgaggaccaaagggtattgctgatgtctcacacccatgatttggaggctgctcgactggagattcaagggaagacacaggagctcaattacgcacagtctcgttgtcatgaacttgagtcacatttacttcaggaaatggagaaggtcgagtctctagaagccgaattaaccaaagaaagacagagcttagaacatagaactgaagaagtagactttcttcagaaggagcttgtacagaaagaaaatgagtgcaccaaatcacaagaacttgttaaagtaaaagagtttgagctgttagaagccagatatgaagtccaagatatgaagttaaaggtagagtctattcaattggctgttcaagaaaaggattcagagctttctgctacacagagcagactaactgaagtcagcagtgaagttgttaaacttcagcagttgctaaatagcaaggaggatcaactggttcaggctagaactgaattgcatgataaagaacaacatatagaaacactggagagtgaattggatagcatacgactcagatgctcgcaagctgaatccgtggttcaaaggatggctgagctcactggcgatcttgctagttccgtaaaaactggagaaacggacatctatacattactggatgatgaaattgcaagcgcaggtacaaccctcgagtccaatttgcataagcataatcaactggaggctgacatagagatgttaagagaatgcttgcggcataaggacatggacttgagagctgctcatgaagcacttgatgccaaagatcaagagctgaaggcagtacttaaaaagtgggatgtgaaggagagggaactacatgagttggaagagttactggatcccagtgccacaaatgaacttgcttgtttctccaatgagacaaccgagggcggagttgtaggagagatggagctccaagagcttcaaattggagctgcggaggtggaagcacttgctgctacgactgcattgaggaagcttgcagacatgactaaggatctcttcaaacacgacaaaggtgattctggtattgatttggctgcatcagggagtcaaaaactcagaaattgtgattctaaaatggaagtacacaagaagacggatgtgattcttgaagctgaaaaagaaataactaggctcttctcattgacaaaacagattgttactgatgacataataaacgatgttgatgaacgatag

SEQ ID NO: 13 - protein, 780 aa, synthetic construct
  REGION 1..780: note = Brachypodium (Bradi3g06260)
MFRGHAPLHRLPSPPPPPAAAAGALPSASPSCRTSTHVPFRPKLSFMVAFQAQHVKYAPNLIKSVVKSLRSNITDGDNGMTEPARELLERLFAKTQSLDTSASHDSELSMSIEVLKSEFERALSILRKKERYLRNAEKRVSDDQLRLNQTKQDLDQREQEISKAHAKQQQMEKALKKASRDLSLRVKQINNLKLLVERQDRKIASSEALLSQKVIEVENLKQDMFNKNKEADLIRSEIKLKEQLLLEANQDVVQQEATVRELRSETEKKAIDIAISNELRKANEEKLKIAEQELEKQNLGWLAAQQELKELAQLASKDTDDIKGTVTDFKRVRSLLDAVRSELISSKDNFASSRRQIEEQTVQLQKQVQELKDQRVLLMSYTQDLEAAQLEIQGKTKDLNAAQSRCHELELQLLKEMEKVESLEAELTKERENLEQKTEQVDFLQKELVQKENECGNSQKLVKIKEAELLEARHEVQDMKSKVDSIQLAVQEKDSELSDTQSRLTEVSGEVVELQQLLNSKDDQLVQVRTELHDKEQYIESMQSELESIRFRCSQAESVLRRMAELTGDLASSVKAGEMDIYALLDDEISSTSTVLESNLHKHNQLEADIEMLRESLRHKDMDLRAAHEALDAKDQELKAVVGKWDFKEKELDEVEELQKDPIDMKELPVLSNETTGGSITGEMELKKLQIEAAEVEALAATTALKKLADMSKKYLRCRKADSGIGLVASESANIGKANSRMELNNKMDVIFEAKQEIVRLFSLTKELITDDAINDAEER

SEQ ID NO: 14 - DNA, 3615 bp, synthetic construct
  misc_feature 1..3615: note = Brachypodium (Bradi3g06260)
  source 1..3615: mol_type = other DNA
ctcgtgccgtctcggcgtcttcctccacagtccacaaacccgcacgcgcatcgccagccgtccgatcgcgctcgggcggccctggtccgcttcctcaaatgttccgcggccatgcccctctccaccggctcccctcgccgccgccgccaccggcggcggccgccggcgcactccccagcgcgtccccgtcgtgccgcacctccactcatgtgagcaattgctgttttttctgtatcttcctcatatgtctgtttctgatttggctctaccacgctgcctgcgctattggaatccttcttcgttcccccgattccgagccccgcctgcgtgcgtttgaagtggaaggggaccggattttgcgtttgctaattcatggtctgttagtagttccttagttttctggagttagttcgattttgataaatccaatataatagcttctggtagaacaaatcctgattacagaaaggcctaatgggctatcaactaaaaatggtatagataacaaaagttgagcttccctagcctgtgcttatttccttatcccaatagtctaagcattgttttctgcaaccctatcaggttcccttcaggccgaagttgagcttcatggtggcatttcaggctcaacatgtgaaatatgctcctaacctgatcaaatcagtagtaaaaagtcttagatcaaacatcactgatggtgacaatggaatgaccgagccagctagggaattgttggaacggctgtttgcgaagacacagagtctagacacaagtgcttctcatgatagtgaactgagcatgagcatcgaggtcctcaagtctgaattcgagcgcgccttgtcgattctcagaaagaaagagaggtaccttcggaatgcagagaagagggtttctgatgatcagttaaggttgaaccagacgaagcaggacctggatcagagagagcaagagatcagcaaagcacatgcaaagcagcaacaaatggagaaagcactgaaaaaggcaagtagagatctgtcgttgcgagtgaagcagatcaataatctgaagcttctggttgagaggcaagacaggaaaattgccagttcagaagctttgctttctcaaaaggtaattgaagtggaaaatctcaaacaagatatgttcaacaagaataaggaagcagacttgataagatcagagattaagttgaaagaacaactgcttcttgaagctaatcaggacgtcgtgcagcaagaggcaacagttagggagctgcggagtgaaactgaaaaaaaggctattgatattgccatatccaatgaattgaggaaggctaatgaagagaaactgaaaattgctgaacaggaacttgagaagcagaatttaggatggttagcagcacagcaagaattaaaggaactggcgcaacttgcatccaaggacacagatgatatcaagggtactgtcactgactttaaacgtgtgaggtccctgctggatgctgtacggtctgaactaatctcttcaaaagataatttcgcctcctctcgcagacaaatagaagaacaaacggtgcagttgcagaagcaagtgcaagaactcaaggaccaaagggtattgctgatgtcttacacccaggatttggaagctgctcaactggagattcaagggaagacaaaagatctcaatgctgcacagtctcgttgccatgaacttgaattgcagttacttaaggaaatggagaaggttgagtctctagaagccgagttaaccaaagaaagagagaacttggaacagaaaactgaacaagtagactttcttcagaaggagcttgttcagaaggaaaatgagtgtggtaattcacaaaagcttgttaaaataaaagaggcagagctattagaagccagacatgaagtccaagatatgaaatcaaaggtagattctatccaattggctgttcaagagaaggattcagagctttcggacacacagagcagactaactgaagtgagcggtgaagttgttgagcttcagcagttgctaaatagcaaggatgatcaacttgttcaggttagaactgagttacatgataaagaacaatatatagaatcaatgcagagtgaattagagagcataagattcagatgctcgcaagctgaatctgtgttgcgaaggatggctgagctcactggcgatcttgctagttccgtgaaagctggagaaatggacatttatgcattactggatgatgaaatttcaagcaccagtacagtccttgagtccaatttgcacaagcataatcaactggaggctgacatagagatgttaagagaatccttacggcataaggacatggacttaagagctgctcatgaagcacttgatgccaaagatcaagaactgaaggcagtagttggaaagtgggatttcaaggagaaggaactggatgaggtggaagagttacagaaagatcccattgacatgaaggaactccctgttctttctaacgagacaacagggggcagcattacaggagagatggagctcaagaagcttcaaattgaagctgccgaggtggaggcacttgctgctactactgcactaaagaagcttgcggatatgagtaagaaatacttgagatgccgcaaagctgattctgggattggtttggttgcatcagaaagtgcaaacattggtaaagcgaattctaggatggaattaaacaacaagatggatgtgatttttgaagctaaacaagaaattgttagactattttcattgacaaaagagctcatcactgatgacgcaataaatgatgctgaggaacgataactttagagctaaaatattcagccagccaattctaccaagatagcttcagaatagaggtatggcagatagatctcagacatttatgagcagctgggtcgcatagcaagaccaaaatctgtcgctggttgattcggcaaatggcgttctaacaaaggataaaagaaactgtccatgtgtgtattttcggagacgaacctcaaactcttcctaataatgttttccgatgtgttgctgtaaatatatgtggggagttactggtatggcatttggtgctggttgtccccacacactgtacattggacgatgctccaggttttgttgttggttaactgaaatgatgttcagtgtttgctattgttgttgcaccacaaagtggatgatgttccaaattagagcaacaaggaaagaaagggagatgactgtcaagaaaaagggaaagaaaggagatagctatagttcatctaaaaaggggagatggaaattttgagctggaagtcaccaatcttcaaggcgcaaccttttaaattttgcacagaaagtattcagtatttcagggtttaactgttaggattctgttatttgtagagtctttaagattctattccaagtgcaatgcccctcaatgtcacttcgaggcagagacaaactctatctctatgctttctcagtttctcttaactccaggtcaccaaaattcttatgtggcaatacccttacaggtagttgatgtaagcctatgcatacattgcatagtcagttctttctctgattgagaggcactaactttcttcatcagataaa

SEQ ID NO: 15 - DNA, 2343 bp, synthetic construct
  misc_feature 1..2343: note = Brachypodium CDS (Bradi3g06260)
  source 1..2343: mol_type = other DNA
atgttccgcggccatgcccctctccaccggctcccctcgccgccgccgccaccggcggcggccgccggcgcactccccagcgcgtccccgtcgtgccgcacctccactcatgttcccttcaggccgaagttgagcttcatggtggcatttcaggctcaacatgtgaaatatgctcctaacctgatcaaatcagtagtaaaaagtcttagatcaaacatcactgatggtgacaatggaatgaccgagccagctagggaattgttggaacggctgtttgcgaagacacagagtctagacacaagtgcttctcatgatagtgaactgagcatgagcatcgaggtcctcaagtctgaattcgagcgcgccttgtcgattctcagaaagaaagagaggtaccttcggaatgcagagaagagggtttctgatgatcagttaaggttgaaccagacgaagcaggacctggatcagagagagcaagagatcagcaaagcacatgcaaagcagcaacaaatggagaaagcactgaaaaaggcaagtagagatctgtcgttgcgagtgaagcagatcaataatctgaagcttctggttgagaggcaagacaggaaaattgccagttcagaagctttgctttctcaaaaggtaattgaagtggaaaatctcaaacaagatatgttcaacaagaataaggaagcagacttgataagatcagagattaagttgaaagaacaactgcttcttgaagctaatcaggacgtcgtgcagcaagaggcaacagttagggagctgcggagtgaaactgaaaaaaaggctattgatattgccatatccaatgaattgaggaaggctaatgaagagaaactgaaaattgctgaacaggaacttgagaagcagaatttaggatggttagcagcacagcaagaattaaaggaactggcgcaacttgcatccaaggacacagatgatatcaagggtactgtcactgactttaaacgtgtgaggtccctgctggatgctgtacggtctgaactaatctcttcaaaagataatttcgcctcctctcgcagacaaatagaagaacaaacggtgcagttgcagaagcaagtgcaagaactcaaggaccaaagggtattgctgatgtcttacacccaggatttggaagctgctcaactggagattcaagggaagacaaaagatctcaatgctgcacagtctcgttgccatgaacttgaattgcagttacttaaggaaatggagaaggttgagtctctagaagccgagttaaccaaagaaagagagaacttggaacagaaaactgaacaagtagactttcttcagaaggagcttgttcagaaggaaaatgagtgtggtaattcacaaaagcttgttaaaataaaagaggcagagctattagaagccagacatgaagtccaagatatgaaatcaaaggtagattctatccaattggctgttcaagagaaggattcagagctttcggacacacagagcagactaactgaagtgagcggtgaagttgttgagcttcagcagttgctaaatagcaaggatgatcaacttgttcaggttagaactgagttacatgataaagaacaatatatagaatcaatgcagagtgaattagagagcataagattcagatgctcgcaagctgaatctgtgttgcgaaggatggctgagctcactggcgatcttgctagttccgtgaaagctggagaaatggacatttatgcattactggatgatgaaatttcaagcaccagtacagtccttgagtccaatttgcacaagcataatcaactggaggctgacatagagatgttaagagaatccttacggcataaggacatggacttaagagctgctcatgaagcacttgatgccaaagatcaagaactgaaggcagtagttggaaagtgggatttcaaggagaaggaactggatgaggtggaagagttacagaaagatcccattgacatgaaggaactccctgttctttctaacgagacaacagggggcagcattacaggagagatggagctcaagaagcttcaaattgaagctgccgaggtggaggcacttgctgctactactgcactaaagaagcttgcggatatgagtaagaaatacttgagatgccgcaaagctgattctgggattggtttggttgcatcagaaagtgcaaacattggtaaagcgaattctaggatggaattaaacaacaagatggatgtgatttttgaagctaaacaagaaattgttagactattttcattgacaaaagagctcatcactgatgacgcaataaatgatgctgaggaacgataa

SEQ ID NO: 16 - DNA, 2316 bp, synthetic construct
  misc_feature 1..2316: note = Z.mays CDS (GRMZM2G104357_T01)
  source 1..2316: mol_type = other DNA
atgcccctctcctccaccacctcgccgtcggcgggggcggccgccgccgccgcagtgcgcaccgcttcgccgcctcgccgcatcgccacccacgttttgttcaggcagaagctaggcattccggcggggttccaggctcaacatgtgaaatgtttacctcatttgatcagatctattgtaagaggtgctagatcagatatcactgacggtgacaatggaacaactgagcccgcgagggaactattggagcgtctgtttgccaagacaaagagtctagatccaagcgcttctcagggtagggaactgagcatgagcattgaggtcctgaagactgagtttgaggctgccttatcaatcctaaggaagaaagagaaggatcttcgtgatgcggagaagaaagtctccgtggataggtcaaggttgaaccagacgaagcaggacctcgatcagagggaggaggacatcatcaaagcatactcgaggcaacatgaaatggagaaagcactgatgaaggcgagcagggatttgactctacaagtccgacagatcaataacctgaaggttatgatcgaggaacaagacaaaaaacttgttagttcacaagacgcactttctaagaaggttattgaagtggataagcttaaacaagagatgctgaagaagaatgatgaagtagctttgctgcattcagagatcgagtccaaggaacaagagcttcttgtagctaatcaggccattgcacgtcaagaagcaacaattagggagcttcgaagtgaaactaaaagaaaggaaactgaggttgagagattaaatgaattggcgaaagctaatgaagacaaactgaaatttgcagaacaggaacttgagaagcagaattcaggatggattgcagcacagcaagagttaaaggaattggcacaaatggcattcaaggataaagatgatatcaagaatacaatcaatgacttcaaacgggtgaggtatttgctggatgctgtgcgttctgaactaatagcttcaaaagaggctttaaccttctcacgcaagcaagtagaagatcaagcggcacagttgagtaaccaagtgcaggaactcacagaccaaaaggcactgattatttcttatacccggaatctggaagctgcccagctggagattcaaggaaagtcaaatgagctcagtactgtacaatctcgttgtagtgaacttgaatctcagttacttgaggaaacggagaaggttgagttcctagaggctatgttaaccaaagaaagggagatcttggaacagaaaactaaggaagtggcgttccttcaagaggaggtagttcagaaggagaaggattacttcaattcacaaaagcttgttgaaacaaaagagactgagctgttagaggcgaggcatgaagtcgaagatatgaaattgaaggtggattccatacaatttgctgttcgagagaaggatttggagcttctggaggcacaaagaaaacttgatgaagttaacagcgaagttgttgaacttcagcagctgataaatagcaaggaggatcaactggtccaagttagaactgaattacaggataaagagcaatgcatacaattgatgcaggatgaattggataagatgagattaggacgctcgcaagctgaatctgtggttcaaaagatagtcgagcttactagcaatctcataggttctgtcaaaggcgaagaattcaacatttataacttgctggatgatgaaattttaagcacgagcacagcccttgagtacagtttgcataagcataaccaactggaggctgacatagacatgttaaaagaatccctgcgacagaaggacatggatctgactgctgcttataaagcgcttgacgccaaagatcgagagttgaaggcagtagttggaaggttagatgttagggacaaggaactagacaagttggaagagctatccatagacccctatggcaccaggaaactgtctagagttgctgatgaggcaaccgaagacaacattgctggtgaagcggagctccaaaagcatgagatggaatctgtggagatggaggcactagctgctagcactatgttgaagaagcttgcggatgtgactaagaaattcttgagaagtggtagaactgattctggtaccaatttagattcaaacgttagtgaaggtgcttctgaattggaaccacaaaggaaacttaatgtgattctcgaggctaaaaaggagattgtcgggctattttctttgacagaagagctcgtcactggtgctcaaacgaaggacgatgatgaggaaccatag

SEQ ID NO: 17 - DNA, 3094 bp, synthetic construct
  misc_feature 1..3094: note = Z.mays (GRMZM2G104357)
  source 1..3094: mol_type = other DNA
gctgcactccatacgcggccgggagctcccccggttccaccacctcctcacctcatccgtctgaagccgccgcttttgcctcgacctcgcccgcagtccgcgatagcgcgggttgccacccgtccgatcgcgcccggacgaccgcgaccccctcccctcatgttccgcggccatgcccctctcctccaccacctcgccgtcggcgggggcggccgccgccgccgcagtgcgcaccgcttcgccgcctcgccgcatcgccacccacgtgagcattcttgtaccttcactgattcttataaattatcatgctgtgttgtcacggtatcgggttgctgagctcagtggcgattgtgttcttggatgccttccgaattgcggtctcgtgtagacgtccagtacctttttttagttgtgtttaactgaagataaaagatgaattttcggattcctaacccgtactcttttggtacctttctcttcgccggagtcgattttatggtggtaggttcgtgcgcagttcagattaatcaaatccattaccaaaattcggtcgagtgaagatagctgtgtatttttctttagcccgtctgaactcttctaattaaagcacaattcgtgtgcattatcctaatagcgcccccacgcgttttttttccttgtaaactcccaggttttgttcaggcagaagctaggcattccggcggggttccaggctcaacatgtgaaatgtttacctcatttgatcagatctattgtaagaggtgctagatcagatatcactgacggtgacaatggaacaactgagcccgcgagggaactattggagcgtctgtttgccaagacaaagagtctagatccaagcgcttctcagggtagggaactgagcatgagcattgaggtcctgaagactgagtttgaggctgccttatcaatcctaaggaagaaagagaaggatcttcgtgatgcggagaagaaagtctccgtggataggtcaaggttgaaccagacgaagcaggacctcgatcagagggaggaggacatcatcaaagcatactcgaggcaacatgaaatggagaaagcactgatgaaggcgagcagggatttgactctacaagtccgacagatcaataacctgaaggttatgatcgaggaacaagacaaaaaacttgttagttcacaagacgcactttctaagaaggttattgaagtggataagcttaaacaagagatgctgaagaagaatgatgaagtagctttgctgcattcagagatcgagtccaaggaacaagagcttcttgtagctaatcaggccattgcacgtcaagaagcaacaattagggagcttcgaagtgaaactaaaagaaaggaaactgaggttgagagattaaatgaattggcgaaagctaatgaagacaaactgaaatttgcagaacaggaacttgagaagcagaattcaggatggattgcagcacagcaagagttaaaggaattggcacaaatggcattcaaggataaagatgatatcaagaatacaatcaatgacttcaaacgggtgaggtatttgctggatgctgtgcgttctgaactaatagcttcaaaagaggctttaaccttctcacgcaagcaagtagaagatcaagcggcacagttgagtaaccaagtgcaggaactcacagaccaaaaggcactgattatttcttatacccggaatctggaagctgcccagctggagattcaaggaaagtcaaatgagctcagtactgtacaatctcgttgtagtgaacttgaatctcagttacttgaggaaacggagaaggttgagttcctagaggctatgttaaccaaagaaagggagatcttggaacagaaaactaaggaagtggcgttccttcaagaggaggtagttcagaaggagaaggattacttcaattcacaaaagcttgttgaaacaaaagagactgagctgttagaggcgaggcatgaagtcgaagatatgaaattgaaggtggattccatacaatttgctgttcgagagaaggatttggagcttctggaggcacaaagaaaacttgatgaagttaacagcgaagttgttgaacttcagcagctgataaatagcaaggaggatcaactggtccaagttagaactgaattacaggataaagagcaatgcatacaattgatgcaggatgaattggataagatgagattaggacgctcgcaagctgaatctgtggttcaaaagatagtcgagcttactagcaatctcataggttctgtcaaaggcgaagaattcaacatttataacttgctggatgatgaaattttaagcacgagcacagcccttgagtacagtttgcataagcataaccaactggaggctgacatagacatgttaaaagaatccctgcgacagaaggacatggatctgactgctgcttataaagcgcttgacgccaaagatcgagagttgaaggcagtagttggaaggttagatgttagggacaaggaactagacaagttggaagagctatccatagacccctatggcaccaggaaactgtctagagttgctgatgaggcaaccgaagacaacattgctggtgaagcggagctccaaaagcatgagatggaatctgtggagatggaggcactagctgctagcactatgttgaagaagcttgcggatgtgactaagaaattcttgagaagtggtagaactgattctggtaccaatttagattcaaacgttagtgaaggtgcttctgaattggaaccacaaaggaaacttaatgtgattctcgaggctaaaaaggagattgtcgggctattttctttgacagaagagctcgtcactggtgctcaaacgaaggacgatgatgaggaaccatagcatcataagttcataactgaacatataactgcatcatgaatgttatgcatgtcaactggaatgacgcagctgaaattgttaccatcgtttcctttccttttggggctcaggatttctttatgttcttttggttcatggatggcatcgcaaaatgaaaaaaaaagattgtttagattttggttaaaaggatgatgattgtgc

SEQ ID NO: 18 - protein, 771 aa, synthetic construct
  REGION 1..771: note = Z.mays (GRMZM2G104357_T01)
MPLSSTTSPSAGAAAAAAVRTASPPRRIATHVLFRQKLGIPAGFQAQHVKCLPHLIRSIVRGARSDITDGDNGTTEPARELLERLFAKTKSLDPSASQGRELSMSIEVLKTEFEAALSILRKKEKDLRDAEKKVSVDRSRLNQTKQDLDQREEDIIKAYSRQHEMEKALMKASRDLTLQVRQINNLKVMIEEQDKKLVSSQDALSKKVIEVDKLKQEMLKKNDEVALLHSEIESKEQELLVANQAIARQEATIRELRSETKRKETEVERLNELAKANEDKLKFAEQELEKQNSGWIAAQQELKELAQMAFKDKDDIKNTINDFKRVRYLLDAVRSELIASKEALTFSRKQVEDQAAQLSNQVQELTDQKALIISYTRNLEAAQLEIQGKSNELSTVQSRCSELESQLLEETEKVEFLEAMLTKEREILEQKTKEVAFLQEEVVQKEKDYFNSQKLVETKETELLEARHEVEDMKLKVDSIQFAVREKDLELLEAQRKLDEVNSEVVELQQLINSKEDQLVQVRTELQDKEQCIQLMQDELDKMRLGRSQAESVVQKIVELTSNLIGSVKGEEFNIYNLLDDEILSTSTALEYSLHKHNQLEADIDMLKESLRQKDMDLTAAYKALDAKDRELKAVVGRLDVRDKELDKLEELSIDPYGTRKLSRVADEATEDNIAGEAELQKHEMESVEMEALAASTMLKKLADVTKKFLRSGRTDSGTNLDSNVSEGASELEPQRKLNVILEAKKEIVGLFSLTEELVTGAQTKDDDEEP

SEQ ID NO: 19 - protein, 763 aa, synthetic construct
  REGION 1..763: note = O. sativa (LOC_Os02g09340.1)
MPPLSPSSSPPATAAAVLRCGSPSCRPVTHELFRQKLSFMVSFQAQHMRCAPHLIKSVVKGIRANITDGENGATEPARELLERLFAKTQRLDTSASQDSELSMSIDVLKSEFEAALSTLRKKERDLRDAENRVSVDQVRLNRAKKDLDQRERGINRAYARQQEMERSLGKASRDLVLQVRQIDNLKLLVDEQDKKIASSQDLLSQKVTEVEKLKQDMLKKNEEVTLMRSEIKSKEQLLLEANQAAEQQEATIKELRSEIKRKEIDFSRSNELRKANEQKLKIAEQELERQNMGWLAAQKELKEVAQLACKDMDGIKDTVSDFKRVRSLLDAVRSELIASKEAFSSSRKQIEDQAVQMQKQVQELSGQRLLLSSFNQNLEAARLEIQGKAKELNAAQSRCHELESLLLQEKEKVESLEAVLTKERESLEEKTKEVELLQKALVQKENEHSNSLKLVEIKESELLEARNEVQDMKSKVESIQIAVQEKDSELSETQRRLAEVNSEVVELKQLLDSKEDQLVQVRTELQDKEQHIQTLQNKLDSMKFSCSQAESVVQKIAELTGNLASSVEGEEMDIYALLDDEISSTGTALKSNLHKHNQLEADIEMLKESLHQKDMDLRAAHEALDAKDQELKAVMRRWDVKEEVDKLEGFLKDPSDIKRPSDFSVHMGLQNLQTEAAEVEALAATTTLKKLADMAKGFLRSGKTDSGINLVASPSVNSTRIVSKTKPNKEMDMILDAEKEIAGLFSLTEQLITEAGIDVAHQA

SEQ ID NO: 20 - DNA, 3580 bp, synthetic construct
  misc_feature 1..3580: note = O. sativa (LOC_Os02g09340)
  source 1..3580: mol_type = other DNA
gcagtccacacgcgtccggttcggcctccacatcatcatctcctctcctctctcgtctccgcgcgccgtgagccgtccgatctcgcgcgggcggccgtgatccgctccctcccccccacccacgtgctaggccgccatgccacccctctccccttcctcctcgccgccggcgacggcggcggcggttctccgctgcggctccccgtcgtgccgccccgtcacacatgtgagcatcccttcccatctcctcgtatctcctctctcgttgctcgttgttcgttgttggtggtgtgtgttctcgttcggtttccgattccgagctcgtctactagcggtttgaaggggacggaggatcggattttttttggttttctagttcaagttgtgctgcctggtagtggtagttttttaattaggagttttttggctgaaattggtttgacggtggtcaattccggtacatctttaaactaatcttgataacggagaacctaacgagaactattagcgattgtagtttgtgtgtggcgatagatttgggatcaaaattcatccgctgcgctatgtcgctttgctgaagattgaaactatgtgtgaataaatatagttctgaatttttagttcagagttaactttggcatactatatcatttcagtcctttggttcaccgggagaattttcatgtacaaataaaatccccatttttgcagataggttcactaggaggtgtcctgttgcaacttttttctagcagtctactgatgcacactgttttctgcaatcccgacaggagctgttcaggcagaagttgagtttcatggtgtcatttcaagctcaacatatgagatgtgctcctcatttgatcaaatcagttgttaaaggtattagagcaaatatcactgatggcgagaatggagcaactgaaccagctagggagctattggagcggctgtttgcgaagacgcaaaggttggataccagtgcttcccaggatagtgagctgagtatgagcattgatgtactgaagtctgaatttgaggccgccttgtctaccttgaggaagaaagagagggatctccgagatgcggagaatcgggtttcagttgatcaggtacgcctgaaccgggcgaagaaggatcttgatcagagagagcgtgggatcaatagagcatatgcaaggcaacaggaaatggagagatcactgggtaaggcaagtagagatctggttttacaagtgaggcagatcgataacctgaagcttcttgttgatgagcaagacaagaaaattgccagctcacaagatttgctttctcagaaggtaactgaagtggaaaagcttaagcaagatatgttgaagaagaatgaagaagtaaccttgatgcgttcagagatcaagtccaaggaacagctgcttcttgaagctaatcaggctgctgagcagcaagaagcaacaattaaggagctccggagtgaaattaaaagaaaagaaattgatttttccagatcgaatgaattgagaaaggccaatgaacagaaactaaaaatcgccgagcaagaacttgagaggcagaatatgggatggttagcagcacagaaagagttaaaggaagtggcgcaactagcatgcaaggatatggatggtatcaaggatacagtcagtgacttcaaacgtgtgaggtctctgctggatgctgtacggtctgaactaatcgcttcaaaagaggctttctcctcctctcgaaaacagatagaagatcaagcagtgcagatgcagaaacaagttcaagaactctctggtcaaaggctattgctttcatctttcaaccagaacttggaagctgctcggttggagattcaaggcaaggcaaaggagctcaatgctgcacagtctcgctgtcatgaacttgaatcactgttacttcaggaaaaggagaaggttgagtctctggaagcagtgttaacaaaagaaagagagagcttagaagagaaaaccaaagaagttgagttgcttcaaaaggcgctcgttcagaaggaaaatgagcacagcaattcattaaagcttgttgaaataaaagaatctgagctgttagaagcccgaaatgaagtccaagatatgaaatcaaaggtggaatctatccaaatagctgttcaggagaaggattcagagctttctgaaacacaacgcagacttgctgaagtgaacagtgaagttgttgaactaaagcagctgctagatagcaaggaagatcaacttgttcaggttagaaccgaattacaggataaagaacaacacatacagacactacagaataaattggatagcatgaaattcagttgctcacaagctgaatctgtggtgcaaaagatagctgaactcactggcaatcttgctagttcagtagaaggcgaagagatggacatttatgcattgctggatgatgagatttcgagcacaggtacagccctcaagtccaatttgcacaagcataatcaactggaggctgacatagagatgttaaaagaatccttgcatcagaaggacatggatttaagagctgcccatgaagcacttgacgcgaaagatcaagagctgaaggcggtaatgagaaggtgggatgtgaaggaggaggtagacaagttggaagggttcctgaaagatcctagtgacatcaagagaccttctgatttttccgttcatatggggctccaaaatcttcaaactgaagctgcggaggtggaggcacttgctgctactactacattgaagaaacttgcagatatggctaagggattcctgagaagtggcaaaactgattctggcatcaatctggttgcatcgccaagtgtaaacagtactagaattgtttccaagaccaaaccaaacaaggaaatggatatgattcttgatgctgaaaaggaaattgccgggctcttttcgttgacagaacagctcattaccgaggctggaatagatgttgctcaccaagcatagcttcagaacccagaaatgtatatcatattgcagttttgcaacatttagatagttgctgtgaagattcagctgaaattgttagcgttttcatttcttttgctttgggtctcatcgccctgtttgctgttggattgctctgctccaagtgcgaaagggagacatcgatgttgatatgcctcttactgtttacatgatatattgcatctgctgaaatcctagaaaaaaaatgataaatttgtgatccaaaaccccttctctagtcagccagtggcggatttgctgacaacctaggcagctgcctgtaattcatattctccaaaactccttcagaccggagttaatgaagatatccaaagcctgtaaaattgttgctgattttggagaggtgagaaatagtgaaggattttactttctggacggataccaggttgcccttgattttggtcagctttcgacatgtagatttgctgaattatatctgtttttctcttctctgtagtttcccatcggagagctgttgattctcatcatgttatccatatgttaacctggaaaaattgtacagaatttgctgctcagctttagtg

SEQ ID NO: 21 - DNA, 2292 bp, synthetic construct
  misc_feature 1..2292: note = O. sativa CDS (LOC_Os02g09340.1)
  source 1..2292: mol_type = other DNA
atgccacccctctccccttcctcctcgccgccggcgacggcggcggcggttctccgctgcggctccccgtcgtgccgccccgtcacacatgagctgttcaggcagaagttgagtttcatggtgtcatttcaagctcaacatatgagatgtgctcctcatttgatcaaatcagttgttaaaggtattagagcaaatatcactgatggcgagaatggagcaactgaaccagctagggagctattggagcggctgtttgcgaagacgcaaaggttggataccagtgcttcccaggatagtgagctgagtatgagcattgatgtactgaagtctgaatttgaggccgccttgtctaccttgaggaagaaagagagggatctccgagatgcggagaatcgggtttcagttgatcaggtacgcctgaaccgggcgaagaaggatcttgatcagagagagcgtgggatcaatagagcatatgcaaggcaacaggaaatggagagatcactgggtaaggcaagtagagatctggttttacaagtgaggcagatcgataacctgaagcttcttgttgatgagcaagacaagaaaattgccagctcacaagatttgctttctcagaaggtaactgaagtggaaaagcttaagcaagatatgttgaagaagaatgaagaagtaaccttgatgcgttcagagatcaagtccaaggaacagctgcttcttgaagctaatcaggctgctgagcagcaagaagcaacaattaaggagctccggagtgaaattaaaagaaaagaaattgatttttccagatcgaatgaattgagaaaggccaatgaacagaaactaaaaatcgccgagcaagaacttgagaggcagaatatgggatggttagcagcacagaaagagttaaaggaagtggcgcaactagcatgcaaggatatggatggtatcaaggatacagtcagtgacttcaaacgtgtgaggtctctgctggatgctgtacggtctgaactaatcgcttcaaaagaggctttctcctcctctcgaaaacagatagaagatcaagcagtgcagatgcagaaacaagttcaagaactctctggtcaaaggctattgctttcatctttcaaccagaacttggaagctgctcggttggagattcaaggcaaggcaaaggagctcaatgctgcacagtctcgctgtcatgaacttgaatcactgttacttcaggaaaaggagaaggttgagtctctggaagcagtgttaacaaaagaaagagagagcttagaagagaaaaccaaagaagttgagttgcttcaaaaggcgctcgttcagaaggaaaatgagcacagcaattcattaaagcttgttgaaataaaagaatctgagctgttagaagcccgaaatgaagtccaagatatgaaatcaaaggtggaatctatccaaatagctgttcaggagaaggattcagagctttctgaaacacaacgcagacttgctgaagtgaacagtgaagttgttgaactaaagcagctgctagatagcaaggaagatcaacttgttcaggttagaaccgaattacaggataaagaacaacacatacagacactacagaataaattggatagcatgaaattcagttgctcacaagctgaatctgtggtgcaaaagatagctgaactcactggcaatcttgctagttcagtagaaggcgaagagatggacatttatgcattgctggatgatgagatttcgagcacaggtacagccctcaagtccaatttgcacaagcataatcaactggaggctgacatagagatgttaaaagaatccttgcatcagaaggacatggatttaagagctgcccatgaagcacttgacgcgaaagatcaagagctgaaggcggtaatgagaaggtgggatgtgaaggaggaggtagacaagttggaagggttcctgaaagatcctagtgacatcaagagaccttctgatttttccgttcatatggggctccaaaatcttcaaactgaagctgcggaggtggaggcacttgctgctactactacattgaagaaacttgcagatatggctaagggattcctgagaagtggcaaaactgattctggcatcaatctggttgcatcgccaagtgtaaacagtactagaattgtttccaagaccaaaccaaacaaggaaatggatatgattcttgatgctgaaaaggaaattgccgggctcttttcgttgacagaacagctcattaccgaggctggaatagatgttgctcaccaagcatag

SEQ ID NO: 22 - DNA, 1500 bp, synthetic construct
  misc_feature 1..1500: note = TaMRC 6A (cv. Kronos (4n)) promoter sequence
  source 1..1500: mol_type = other DNA
ttaacctgaaaatctaaaaagtggccgcgcactttttagtcgaaccgagcggccggctctcacagcgtatgcatgtggtaatttatttgtctgctagtgcatgtaggcgtgacattaaatacgttcacactgctcttttagtttaagaaagacagatccatctgcatttattttgggtttttaaaaattcaaaaagctatatctttcaaaccgcgcgtcggaattcaaatccgttttcaccattgaaatcctcgcgacgagatctttgaaactagatcccgcatgggtatattttgacgaatttttttcgatgccaactttggagctatatagtgcaactctattactgcaatgtgcaacttttattactacatcgtgcaacttttttccaaaactaatgtttggagctgcaccttcgtatgaggttacaacctagcaaccacgacaactttgatgtgcgactagtctattgcctcgacgaaagtcgatgtgcaacctcctcttgtaatgtagtctagtcgcatacacattgatgtttagttggcttgtaatgtagtctagttgcacatacattgatatttagttggcttgtaatgtagtctagttgcacacacattaatgtttagttggcaggaagactaattgcacacgcatatgattagttggcttgtaatttagtctagttgcacacacactgatgtttagttggcaggacactagttgcacacacatatgctcagttggcgtggcaatctagtctagttgcacacacattgatgtttagttggcaggacttttttggcacacacacatatgctcagttggcgcgcaatctagtctagttgcacacacattgatatttagttggcagaaagactagttcgtcgaaacatccccatgcgggatatagttttgaagagcacgtcgcgaggattccagcggtgaaaacggatcttaattccgacgcgcggtttggaagatatagctttttgaaaatttaaaaaccgaaacaaatgcatatgtgatctgttttttccaactgattgtgaccggtgtgaatgtattaaatgctaaaagatacatgcgctagcggacaaaaattacacacatgcatgtctatacagagcagacgctagcgaataaaaaatttctattttaggccggccgctcgcgcatggcagcgagcagccggccgctacgtagactcgtctttttttaaggcaaccaaagtgtaccttaatttttcatgtgttataaactcatacatttggaacagaaagaaaaaaggtagtaagacgagtgaacggagaagaaaagctgtagaacagtagaaggcaaacgagtaaacgacacagctctctctcacgcttctcgcgtggtcgacgttgcagtccacacgcggctgggcgcgccggttcaaccacacctcatctcccgcactccctctgcctcgtatctcctcgccttcctccgcaccccgcaggcgcattgccagccgtc

SEQ ID NO: 23 - DNA, 1500 bp, synthetic construct
  misc_feature 1..1500: note = TaMRC 6A (cv. Cadenza (6n)) promoter sequence
  source 1..1500: mol_type = other DNA
ttaacctgaaaatctaaaaagtggccgcgcactttttagtcgaaccgagcggccggctctcacagcgtatgcatgtggtaatttatttgtctgctagtgcatgtaggcgtgacattaaatacgttcacactgctcttttagtttaagaaagacagatccatctgcatttattttgggtttttaaaaattcaaaaagctatatctttcaaaccgcgcgtcggaattcaaatccgttttcaccattgaaatcctcgcgacgagatctttgaaactagatcccgcatgggtatattttgacgaatttttttcgatgccaactttggagctatatagtgcaactctattactgcaatgtgcaacttttattactacatcgtgcaacttttttccaaaactaatgtttggagctgcaccttcgtatgaggttacaacctagcaaccacgacaactttgatgtgcgactagtctattgcctcgacgaaagtcgatgtgcaacctcctcttgtaatgtagtctagtcgcatacacattgatgtttagttggcttgtaatgtagtctagttgcacatacattgatatttagttggcttgtaatgtagtctagttgcacacacattaatgtttagttggcaggaagactaattgcacacgcatatgattagttggcttgtaatttagtctagttgcacacacactgatgtttagttggcaggacactagttgcacacacatatgctcagttggcgtggcaatctagtctagttgcacacacattgatgtttagttggcaggacttttttggcacacacacatatgctcagttggcgcgcaatctagtctagttgcacacacattgatgtttagttggcagaaagactagttcgtcgaaacatccccatgcgggatatagttttgaagagcacgtcgcgaggattccagcggtgaaaacggatcttaattccgacgcgcggtttggaagatatagctttttgaaaatttaaaaaccgaaacaaatgcatatgtgatctgttttttccaactgattgtgaccggtgtgaatgtattaaatgctaaaagatacatgcgctagcggacaaaaattacacacatgcatgtctatacagagcagacgctagcgaataaaaaatttctattttaggccggccgctcgcgcatggcagcgagcagccggccgctacgtagactcgtctttttttaaggcaaccaaagtgtaccttaatttttcatgtgttataaactcatacatttggaacagaaagaaaaaaggtagtaagacgagtgaacggagaagaaaagctgtagaacagtagaaggcaaacgagtaaacgacacagctctctctcacgcttctcgcgtggtcgacgttgcagtccacacgcggctgggcgcgccggttcaaccacacctcatctcccgcactccctctgcctcgtatctcctcgccttcctccgcaccccgcaggcgcattgccagccgtc

SEQ ID NO: 24 - DNA, 1500 bp, synthetic construct
  misc_feature 1..1500: note = TaMRC 6D (cv. Cadenza (6n)) promoter sequence
  source 1..1500: mol_type = other DNA
cgctggtaatcaccagcgcagctaaccaccccatgagcttctagttactggttagaaaagggatcgcgaccaactagaggtgactgagccggtcttctccattatttctttctttttttcttttctttctatattttttttcaaatacttgttcaaattttttcaaatacttgttcaaattttttcaaatacttgttcaatatttttatatacatgatcaacatttttacaaatacttgttcaatatttttatatacatgatcaatatttttttaaatacttattcaacatttttttcaaatagttgttcaattttttttgcaaatgcttgattatcatttttatatacatgatcaacattttttaaaatacttcttcaacagttttcaaatacctattcaacagttttcaaatatttgttcaacatctttcaagtacttgttcaacattttttcaaatgcttgattttatatgcatgatcaacattttttcaaatacttcttcaacatttttaaatacctattcaacagataaatgggtgcatgcacctgtcggcctagaggaagcagactgggtgccaatcgaccagcagtgggccgactggggccaatcggccagcagtgggccgactggatccaatcggccggcagtgggccgactgaggccaatcggccagcagtaggccgattggatccaatcggccagcagtaggccgactggatccaattggtcagcagtaggccgactgggtaatattttaaaaaaatataattatggcgtaatatttctgaaatttaatataaaacatgtattatttaaaaaattagtccgactatgagtagctctgctagtttaaatttatgtcacctatgtttgaactatgtttgaatttgatgtttgagctatgttgagatcaaatatagttggtcgtttcagaattttacatcttcgttttggaccatctgttggagttgctcttttacatcaccattttggactatctggtggagttgagccgttttcaaagatgtaaaaagcaatttttgatgatgtaaatttttacatcaccggtttggagcaccaaaatatacatcatctattggagatgctcttaccctagtattccgttccgagtcaaaatagtccctgataattttcatgtgttataaactcattctttttaaagtcaagactgtccctaacaattttcatgtgttatataaactcattttttaaggcagccaaggtgtaccttaaattccatgtgttataaactcataaatttggaatagaaagaaaaaaagggaagaccagtgaacggaggagaaaagcagtagaaggcaaacgacacagctctctctcacgcttctcccgtggtcgacgttgcagtccacacgcggctgggcgcggcggttccaccacctccctcatctcccgtactccctctgcctcgtatctcgtcgccttcctctgcaccccgcaggcgcattgccagccgtc

SEQ ID NO: 25 - DNA, 1500 bp, synthetic construct
  misc_feature 1..1500: note = HvMRC promoter sequence
  source 1..1500: mol_type = other DNA
cacctcatcccattccccagcatggaccttctcctcaaagtacttggtgttgaagtagaatcctgactcttgttcaagcctgcagcatcatgcattgtcaaaatcacataaaaatattgcaatactccattgcactaaacataatagatggaccagagctcgaaggggaaaaagaaaaatggtatggcgctcaggaagacctcggcaattcaattcttgagctcacaacattggcccagtactgccaacccgagttgtctacctgaggcgtcacgcatgactgtactgacgacaatacagaagacaagtgaaaacagagtttcagactaatccatttgtttcgatggatggttggatacccagttgtgcgctcaatgtccaaatttgacagtggcttcgaacaatactgcaatccaggtaaatgccatctacacttttccatttactgatgttttactcggaaaaggcaccgcacaggagacaacaatgccaccaatcactacaatctaggtaaattcagtgtggtaaactttaataagtattttaagcttcttgtaacatcatatttcttccattcgaaagtaagttcatgaatccttgtgatgattttgtgggtgttctctgccacactgaatagaaaatctgcaaaaatagaaaagaacaaattctaaggtgtgtgtgtgtaactgaagaacctcgtcgggatcacttgtccggcgttgcagctggagccgcctcctcggcacgaggcttctctgcgtccgccatggcttcctgccacaagtggtgaaacacggtgacgacgataggaagggaatcaacatcgccatagcctcgagccctcgacacactcccttggcttgtgattcccatcgtggtggagatcttgaatccggcgacgtcgcaggagaagagagacgcgacgagcggggggaggggtcgcggcgagcggggggaggggtcgcggcgagcggcgggaggaggcgcggcgagcgggggaggggtcgaggcgcggtggcgggaggagttgcggcgagcggggggaggaggcgcggcgagcgagggaggagtcgcgacgagcggcggggaaggggtcgaggcgaacggggggaggggtcgcggcaagcatgccgaccaggaggagggggtctattcgtgagacgtggagcgtgagtattgattagcgctaacgaaaaccgttttcgttaacggagtatttagacccttgattaagcgattagacggttaaaattatgatttggatctgtcctttcgtgtttttattatttgaaatataaaggaaaaaagcaagactaatgaacggagaaggaaagcagtagaaggcaaacggcacagcgctctctcacgcttttcccgtggtcgacgttgcagtccacacgcggctggggtcggctggttccaccacctcatctccctgactccctctgcctcgtatctcgtcgccttgctccacaccccacaggcgcattgtcagccgtc

SEQ ID NO: 26 - DNA, 1500 bp, synthetic construct
  misc_feature 1..1500: note = Brachypodium MRC promoter sequence
  source 1..1500: mol_type = other DNA
tcccataatttttgtcgtgattttagttcaaatttgaattaaaaccagcacaataattatgggactgaaggagtactacgaagtagtaaaaaatacttcctccgatctatattacttgtcgctgatttagtacaagggagtagtattttttattagagtcatcatgatagtatatatgtttaccgtatgtgtaaatattaatacaatttaacataaatttagtcaaaatttaataagctcgtttatgacaaaactaaaacgtttgtcctcgtctgtgcgcacgtgcgatatcggatctactgtaagtcatggaataaccccccgccaccttctcatgtgaaacgctatgttcccccttcccagtagcccgtttccccaatttccatcacaattgtcgttcccagctctggcgctgccccaggctatggcgcccgaccctcctgctcaggtgttagtagccatggatttctgttgttcctgccaggggaagtggagcgcatccgcgaaggctcccctccacaccccctcgaagcaccgccatggtggcacctcgaggcgatgcgcgccacgacgcgcgggttccgctctaccttcactagctactcgatcatgcgactgctattcgaatgattcatgcgctcactttaaaccctccaatcttgacattaggggcgggcattcggtcatgactgaaagttcggtcttctaatttaagtcttttttcgattcggtccttaaaatataatacccgaactttactgagaaacgtcgatgaccgaactttagaaagatgggtgataattccaaaacaaaacgtgatttggtaccaattttagcaacatttcacttatacttctcacaaattttcttattaacatgggcattggagattaggaaagtgaaacagtcgattgcctctcttgtgccatgatttttgcatgcggaatagttgatgcctagattgtatcgtgaaaatgagttggtttttacaaagcggaaaagtttggtcctatttggtctttcagtcttttgtggattgatacccgaattaatagaataaatttcggtctataaatttttctatccaaatatctaatcggtcttttcgatctcggtctattcggtttcggtcctcggtttttatgcccgcccctacttgacatatactttagaatactttatgtataaacagttttgctcgatgctaagtggcagtgttccgtgctttgtcgaccccacccaagatcctagcttgtgtctaaacatttgagagagagagcaagagatttccctcgttttctttaaaaataaaaggaaaatagataattaattaattatttttcttaaggaaaataactagtacacgattatattgttttttaggaaaacgacataattataggagagaaagcaaacaacacgggtctcacgcttctctacagtcgactaggctcggagtccacacgcggctgctcggcccgcttccacccctacatcccc

SEQ ID NO: 27 - DNA, 1500 bp, synthetic construct
  misc_feature 1..1500: note = Z.mays MRC promoter sequence
  source 1..1500: mol_type = other DNA
tatctatggctcgtacttttctataggaaagggtcagcaaggacattatgcacatattaaggcattgtttaggacaactctaactacatgacattttgtagagttggagaagtaggtcattggatgctttagaaaatcgtggagctctgtaaacatatacaagacatttagataagtcattttgtttattatttagattaaaaatatttttaaaactatttaaattgatattataaactatagctctacactggaggtttaacctggagccatctcaaacctgccctaaattatgtacatagttcttttacatgcaatgcacttattgaatcacgataatctattttagacctaacattctcttacatgtcccttcgtgcacagaatatggtgaatgtggtgaagagtttttttttttgcaaaaaatctgtccacatgatttagtcgctttagatttgttctaagtgaaactatttaaattttgaccaacaatatatatagttaaattatgttatactaaaataattatatattatgatagttcaagtcatgatggatctggcgactttacttttatattgtaaaattttataaaacattcggtataaccgatcaaaagttaatagtgattgacttaggacaaatctaaaacagctaattcatatggacagtgtgagtacaaagcaataagggaactagggaaggtgtgtgaggatgttggatcttagatgagtaactctgtgcaccaaagatctatatgcacttagggggtgtttggtttgtagtgtctaatttttagttccaccattttgttttatttgtccctaaattatcaaatatgaaaactaaaatagagttttattttcagtatttgataatttatggactaaaatgaaataaaatgaattgactaaaaattagtccctagaaaccaaacatctcctttggtttctagggactaatttttagtcactatatattttttattttagtttctaaattgtactttccatatttgacaatttaaagattaaaataaaaagactaaaaattagtctttacaaaattaaacatctctttaaggctttatctgaataccctcgtattcaccctaatccacgtgtattgaggtggattgaaatgtaaattagtttaatttacgcttcaatttatcttaataaatgtgattgagataaatacgagagtagtaagccataatatgttttcattagatgtaggattgttttttcgcgtggacgctgcatgatgttcgtctgacttggtaagccaggtatggacggaccacggatttagcaaatttacaagaaaattccctctcctaacaaatacgtaggaaatctcggaaatacgcagaaaagaggacgtcaaaaggcaattttcttttctaagatagggaaaaagtgtgaaaaagacaatttctcatcagaaaatggaaaacacaagtctccagtgctcctcgcgtgatcgcatccacgcttc

SEQ ID NO: 28 - DNA, 1500 bp, synthetic construct
  misc_feature 1..1500: note = O.sativa MRC promoter sequence
  source 1..1500: mol_type = other DNA
cagccgccggcctccgctcgccgcgactgccgtcctccgcttgccgcggccgctcgtcgcggccgccggcccccgcgcggcgcaacccgccgcccccgctcgccgtggctgctggcctccgcgcggcacaagctcgccgcgcccgccgtcctccgcgcagcgccgcccgctgcctccgcacggcgcccgcctcccgccgcgcagcccgccggtccccacgcagcgccgcctcccgccgacgctgccttcaccgccgtcccctccccgcttgctgccgccgcctgcttcttgtcaacgctagaaaagaaaagaggaagaggggaaggaaggggagaaagaataagggagtggctcacatatgggtcccggtgtcatagtcaaaatagagagggtagaatagagaggctgttggagtatacagttaatttgactagctaaatcagatggagagttggctatatgggtgttttaggagttcgatttggagaggctgttagagatactcttatgtgcaaaatatgtgcagttaccatgcatgttggtactgaagagccagagccattccaagaaaggaaggatcttttcaccacgtagaactccatttttctgacattttttaaaagaaagaatgaaggtgactaagggacaatattttaatatagcaggatgatatagagattctcgagggacaataaggttttacacttttacaaaggattctcaagatatacatatttcattcttgctagggttagtgtaggatggtgcagtgcacaccatgagaatcggagaatctgtatgtggtgcgaagaaaaactaagaacaccagaaacagaaaaaaaatgaagaacagaaacaaatccatgcaaaacttgtcattcagagagcaaattatgtctcagatatctctatttacctgctactcctgaagagactggacttgttacttcctttgacagaaatagcagaacaaaaatatgtcacctttttgcaactttcataagattttcttttctttttgcaaagaatttatgcatgctaaaaacaagggggtgagttaggaactcactgaaaagagcacagcctagatgagtaggtgcgctcccgtttttgggcccagatgaaggacaaggcccacctagaactcctacggtggaccgcgggcggttttgatccaacggccagggttcttcatcacccatccaacggtgacaatcccactaaaatccttccaaattttcggtttctttaaacatcttcgaattcaaatttctctcatcacgtagtacagcccagcatttcctgtcctcacgtacccccgaataaaaacgaaacggcaccagaacccagaacagcaagcaacacaaacccatcaaaacaaacaaaacaaaaagaaaagaaaaagaaaaaaaaagagcaagcgacacgagcgtcacgtgtcacgcttactcgagtcatcaatcatctactacaccccaccccactccactgcactgcact

SEQ ID NO: 29 - protein, 505 aa, synthetic construct
  REGION 1..505: note = TaMRC 6B partial (cv. Kronos (4n))
PVSNESRKTNEEKLKVAEQELEKQSLGWLAAQQELKELAQLAFKDTDDINGIITDFKRVRSLLDAVRSELISSKDAFASSRRQIEDQAVQLQEQVQELEDQRVLLMSYTHDLEAAKLEIQGKTQELSYAQSRCHELESQLLQEREKVESLEAELAKEKQSLEHRTEEVGFLQKELVQKENECTKSQELVKVKEFELLEARQEVQDMKLKVESIQLAVQEKDSELSDTQSRLTEVSSEIAELQQLLNSKKDQLLQARTELHDKEQHIETLESELDSIRLRCSQAESMVQRMADLTGDLASSVKAGEMDIYALLDDEISSTGTALESNLHKHNQLEADIEMLRECLRHKDMELRAAHEALDAKDQELKAVLRKWDVKEREVRELEELPDPSATNELAGFSSETTEDGIVGEMELPELQIEAVEVEALAATTALRKLADMTKDFFKHGKADSGIDLVASESQKISKCDPKMEVHKKTDVILEAEKEIVRLFSLTKQIVTDDIINDVEE

SEQ ID NO: 30 - protein, 505 aa, synthetic construct
  REGION 1..505: note = TaMRC 6B partial (cv. Cadenza (6n)
PVSNESRKTNEEKLKVAEQELEKQSLGWLAAQQELKELAQLAFKDTDDINGIITDFKRVRSLLDAVRSELISSKDAFASSRRQIEDQAVQLQEQVQELEDQRVLLMSYTHDLEAAKLEIQGKTQELSYAQSRCHELESQLLQEREKVESLEAELAKEKQSLEHRTEEVGFLQKELVQKENECTKSQELVKVKEFELLEARQEVQDMKLKVESIQLAVQEKDSELSDTQSRLTEVSSEIAELQQLLNSKKDQLLQARTELHDKEQHIETLESELDSIRLRCSQAESMVQRMADLTGDLASSVKAGEMDIYALLDDEISSTGTALESNLHKHNQLEADIEMLRECLRHKDMELRAAHEALDAKDQELKAVLRKWDVKEREVRELEELPDPSATNELAGFSSETTEDGIVGEMELPELQIEAVEVEALAATTALRKLADMTKDFFKHGKADSGIDLVASESQKISKCDPKMEVHKKTNVILEAEKEIVRLFSLTKQIVTDDIINDVEE

SEQ ID NO: 31 - DNA, 1546 bp, synthetic construct
  misc_feature 1..1546: note = TaMRC 6B (cv. Kronos (4n))
  source 1..1546: mol_type = other DNA
ccagtatcaaatgaatcgaggaaaactaatgaagagaaactgaaagttgctgaacaggaacttgagaagcagagtttaggatggttagcagcacaacaagagttaaaggaacttgcacaactggcattcaaagatacagatgatatcaatggtattatcactgacttcaaacgtgtgaggtctctgctagatgctgtacgctctgaattaatctcttcaaaagatgctttcgcttcctctcgcagacaaatagaagatcaagcggttcagttgcaggaacaagtacaggaactcgaggaccaaagggtattactgatgtcttacacccatgatttggaggctgctaaactggagattcaagggaagacacaggagctcagttacgcacagtctcgttgccatgaacttgaatcacagttacttcaggaaagggagaaggtcgagtctctagaagccgaattagccaaagaaaaacagagcttagaacatagaactgaagaagtaggctttcttcagaaggagcttgttcagaaagaaaatgagtgcaccaaatcacaagaacttgttaaagtaaaagagtttgagctgttagaagccagacaggaagtccaagacatgaagttaaaggtagagtctattcaattggctgttcaagaaaaggattcagagctttctgatacacagagcagactaactgaagtcagcagtgaaattgctgagcttcagcagttgctaaatagcaagaaggatcaactgcttcaggctagaactgaattacatgataaagagcaacatatagaaacactggagagtgagttggatagcatacggctcagatgctcgcaagctgaatccatggttcaaaggatggctgatctcactggcgatcttgctagttccgtaaaagccggagaaatggacatctatgcattactggatgatgaaatttcaagcacaggtacagccctcgagtccaatttgcataagcataatcaactggaggctgacatagagatgttaagagaatgcttgcggcataaggacatggagttgagagctgctcatgaagcacttgatgccaaagatcaagagctgaaggcagtacttagaaagtgggatgtgaaggagcgggaagtacgtgagttagaagagttaccggatcccagtgccacaaatgaacttgctggtttttccagtgagacaacagaggacggcattgtaggagagatggagctcccagagcttcaaattgaagctgtggaggtcgaagcacttgctgctacgactgcattgaggaagcttgcggatatgactaaggatttcttcaaacacggcaaagctgattctggtattgacttggttgcatcagagagtcagaaaatcagtaaatgtgatcctaaaatggaagtacacaagaagacggatgtgattcttgaagctgaaaaagaaatagttaggctcttctcattgacaaaacagattgtcactgatgacataataaacgatgttgaggaatgatagcttcaaactgaagcatgtagtcttc

SEQ ID NO: 32 - DNA, 1546 bp, synthetic construct
  misc_feature 1..1546: note = TaMRC 6B (cv. Cadenza (6n)
  source 1..1546: mol_type = other DNA
ccagtatcaaatgaatcgaggaaaactaatgaagagaaactgaaagttgctgaacaggaacttgagaagcagagtttaggatggttagcagcacaacaagagttaaaggaacttgcacaactggcattcaaagatacagatgatatcaatggtattatcactgacttcaaacgtgtgaggtctctgctagatgctgtacgctctgaattaatctcttcaaaagatgctttcgcttcctctcgcagacaaatagaagatcaagcggttcagttgcaggaacaagtacaggaactcgaggaccaaagggtattactgatgtcttacacccatgatttggaggctgctaaactggagattcaagggaagacacaggagctcagttacgcacagtctcgttgtcatgaacttgaatcacagttacttcaggaaagggagaaggtcgagtctctagaagccgaattagccaaagaaaaacagagcttagaacatagaactgaagaagtaggctttcttcagaaggagcttgttcagaaagaaaatgagtgcaccaaatcacaagaacttgttaaagtaaaagagtttgagctgttagaagccagacaggaagtccaagacatgaagttaaaggtagagtctattcaattggctgttcaagaaaaggattcagagctttctgatacacagagcagactaactgaagtcagcagtgaaattgctgagcttcagcagttgctaaatagcaagaaggatcaactgcttcaggctagaactgaattacatgataaagagcaacatatagaaacactggagagtgagttggatagcatacggctcagatgctcgcaagctgaatccatggttcaaaggatggctgatctcactggcgatcttgctagttccgtaaaagccggagaaatggacatctatgcattactggatgatgaaatttcaagcacaggtacagccctcgagtccaatttgcataagcataatcaactggaggctgacatagagatgttaagagaatgcttgcggcataaggacatggagttgagagctgctcatgaagcacttgatgccaaagatcaagagctgaaggcagtacttagaaagtgggatgtgaaggagcgggaagtacgtgagttagaagagttaccggatcccagtgccacaaatgaacttgctggtttttccagtgagacaacagaggacggcattgtaggagagatggagctcccagagcttcaaattgaagctgtggaggtcgaagcacttgctgctacgactgcattgaggaagcttgcggatatgactaaggatttcttcaaacacggcaaagctgattctggtattgacttggttgcatcagagagtcagaaaatcagtaaatgtgatcctaaaatggaagtacacaagaagacaaatgtgattcttgaagctgaaaaagaaatagttaggctcttctcattgacaaaacagattgtcactgatgacataataaacgatgttgaggaatgatagcttcaaactgaagcatgtagtcttc

SEQ ID NO: 33 - DNA, 1518 bp, synthetic construct
  misc_feature 1..1518: note = TaMRC 6B CDS (cv. Kronos (4n))
  source 1..1518: mol_type = other DNA
ccagtatcaaatgaatcgaggaaaactaatgaagagaaactgaaagttgctgaacaggaacttgagaagcagagtttaggatggttagcagcacaacaagagttaaaggaacttgcacaactggcattcaaagatacagatgatatcaatggtattatcactgacttcaaacgtgtgaggtctctgctagatgctgtacgctctgaattaatctcttcaaaagatgctttcgcttcctctcgcagacaaatagaagatcaagcggttcagttgcaggaacaagtacaggaactcgaggaccaaagggtattactgatgtcttacacccatgatttggaggctgctaaactggagattcaagggaagacacaggagctcagttacgcacagtctcgttgccatgaacttgaatcacagttacttcaggaaagggagaaggtcgagtctctagaagccgaattagccaaagaaaaacagagcttagaacatagaactgaagaagtaggctttcttcagaaggagcttgttcagaaagaaaatgagtgcaccaaatcacaagaacttgttaaagtaaaagagtttgagctgttagaagccagacaggaagtccaagacatgaagttaaaggtagagtctattcaattggctgttcaagaaaaggattcagagctttctgatacacagagcagactaactgaagtcagcagtgaaattgctgagcttcagcagttgctaaatagcaagaaggatcaactgcttcaggctagaactgaattacatgataaagagcaacatatagaaacactggagagtgagttggatagcatacggctcagatgctcgcaagctgaatccatggttcaaaggatggctgatctcactggcgatcttgctagttccgtaaaagccggagaaatggacatctatgcattactggatgatgaaatttcaagcacaggtacagccctcgagtccaatttgcataagcataatcaactggaggctgacatagagatgttaagagaatgcttgcggcataaggacatggagttgagagctgctcatgaagcacttgatgccaaagatcaagagctgaaggcagtacttagaaagtgggatgtgaaggagcgggaagtacgtgagttagaagagttaccggatcccagtgccacaaatgaacttgctggtttttccagtgagacaacagaggacggcattgtaggagagatggagctcccagagcttcaaattgaagctgtggaggtcgaagcacttgctgctacgactgcattgaggaagcttgcggatatgactaaggatttcttcaaacacggcaaagctgattctggtattgacttggttgcatcagagagtcagaaaatcagtaaatgtgatcctaaaatggaagtacacaagaagacggatgtgattcttgaagctgaaaaagaaatagttaggctcttctcattgacaaaacagattgtcactgatgacataataaacgatgttgaggaatga

SEQ ID NO: 34 - DNA, 1518 bp, synthetic construct
  misc_feature 1..1518: note = TaMRC 6B CDS (cv. Cadenza (6n))
  source 1..1518: mol_type = other DNA
ccagtatcaaatgaatcgaggaaaactaatgaagagaaactgaaagttgctgaacaggaacttgagaagcagagtttaggatggttagcagcacaacaagagttaaaggaacttgcacaactggcattcaaagatacagatgatatcaatggtattatcactgacttcaaacgtgtgaggtctctgctagatgctgtacgctctgaattaatctcttcaaaagatgctttcgcttcctctcgcagacaaatagaagatcaagcggttcagttgcaggaacaagtacaggaactcgaggaccaaagggtattactgatgtcttacacccatgatttggaggctgctaaactggagattcaagggaagacacaggagctcagttacgcacagtctcgttgtcatgaacttgaatcacagttacttcaggaaagggagaaggtcgagtctctagaagccgaattagccaaagaaaaacagagcttagaacatagaactgaagaagtaggctttcttcagaaggagcttgttcagaaagaaaatgagtgcaccaaatcacaagaacttgttaaagtaaaagagtttgagctgttagaagccagacaggaagtccaagacatgaagttaaaggtagagtctattcaattggctgttcaagaaaaggattcagagctttctgatacacagagcagactaactgaagtcagcagtgaaattgctgagcttcagcagttgctaaatagcaagaaggatcaactgcttcaggctagaactgaattacatgataaagagcaacatatagaaacactggagagtgagttggatagcatacggctcagatgctcgcaagctgaatccatggttcaaaggatggctgatctcactggcgatcttgctagttccgtaaaagccggagaaatggacatctatgcattactggatgatgaaatttcaagcacaggtacagccctcgagtccaatttgcataagcataatcaactggaggctgacatagagatgttaagagaatgcttgcggcataaggacatggagttgagagctgctcatgaagcacttgatgccaaagatcaagagctgaaggcagtacttagaaagtgggatgtgaaggagcgggaagtacgtgagttagaagagttaccggatcccagtgccacaaatgaacttgctggtttttccagtgagacaacagaggacggcattgtaggagagatggagctcccagagcttcaaattgaagctgtggaggtcgaagcacttgctgctacgactgcattgaggaagcttgcggatatgactaaggatttcttcaaacacggcaaagctgattctggtattgacttggttgcatcagagagtcagaaaatcagtaaatgtgatcctaaaatggaagtacacaagaagacaaatgtgattcttgaagctgaaaaagaaatagttaggctcttctcattgacaaaacagattgtcactgatgacataataaacgatgttgaggaatga

SEQ ID NO: 35 - DNA, 1328 bp, synthetic construct
  misc_feature 1..1328: note = TaMRC 6B upstream sequence (cv. Kronos (4n))
  source 1..1328: mol_type = other DNA
ctgaggcggccgtttcaggggcgccgatttctgtttggcaacgatgatggcgtcaagaggagcttgggtcgcggctcgggctttggtagtcggatcttcccggcgtgcccgaggtgctctttagggcacggtcggcgcaagtcctgcatatttcccttgtgaggctcgtcgtcaaagtcgaagttgtcggttgttggcgcgtgtggccatctgttggcatgtgccgtctttgctctgtgtagctgtttgcgagggcgacttgttggtggagctctatggtgaagtcggagtcgcccgttcggagataaccggtgatgacgatgacgcttgcgactccccgacgttcttctttgcagctcatgtttcatgtcggtggacaggttggccggtggtgcccatgttatatgggttgggttgtattggttttagcccggttttccgtcaattaaccgggcaattcttatttcttcttaatcaatgaaaatggcaagtcttttgcctcgtttcaaaaaaaataaaaataggagtaactgagctgcagttatcgtcgcccgcgagtggtacacatcaattgacataaagagctacgtcaacggagaattactccaaaatctccagcccaatcgacagagacgattcgttaccttgtcaccgccccttggcgttgcccgacgagtcctctcctgcccaccctgtctcttggttcgcaggaacgattcatcctgaccgcaaacgtccgtccgccggcaaggtcaacaacatcaagcacctcgcacagatcgcgtcgaccaacccgcgcctccttcgttcccatgtgtgcggtccatgatttttttttctcgtttgatgatggacacgaaagcgattactacagaccgaaggcccaataggcccaattaacagcagatggtggattttgttacgggagcagcggccaaatccatgtgcgcgacctgcatagcgaaggaagcccaggcaggattagaggaggatccaatggccagaaacacccactacttgagatccgacggctaaaaacggtaatggcctgagagagctagaagagtgcacggttatataatgtatttaaatttggaacagaaagaaaaaaggtagtaagacgagtgaacggggaagaaaagcagtagaaggcaaacgacgcagctctctctcacgcttctcccgtggtcgacgttgcagtccacacgcgggcgggcacgcggctgggcgcgccggttccaccacctcatctcccgcactccctctgcctcgtatctcgtcgccttcctccacaccccgcaggagcattgccagccgtccgatcgcgc

SEQ ID NO: 36 - DNA, 2000 bp, synthetic construct
  misc_feature 1..2000: note = TaMRC 6B promoter sequence (cv. Cadenza (6n))
  source 1..2000: mol_type = other DNA
ctggatctggtcgggcgtggctggatccgggcgttcttcgggggttttctgctgcagcctgttcgttgtctccgcgtggtgcggttcctgcggcgtggcggcggtgacctcctgccccaaggcctggtgagggcgacggcgtgaggtcggcccgatggcggtgacggtggtgtgcgtccagggcccggcatctggtcgggcgggcgcgcgggacggccttggcaggggcgggcactgacgggaggctcccctcggtgctctgcggtgctggtttggtggaggttttagggaggcttgatggaggtctgtggctggccggtacgggctggtggtggtcgagcatctcagggagaaatccttcttccggcctttgccggagctggcgacggcggcgcctgtgggcgtcgcgctctttcctggaagcgtcgtcgatgtatggtgctccacccctcaccccgcggccttggctccggagggaaacctctgatctgcgggatcgggcgatgaaggcgtcttcacgtcttcttcctccttgggggcatcgtcttggagccggctacaacctgagaccggtggatggcggcatcttcgccgcatggaagggcgacatcttcgtcgcgtgggatggcggcatcttcgccgcgtggaagggcgacatcttcgctgcgtggtttgctgaggcggccgtttcaggggcgccgatttctgtttggcaacgataatggcgtcaagaggagcttgggtcgcggctcgggctttggtagtcggatcttcccggcgtgcccgaggtgctctttagggcacggtcggcgcaagtcctgcatatttcccttgtgaggctcgtcgtcaaagtcgaagttgtcggttgttggcgcgtgtggccatctgttggcatgtgccgtctttgctctgtgtagttgtttgcgagggcgacttgttggtggagctctatggtgaagtcggagtcgcccgttcggagataaccggtgatgacgatgacgcttgcgactccccggcgttcttctttgcagctcatgtttcatgtcggtggacaggttggccggtggtgcccatgttatatgggttgggttgtattggttttagcccggttttccgtcaattaaccgggcaattcttatttcttcttaatcaatgaaaatggcaagtcttttgcctcgtttcaaaaaaaaaaataggagtaactgagctgcagttatcgtcgcccgcgagtggtacacatcaattgacataaagagctacgtcaacggagaattactccaaaatctccagcccaatcgacagagacgattcgttaccttgtcaccgccccttggcgttgcccgacgagtcctctcctgcccaccctgtctcttggttcgcaggaacgattcatcctgaccgcaaacgtccgtccgccggcaaggtcaacaacatcaagcacctcgcacagatcgcgtcgaccaacccgcgcctccttcgttcccatgtgtgcggtccatgatttttttttctcgtttgatgatggacacgaaagcgattactacagaccgaaggcccaataggcccaattaacagcagatggtggattttgttacgggagcagcggccaaatccatgtgcgcgacctgcatagcgaaggaagcccaggcaggattagaggaggatccaatggccagaaacacccactacttgagatccgacggctaaaaacggtaatggcctgagagagctagaagagtgcacggttatataatgtatttaaatttggaacagaaagaaaaaaggtagtaagacgagtgaacggggaagaaaagcagtagaaggcaaacggcgcagctctctctcacgcttctcccgtggtcgacgttgcagtccacacgcgggcgggcacgcggctgggcgcgccggttccaccacctcatctcccgcactccctctgcctcgtatctcgtcgccttcctccacaccccgcaggagcattgccagccgtccgatcgcgc

SEQ ID NO: 37 - skipped

SEQ ID NO: 38 - protein, 783 aa, synthetic construct
  REGION 1..783: note = Arabidopsis thaliana AtMRC
MGFSQAIRLNLASFSSPSPCDYCLTRVVNHKQKSLVAFPSITRRKRHLLLSVQSVLHNTRPNINDNGSAESANVLFDKLFARTHRLERQTNQHSVYPDDDDLPYSNLGVLESDLEAALVALLKREEDLHDAERKLLSDKNKLNRAKEELEKREKTISEASLKHESLQEELKRANVELASQAREIEELKHKLRERDEERAALQSSLTLKEEELEKMRQEIANRSKEVSMAISEFESKSQLLSKANEVVKRQEGEIYALQRALEEKEEELEISKATKKLEQEKLRETEANLKKQTEEWLIAQDEVNKLKEETVKRLGEANETMEDFMKVKKLLTDVRFELISSREALVFSREQMEEKELLLEKQLEELEEQRKSVLSYMQSLRDAHTEVESERVKLRVVEAKNFALEREISVQKELLEDLREELQKEKPLLELAMHDISVIQDELYKKANAFQVSQNLLQEKESSLVEAKLEIQHLKSEQASLELLLQEKDEELAEARNKLGEVNQEVTELKALMISREDQLMEATEMLKEKDVHLHRIEGELGSSKLKVTEAEMVVERIAELTNRLLMSTTNGQNQNAMRINNEISIDSMQQPLEKPHDDYGMENKRLVMELSFTRENLRMKEMEVLAVQRALTFKDEEINVVMGRLEAKEQELKKLKEETINDSEDLKVLYALAQERVGEKTMGDLAIEMLQLEAANLEVEAATSALQKLAKMSTELLTQADMSIEADTTHTVMPERGYSEGSNECLGEVKTEVVRLWSLTEKLLENAGIVAGTSTCMEGVIL

SEQ ID NO: 39 - DNA, 23 bp, synthetic construct
  misc_feature 1..23: note = TaMRC target sequence 1
  source 1..23: mol_type = other DNA
gcggccatgcgcctctccatcgg

SEQ ID NO: 40 - DNA, 23 bp, synthetic construct
  misc_feature 1..23: note = SEQ ID NO: 40: TaMRC target sequence 2
  source 1..23: mol_type = other DNA
caggcagaagctgagtttcatgg

SEQ ID NO: 41 - DNA, 23 bp, synthetic construct
  misc_feature 1..23: note = SEQ ID NO: 41: TaMRC target sequence 3
  source 1..23: mol_type = other DNA
attagatcaaatataactgatgg

SEQ ID NO: 42 - DNA, 23 bp, synthetic construct
  misc_feature 1..23: note = TaMRC target sequence 4
  source 1..23: mol_type = other DNA
aatataactgatggtgataatgg

SEQ ID NO: 43 - DNA, 20 bp, synthetic construct
  misc_feature 1..20: note = TaMRC protospacer sequence 1
  source 1..20: mol_type = other DNA
gcggccatgcgcctctccat

SEQ ID NO: 44 - DNA, 20 bp, synthetic construct
  misc_feature 1..20: note = TaMRC protospacer sequence 2
  source 1..20: mol_type = other DNA
caggcagaagctgagtttca

SEQ ID NO: 45 - DNA, 20 bp, synthetic construct
  misc_feature 1..20: note = TaMRC protospacer sequence 3
  source 1..20: mol_type = other DNA
attagatcaaatataactga

SEQ ID NO: 46 - DNA, 20 bp, synthetic construct
  misc_feature 1..20: note = TaMRC protospacer sequence 4
  source 1..20: mol_type = other DNA
aatataactgatggtgataa

SEQ ID NO: 47 - RNA, 83 nt, synthetic construct
  misc_feature 1..83: note = tracrRNA sequence
  source 1..83: mol_type = other RNA
gttttagagctagaaatagcaagttaaaataaggctagtccgttatcaacttgaaaaagtggcaccgagtcggtgcttttttt

SEQ ID NO: 48 - DNA, 103 bp, synthetic construct
  misc_feature 1..103: note = TaMRC complete sgRNA-encoding nucleic acid sequence 1
  source 1..103: mol_type = other DNA
gcggccatgcgcctctccatgttttagagctagaaatagcaagttaaaataaggctagtccgttatcaacttgaaaaagtggcaccgagtcggtgcttttttt

SEQ ID NO: 49 - DNA, 103 bp, synthetic construct
  misc_feature 1..103: note = TaMRC complete sgRNA-encoding nucleic acid sequence 2
  source 1..103: mol_type = other DNA
caggcagaagctgagtttcagttttagagctagaaatagcaagttaaaataaggctagtccgttatcaacttgaaaaagtggcaccgagtcggtgcttttttt

SEQ ID NO: 50 - DNA, 103 bp, synthetic construct
  misc_feature 1..103: note = TaMRC complete sgRNA-encoding nucleic acid sequence 3
  source 1..103: mol_type = other DNA
attagatcaaatataactgagttttagagctagaaatagcaagttaaaataaggctagtccgttatcaacttgaaaaagtggcaccgagtcggtgcttttttt

SEQ ID NO: 51 - DNA, 103 bp, synthetic construct
  misc_feature 1..103: note = TaMRC complete sgRNA-encoding nucleic acid sequence 4
  source 1..103: mol_type = other DNA
aatataactgatggtgataagttttagagctagaaatagcaagttaaaataaggctagtccgttatcaacttgaaaaagtggcaccgagtcggtgcttttttt

SEQ ID NO: 52 - RNA, 103 nt, synthetic construct
  misc_feature 1..103: note = TaMRC complete sgRNA RNA sequence 1
  source 1..103: mol_type = other RNA
gcggccatgcgcctctccatgttttagagctagaaatagcaagttaaaataaggctagtccgttatcaacttgaaaaagtggcaccgagtcggtgcttttttt

SEQ ID NO: 53 - RNA, 103 nt, synthetic construct
  misc_feature 1..103: note = TaMRC complete sgRNA RNA sequence 2
  source 1..103: mol_type = other RNA
caggcagaagctgagtttcagttttagagctagaaatagcaagttaaaataaggctagtccgttatcaacttgaaaaagtggcaccgagtcggtgcttttttt

SEQ ID NO: 54 - RNA, 103 nt, synthetic construct
  misc_feature 1..103: note = TaMRC complete sgRNA RNA sequence 3
  source 1..103: mol_type = other RNA
attagatcaaatataactgagttttagagctagaaatagcaagttaaaataaggctagtccgttatcaacttgaaaaagtggcaccgagtcggtgcttttttt

SEQ ID NO: 55 - RNA, 103 nt, synthetic construct
  misc_feature 1..103: note = TaMRC complete sgRNA RNA sequence 4
  source 1..103: mol_type = other RNA
aatataactgatggtgataagttttagagctagaaatagcaagttaaaataaggctagtccgttatcaacttgaaaaagtggcaccgagtcggtgcttttttt

SEQ ID NO: 56 - DNA, 4203 bp, synthetic construct
  misc_feature 1..4203: note = Cas9 sequence
  source 1..4203: mol_type = other DNA
atggctcctaagaagaagcggaaggttggtattcacggggtgcctgcggctgacaagaagtactccatcggcctcgacatcggcaccaacagcgtcggctgggcggtgatcaccgacgagtacaaggtcccgtccaagaagttcaaggtcctgggcaacaccgaccgccactccatcaagaagaacctcatcggcgccctcctcttcgactccggcgagacggcggaggcgacccgcctcaagcgcaccgcccgccgccgctacacccgccgcaagaaccgcatctgctacctccaggagatcttctccaacgagatggcgaaggtcgacgactccttcttccaccgcctcgaggagtccttcctcgtggaggaggacaagaagcacgagcgccaccccatcttcggcaacatcgtcgacgaggtcgcctaccacgagaagtaccccactatctaccaccttcgtaagaagcttgttgactctactgataaggctgatcttcgtctcatctaccttgctctcgctcacatgatcaagttccgtggtcacttccttatcgagggtgaccttaaccctgataactccgacgtggacaagctcttcatccagctcgtccagacctacaaccagctcttcgaggagaaccctatcaacgcttccggtgtcgacgctaaggcgatcctttccgctaggctctccaagtccaggcgtctcgagaacctcatcgcccagctccctggtgagaagaagaacggtcttttcggtaacctcatcgctctctccctcggtctgacccctaacttcaagtccaacttcgacctcgctgaggacgctaagcttcagctctccaaggatacctacgacgatgatctcgacaacctcctcgctcagattggagatcagtacgctgatctcttccttgctgctaagaacctctccgatgctatcctcctttcggatatccttagggttaacactgagatcactaaggctcctctttctgcttccatgatcaagcgctacgacgagcaccaccaggacctcaccctcctcaaggctcttgttcgtcagcagctccccgagaagtacaaggagatcttcttcgaccagtccaagaacggctacgccggttacattgacggtggagctagccaggaggagttctacaagttcatcaagccaatccttgagaagatggatggtactgaggagcttctcgttaagcttaaccgtgaggacctccttaggaagcagaggactttcgataacggctctatccctcaccagatccaccttggtgagcttcacgccatccttcgtaggcaggaggacttctaccctttcctcaaggacaaccgtgagaagatcgagaagatccttactttccgtattccttactacgttggtcctcttgctcgtggtaactcccgtttcgcttggatgactaggaagtccgaggagactatcaccccttggaacttcgaggaggttgttgacaagggtgcttccgcccagtccttcatcgagcgcatgaccaacttcgacaagaacctccccaacgagaaggtcctccccaagcactccctcctctacgagtacttcacggtctacaacgagctcaccaaggtcaagtacgtcaccgagggtatgcgcaagcctgccttcctctccggcgagcagaagaaggctatcgttgacctcctcttcaagaccaaccgcaaggtcaccgtcaagcagctcaaggaggactacttcaagaagatcgagtgcttcgactccgtcgagatcagcggcgttgaggaccgtttcaacgcttctctcggtacctaccacgatctcctcaagatcatcaaggacaaggacttcctcgacaacgaggagaacgaggacatcctcgaggacatcgtcctcactcttactctcttcgaggatagggagatgatcgaggagaggctcaagacttacgctcatctcttcgatgacaaggttatgaagcagctcaagcgtcgccgttacaccggttggggtaggctctcccgcaagctcatcaacggtatcagggataagcagagcggcaagactatcctcgacttcctcaagtctgatggtttcgctaacaggaacttcatgcagctcatccacgatgactctcttaccttcaaggaggatattcagaaggctcaggtgtccggtcagggcgactctctccacgagcacattgctaaccttgctggttcccctgctatcaagaagggcatccttcagactgttaaggttgtcgatgagcttgtcaaggttatgggtcgtcacaagcctgagaacatcgtcatcgagatggctcgtgagaaccagactacccagaagggtcagaagaactcgagggagcgcatgaagaggattgaggagggtatcaaggagcttggttctcagatccttaaggagcaccctgtcgagaacacccagctccagaacgagaagctctacctctactacctccagaacggtagggatatgtacgttgaccaggagctcgacatcaacaggctttctgactacgacgtcgaccacattgttcctcagtctttccttaaggatgactccatcgacaacaaggtcctcacgaggtccgacaagaacaggggtaagtcggacaacgtcccttccgaggaggttgtcaagaagatgaagaactactggaggcagcttctcaacgctaagctcattacccagaggaagttcgacaacctcacgaaggctgagaggggtggcctttccgagcttgacaaggctggtttcatcaagaggcagcttgttgagacgaggcagattaccaagcacgttgctcagatcctcgattctaggatgaacaccaagtacgacgagaacgacaagctcatccgcgaggtcaaggtgatcaccctcaagtccaagctcgtctccgacttccgcaaggacttccagttctacaaggtccgcgagatcaacaactaccaccacgctcacgatgcttaccttaacgctgtcgttggtaccgctcttatcaagaagtaccctaagcttgagtccgagttcgtctacggtgactacaaggtctacgacgttcgtaagatgatcgccaagtccgagcaggagatcggcaaggccaccgccaagtacttcttctactccaacatcatgaacttcttcaagaccgagatcaccctcgccaacggcgagatccgcaagcgccctcttatcgagacgaacggtgagactggtgagatcgtttgggacaagggtcgcgacttcgctactgttcgcaaggtcctttctatgcctcaggttaacatcgtcaagaagaccgaggtccagaccggtggcttctccaaggagtctatccttccaaagagaaactcggacaagctcatcgctaggaagaaggattgggaccctaagaagtacggtggtttcgactcccctactgtcgcctactccgtcctcgtggtcgccaaggtggagaagggtaagtcgaagaagctcaagtccgtcaaggagctcctcggcatcaccatcatggagcgctcctccttcgagaagaacccgatcgacttcctcgaggccaagggctacaaggaggtcaagaaggacctcatcatcaagctccccaagtactctcttttcgagctcgagaacggtcgtaagaggatgctggcttccgctggtgagctccagaagggtaacgagcttgctcttccttccaagtacgtgaacttcctctacctcgcctcccactacgagaagctcaagggttcccctgaggataacgagcagaagcagctcttcgtggagcagcacaagcactacctcgacgagatcatcgagcagatctccgagttctccaagcgcgtcatcctcgctgacgctaacctcgacaaggtcctctccgcctacaacaagcaccgcgacaagcccatccgcgagcaggccgagaacatcatccacctcttcacgctcacgaacctcggcgcccctgctgctttcaagtacttcgacaccaccatcgacaggaagcgttacacgtccaccaaggaggttctcgacgctactctcatccaccagtccatcaccggtctttacgagactcgtatcgacctttcccagcttggtggtgataagcgtcctgctgccaccaaaaaggccggacaggctaagaaaaagaagtag

SEQ ID NO: 57 - DNA, 561 bp, synthetic construct
  misc_feature 1..561: note = Cys 4 endoribonuclease nucleic acid sequence
  source 1..561: mol_type = other DNA
atggaccactacctcgacatcaggctcaggccagacccagagttcccaccagcccagctcatgtccgtcctcttcggcaagctccaccaggccctcgtggcccagggcggcgacaggatcggcgtgtccttcccagacctcgacgagtccaggtccaggctcggcgagaggctccgcatccacgcctccgccgacgacctcagggccctcctcgccaggccgtggctggagggcctcagggaccacctccagttcggcgagccagccgtggtgccacacccaaccccatacaggcaagtgtccagggtgcaagccaagtccaacccagagaggctcaggaggaggctcatgaggaggcacgacctctccgaggaagaggccaggaagcgcatcccagacaccgtggccagggccctcgacctcccattcgtgaccctcaggtcccagtccaccggccagcacttccgcctcttcatcaggcacggcccactccaggtgaccgccgaggagggcggctttacctgctacggcctctccaagggcggcttcgtgccgtggttc

SEQ ID NO: 58 - DNA, 363 bp, synthetic construct
  misc_feature 1..363: note = Wheat U6 promoter
  source 1..363: mol_type = other DNA
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttg

SEQ ID NO: 59 - DNA, 982 bp, synthetic construct
  misc_feature 1..982: note = Maize Ubiquitin1 promoter
  source 1..982: mol_type = other DNA
tgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaag

SEQ ID NO: 60 - protein, 770 aa, synthetic construct
  REGION 1..770: note = Solanum MRC StMRC (PGSC0003DMT400010993)
MALPALPRATLSFSSLCQPTEFCFMRLEWKKRLVLMTAHHGRGPSSRIVRSVLDNRKSNITGDEATEPARVLLERLFAQTQKLEQQIGRNIYFPQVAELGLNLGKLESDLQDALAALKKKEEDIQDTERKVLMEYNELNRAKIELEQRVEEMAAANSRQEKLENELRQANLILVSQAAEIEDLKFRFNEIDQEISAAQTALVSKEDEINKMMIELKNKSDEVANTESQLRTKGELLDTANEVVQRQEVELQNLQREIQEKEKELQVFLTMQKTEEEKLKVSKSNLEKQAMDWLIAKQEMKKLEVETSNYGGEANRSLEDFRRVKKLLADVRSELVSSQRALTSSRKKMEEQENLLEDRLEELEEQRRSVMSYMTSLKEAQNEVENEKVQLTVAEARNKELERDLSIEKELVEELQTENNIKKSSLHVAINEKSALQEELDCKSAEFGETQNLLQVKESELVDARLEIQHLKSQCASLQLMLEEKDKELLDSRKTVDELNQEIAELRVNMNSQEQQLIQATSMLKEKEESMQIMQLELNDTKMKYSEAETVVEHMVDLTNKLVISVKDDVLSPLSHTDEMWSSQLVEKPTDAFRWHKNQLENELELTRESLRSREMDSLAAQRALKLKEQELKIVRQKLNDREEEINKMKNMTRDADGPRQSYVLAQERTGEKSTGDLAVEKLQFEGAQLEVEAATTALQKLAELSRDLLNKASLTIEADYDSSLLLVDIPETAANVSSSFECLAEVYSEMAQLSALSEKLVKEAGILCPQ

SEQ ID NO: 61 - DNA, 3877 bp, synthetic construct
  misc_feature 1..3877: note = Solanum StMRC (PGSC0003DMG400004306) genomic
  source 1..3877: mol_type = other DNA
atggcgttgccagccttaccgcgcgccactttatcgttttcctctctttgccagccaacggaggtgaaagtctcttcttcttttttgttgttgttttctccttatttattctgttcacagagtatttttttttacattttcctacatttcaatttcttcaatgggatcttttgctttttctgcaaaaataaataaataaattgttaatgttggaaaagattaggtatatagatgtctttacaaccttcctttatagagtaaattgcacaggtttgctaatggtttaggtaacaattcaaagaaggaacttacgagttctgccgctagcactgatttacaactctaaattgtttccgattgctcttttatttcatattactattagtagtgataagagatggatgtcgtttataactagatacatgatcatacgtaatataggtggttattttgtttcattcaatcaaatattaaccagattggaattgttgtgaaaatctgtaaagtaaaataaggaattaaggattggaacaattaatgtttaatgtttgagttgagaagagtaaaattttagtactaatatctgaatttagagagagattgcatagtagacaaatttggtcatggagatacataatagtgcttcaacatccataattgtgtccaagcacttttgttcttctcagcatttgtcatgttggcaaaaatatttttgcttcttaaaatggttcggagtgatgttaactccaaagaacagatctcagttctcggacatctctctaattaggatgtagctgatatggtcatcaactgtgtagataggattctatttggttattacaactttataattttctttggaggttgaaataaggttcacctttttggaatgtgaaagtatacaattgcatttatgttgatggatcttgcatgaaagttgtgatataaatgttccataatgatttatttcagtgagtttggatgaaatcgatcgatcaatcaatcaaccatacctcaatcccaaactagttggtcaaatatatgaattttctatatccattctactttatccgtgaccatttgttgtatgagtgaacgttaaaatatagtctgaacaaggaagtaacaaattgttatgaccatgggatgtgagaaaccataacaagagtatacaagacatctcttattcttatttcttcaccaagagttgtggggatggatggggaggtgaaaaatgaaatatatcaacggtttcccctgttgatttatttcaccttgtcttacaaaaggaaaaataagggaagctaaagaagaaaaattaagagattaactatgagccgctttcttgcatatatactggagaaaatacactgtactgactactgtggagtaaaactaattcagttatcttgcccgtactaccaaattgaacttgtgcataggaattcttctccaattgtgcctttgtcttatacatcttcacttgcttatcactggtctgttaatactactgtatgaattgtgctgggagtcccatggaatagatgacaggtttgtctttcataaaagtaggaagctctacacgtgataatattgtggtgtaattactgacatctctttgtgtacagttctgttttatgaggctcgaatggaagaagagattagtgcttatgacagctcatcatgggcggggtccttcctcaagaattgtcaggtctgtcttggataacaggaaatcaaatatcaccggcgatgaagcaactgagccggctagggttcttcttgagaggttgtttgcccagacccagaaactagaacaacagattggcagaaatatttattttcctcaggttgctgagctgggactaaatcttggcaagctagagtcggatttgcaggatgctcttgcagccttgaagaaaaaggaagaagatattcaagatacagagagaaaagtattgatggagtacaatgaattaaaccgtgcaaagatagaattggagcaacgtgtggaggagatggcagctgctaattctaggcaggaaaaactggaaaatgagctaaggcaggctaatctgatcttagtatctcaagctgcagaaattgaagatctaaagtttcgtttcaacgagatagatcaggagatatctgctgcgcaaacagccctagtttcaaaagaagatgaaataaataaaatgatgattgagttgaagaataaaagtgatgaagtggctaatactgaatcacaactcagaaccaagggtgaactactcgatacagcaaatgaagtagttcaaagacaggaggttgaactacaaaatctccaaagagaaattcaagagaaagagaaagagctacaagtcttcttgacgatgcagaaaaccgaagaagagaaacttaaagtttccaaatccaatttggagaagcaggcaatggattggctcatagcaaagcaagaaatgaagaaattggaagtggaaacatctaactatggtggagaagcaaatcggtcccttgaggatttcagaagagtcaagaagctacttgccgatgtaaggtctgagttagtctcatctcagagagctttgacatcctctagaaagaaaatggaagagcaggaaaatctattagaagatcgtctcgaagaacttgaagagcagagaagaagtgttatgtcttacatgacaagtttgaaagaagctcaaaatgaggtagagaatgagaaagtgcaacttacggttgctgaagctcgaaacaaagaacttgagagggatttatccatagaaaaggagctcgttgaggagttgcagactgagaataatattaagaaatcttctctgcatgtagctatcaatgaaaaatctgctctccaggaggagcttgactgtaagagtgcagagtttggagaaacacagaatcttcttcaggttaaagagtcagagctagtagatgctagattagagattcagcacttgaagtctcagtgcgcttctcttcagctgatgttggaagaaaaagataaggaacttctggattcaagaaagacagtagatgaactaaatcaggaaatagctgagctgagggtgaacatgaacagtcaagaacagcaacttattcaggcaacaagtatgttgaaagaaaaagaggaatccatgcagataatgcaacttgagttaaatgatacaaaaatgaaatattcagaagctgagaccgttgtggaacatatggtagacctgactaacaaattggttatttctgttaaggatgacgtgttgagcccactcagtcacacagatgaaatgtggtcatctcagctggtggagaaaccaactgatgcttttaggtggcacaaaaaccagcttgaaaatgaacttgagttaaccagagaaagcctgaggagtagagaaatggattctcttgcagcacaaagggctcttaaactcaaagagcaggagctcaaaatagttcgtcaaaaattaaatgatagggaggaagaaataaataaaatgaagaatatgacccgggacgcagatggcccaaggcaatcttatgttttggcacaggaaagaacaggtgaaaagagcactggagatctggcagttgaaaagctccaattcgagggagctcaattggaagttgaagctgcaaccactgctctccagaactcgctgaactcagccgtgaccttttgaataaagctagtttgaccattgaggctgactatgatagcagccttttgttggttgacatcccagaaactgcagcaaatgtctctagcagttttgagtgtcttgctgaagtttattcagagatggcacaactttcagctttgagtgagaagctagtgaaagaagctggtattttatgcccccagtag

SEQ ID NO: 62 - DNA, 2313 bp, synthetic construct
  misc_feature 1..2313: note = Solanum MRC CDS StMRC (PGSC0003DMT400010993)
  source 1..2313: mol_type = other DNA
atggcgttgccagccttaccgcgcgccactttatcgttttcctctctttgccagccaacggagttctgttttatgaggctcgaatggaagaagagattagtgcttatgacagctcatcatgggcggggtccttcctcaagaattgtcaggtctgtcttggataacaggaaatcaaatatcaccggcgatgaagcaactgagccggctagggttcttcttgagaggttgtttgcccagacccagaaactagaacaacagattggcagaaatatttattttcctcaggttgctgagctgggactaaatcttggcaagctagagtcggatttgcaggatgctcttgcagccttgaagaaaaaggaagaagatattcaagatacagagagaaaagtattgatggagtacaatgaattaaaccgtgcaaagatagaattggagcaacgtgtggaggagatggcagctgctaattctaggcaggaaaaactggaaaatgagctaaggcaggctaatctgatcttagtatctcaagctgcagaaattgaagatctaaagtttcgtttcaacgagatagatcaggagatatctgctgcgcaaacagccctagtttcaaaagaagatgaaataaataaaatgatgattgagttgaagaataaaagtgatgaagtggctaatactgaatcacaactcagaaccaagggtgaactactcgatacagcaaatgaagtagttcaaagacaggaggttgaactacaaaatctccaaagagaaattcaagagaaagagaaagagctacaagtcttcttgacgatgcagaaaaccgaagaagagaaacttaaagtttccaaatccaatttggagaagcaggcaatggattggctcatagcaaagcaagaaatgaagaaattggaagtggaaacatctaactatggtggagaagcaaatcggtcccttgaggatttcagaagagtcaagaagctacttgccgatgtaaggtctgagttagtctcatctcagagagctttgacatcctctagaaagaaaatggaagagcaggaaaatctattagaagatcgtctcgaagaacttgaagagcagagaagaagtgttatgtcttacatgacaagtttgaaagaagctcaaaatgaggtagagaatgagaaagtgcaacttacggttgctgaagctcgaaacaaagaacttgagagggatttatccatagaaaaggagctcgttgaggagttgcagactgagaataatattaagaaatcttctctgcatgtagctatcaatgaaaaatctgctctccaggaggagcttgactgtaagagtgcagagtttggagaaacacagaatcttcttcaggttaaagagtcagagctagtagatgctagattagagattcagcacttgaagtctcagtgcgcttctcttcagctgatgttggaagaaaaagataaggaacttctggattcaagaaagacagtagatgaactaaatcaggaaatagctgagctgagggtgaacatgaacagtcaagaacagcaacttattcaggcaacaagtatgttgaaagaaaaagaggaatccatgcagataatgcaacttgagttaaatgatacaaaaatgaaatattcagaagctgagaccgttgtggaacatatggtagacctgactaacaaattggttatttctgttaaggatgacgtgttgagcccactcagtcacacagatgaaatgtggtcatctcagctggtggagaaaccaactgatgcttttaggtggcacaaaaaccagcttgaaaatgaacttgagttaaccagagaaagcctgaggagtagagaaatggattctcttgcagcacaaagggctcttaaactcaaagagcaggagctcaaaatagttcgtcaaaaattaaatgatagggaggaagaaataaataaaatgaagaatatgacccgggacgcagatggcccaaggcaatcttatgttttggcacaggaaagaacaggtgaaaagagcactggagatctggcagttgaaaagctccaattcgagggagctcaattggaagttgaagctgcaaccactgctctccagaaactcgctgaactcagccgtgaccttttgaataaagctagtttgaccattgaggctgactatgatagcagccttttgttggttgacatcccagaaactgcagcaaatgtctctagcagttttgagtgtcttgctgaagtttattcagagatggcacaactttcagctttgagtgagaagctagtgaaagaagctggtattttatgcccccagtag

SEQ ID NO: 63 - protein, 494 aa, unidentified
  REGION 1..494: note = Selaginella Species
QVSAVLDTPSERTNSSDPSEPARVALERLFEELKRKEEELKTAETVAREDHHQLENSKFALVKRGDVLEDARQGQGKKRDEIGNLYKELAEQAKEAENAQKVVDEQQRQLEALQKTLSKKQELIARSKAQIFAKDEALAQCRRELKVRDSRLKHANEVIAQQAVELKELRSSLELKEAGLRTLEENVKFKQEQIQKTEADLSTRVIAYLSVERELKSLEADLSKSKALNVEAGKELKGVKDLLTEVQEELRFSQMRLQQYKKTVEEQSIQIKSQQEEIALQKSLVESFEFSLAEAKERTKREEEQVRLAKGSYKKLEEQSTNERLEAEKLQLELRNEKSALQEATTEINSLKRELQQKETALSDTQLALRLKEAELTASQVELQELKSDFTSMKLELDQKDSELRHAQTVVNALQQDVKRLKALLSAKEEKHVEVVAALKDKEEELVSMRKKLDANNVKMSQVNFAVEQISALSEALVDSIADDKPSLLSRESV

SEQ ID NO: 64 - protein, 496 aa, Brassica napus
TVKSVLNNTRPSFNDNGADEPSKILLDKLFARMALLKREEDLQDAERKVLSEKKKLSKAKEGLEKRERVILQASLKHESLQEELKRANVELASQAREIEELKHKLRERDEELVAMQASLTFKERELDRMRVEISIKTKEASVASFEFENKSQLLIQANEIVKKQEDEIEALQRALKEKEEELEVSTEAKKLEQEKLRETEASLRKQTEEWLVAQEEVSKLQEETVKRLGEANETMEDFRRVRKLLTDVRFELVSSREVLLSSREQMGEKEVLLEKQLEELEEQRRSVLSYMQSLRDARGEVESERVKLRVAEAKNFALEREISIQKELLEELREELKKEKSLLEQAMHDVSTIQDELDKKTNEFQVSQTLLQEKESSLVEAKLEIQHLKSEQASLELLLQEKDEELTEARNQLEVVNREVTELKMLMRTREDQLTQATELLKEKDVHLNRIEDELGSSKIQASEAEMVVERIAELTSRLVQDQMQQQPLEKQPYGD

SEQ ID NO: 65 - protein, 496 aa, unidentified
  REGION 1..496: note = Amborella trichopoda
LIRSVVDKPTSNIGSNGPSESARVLLERLFAQAALKKKEEDLQYAEKMVLMDRAKLTDVKQDLDHREEEIIAAHAKQAKLEEDLKKAQEDFASQEKQIEELKHLVKEKDREMQNARSALSLKEAGLDILRNELVEKTEEVERINLDLKSRDQILEQTNKVIRKQEAEVQDLKEAIMSKEEELADTIQQRKNDEEKLKNAKANLEQRAVEWLSSQEELKKIAEEASKYKAEAKGTAQELKRVRVLLADVKTELIASQKSLASSGRRLDDQGVELKKQLEDLNEEKSLLTSYMTNLKEAQLEVESERKKLRYAEARNQELEQKISKEQAMIEKLQNELNREKLSLEKTTKDVGSLTALLEQKISDLDNTLKLLKVKESELVSARSEIQLLKSDHESIQLLLKEKDVELSLANKNLEDLDKEVRDLKKLMREKEEQLIQATLGLQEKDQLVEMMRLEIDDTKLKYLEAAAVVGRIMQLTNILVNTAREETWNLESHGSL

SEQ ID NO: 66 - protein, 496 aa, unidentified
  REGION 1..496: note = Gossypium raimondii
IVESVLNNSKSSINDNGAAESAKVLLERLFAQTALKQKEDDLQDAEKMVVLEQSELSRAKDELEQREKEIAAASSKHEKLEEKLTQANLAFASQASQIEDLKLQLKEQDHKVAAAQSTLSAKEDEMDKMRHELVKKTEEAEKIRSELTSKSQLLNEANEVMKKQEIELQELREAIWEREEELETSLTQRKLEEEKLKVAEAKLQQQTMEWLLAQEELKKLAEQASRHMGEANEAFKDFTRVKQLLSDVRSELVSSQKSLASSRQQMEQQEQLLKMQLEELEEQRKSVASYMESLKNAQIEVESERVKLRVVEARNKDLERDLSVERELIKELQEELKKEKYSLQLAIQDASFLRKQLGKKHTEFVEMNNVLQNKEVDLVEAKLEIQHLKSERASLQLILEEKDQELSDAKKNLEQLNQEIAELKMLMSSKENQLIQATALLKEKDEYALKVQDELNDTKMKFSEAETVIERIAELTNRLVISVKDEDNNVLRPVDD

SEQ ID NO: 67 - protein, 496 aa, Vitis vinifera
IVKSVLNNRKSSINDNGSTEPARVLLERLFAQVALKKKEEDLQDAAGMVLMEHTELNRAKEELKRHAEEIAVACSKHEKLEEELKQANLNLASRARQIEDLKLQLKDRDQEIFAARSALSSKQDEMDKMRNELMKKTEEAAKKESELQSMAKLLDEANEVVKKQEIELQELQKSIQEKEEELEESMMLRKLEEKKLKVAEANLEKKTMDWLLAKEELKKLAEDAAKHMGESNKTMKEFRRAKRLLHDVRSELVSSQKSLASSRQKMQEQEKLLEKQLAELEEQKTSINHYMTSLKDAQIEVESERVKLRVAESRNKELEWDLSVKKELMEELQEELRKEKSSLQQVIQETSFLQKELDQKTTEFGELHNLLQVKESELVEARLEIQHLKSEQVSLQLILKERDLELFNAQKKLEEVNQEVSELKMLMNNREDQLMQATTLLKEKEEHLLIMQHELNDTKLKFSEAESVVERIVDLTNKLVICTKDEECTATSPFDD

SEQ ID NO: 68 - protein, 496 aa, Manihot esculenta
IVKSVLNCSKSSINDNGATEPARILLERLFAQDALRKKEEDLQHAEKQVLSEHNDLNFAKQELQQREKEIAVAHSKHEKLEGELREANLNLASQARQIEDIKLQLKEREEEVAAGQSALLIKQHEIEKKISELTKKSEEVAKMDSELQYKAQLLDEANEVVKKQEIEIQRLKKGLQEKEKELEVSVALRKVEEEKLKVVQTNLEKQAMEWLIAQEGLKRLANETSKRMVETNETMKDFKRVKKLLVDVRSELVSSQKSLAFSRKRMEEQDLLLKKQLLELEEERESVMSYMTSLKDAQMEVESEKGKLRAAEARNKELEQKLSLEKEIMEEIREELNKEKSSLEQMVQEMSYLQQELAAKNTEFGEMHDILQFKESELVEAKLEIQHLKSVQCSLQLLSEEKDLQLLDAKKKLEELDQEVAELKMLLSSKEDQLIQATNMLMEKEERVQMMQDELNDARLKISEAETVVERIVDLTNKLVITVKDEDYNAVRPSGS

SEQ ID NO: 69 - protein, 495 aa, Medicago truncatula
TVKSVLNDNRPSFNNYGAPESARLLERLFEQMELKEKEDHLQEVERTVLLENGKLKDAKEELERQEGEIKAAREKYERLEDEMKEAMASLVSQAGQVEELKLRLRDRDSETDGLRDALSLKEEEMEKMKIGLAKKSEEAAYVDSELRQKVQLLSEANEVVKKQEIELQELRSVVQQREEELRLSVAARDVEGEKLKVAEASLEKQAMEWLLTQEELKRLEEEASKHAQERSETLEDFRRVKKLLSDVRSELVSSQQSLASSRYKMQVQEGLLEQQLAELADQRESVMLYMENLKDAQIEVENERTKLSVAEALNKELEQDLSVEKELMKKLQEELKKEKASLEQAVQEMALLQEELDIKSAEFKEKSALLDVKESELVDAKLQIQELKTEKASLQALLEEKDLELSSARKMLVELNQEISDLKMLMNDKETQLIEATNMLREKDEHVKVIQNKLNNTSLKAFEAETVVGRVLDLTNKLVASIKNEDINSSRPLNE

SEQ ID NO: 70 - protein, 496 aa, unidentified
  REGION 1..496: note = Citrus sinensis
LVRAVLDGKKSSVNGYGLGEPARILLERLFAQAALKKKEEDLEDAERRVCLEHSELNRAKEELLRREREIDVACSRHEKLEEELGQSNLKLVSQARHIEDLKLRLKERDQEIAAMQSALSLKELELEKMRSELLKKSEEAAKIDSELKSKAQMLNEANEVVKKQETEIQSLRKVIQEKEEELEASVALRKVEEEKLKVVEANLEKRTMEWLLSQDALKKLAEEASRRMEETNDTLEDFRRVKKLLSDVRSELVSSQKSLASSRKQMEEQEHLLGKQLVELEEQKKSLTSYMTSLKDAQVEVESERVKLRVTEARNKELERDLSMEKELVEELQNELNKEKYSLQQAIDEVSSLQEELGRKNTEFGETENLLRVKESDLVEAKLEIQNLKSKQASLQLILEEKDFELSNARQMLEELNNEVRELKMIMSSREEQLVQAMDTLQEKDEHVLILQNELDGTKLKVSEAETVVEQIVDLTHKLVISNKNDESSTSMPTDD

SEQ ID NO: 71 - protein, 496 aa, Solanum tuberosum
IVRSVLDNRKSNITGDEATEPARVLLERLFAQAALKKKEEDIQDTERKVLMEYNELNRAKIELEQRVEEMAAANSRQEKLENELRQANLILVSQAAEIEDLKFRFNEIDQEISAAQTALVSKEDEINKMMIELKNKSDEVANTESQLRTKGELLDTANEVVQRQEVELQNLQREIQEKEKELQVFLTMQKTEEEKLKVSKSNLEKQAMDWLIAKQEMKKLEVETSNYGGEANRSLEDFRRVKKLLADVRSELVSSQRALTSSRKKMEEQENLLEDRLEELEEQRRSVMSYMTSLKEAQNEVENEKVQLTVAEARNKELERDLSIEKELVEELQTENNIKKSSLHVAINEKSALQEELDCKSAEFGETQNLLQVKESELVDARLEIQHLKSQCASLQLMLEEKDKELLDSRKTVDELNQEIAELRVNMNSQEQQLIQATSMLKEKEESMQIMQLELNDTKMKYSEAETVVEHMVDLTNKLVISVKDDVLSPLSHTDE

SEQ ID NO: 72 - protein, 496 aa, Solanum lycopersicum
IVRSVLDNRKSNITGEEETEPARVLLERLFAQAALKKKEDDIQDTERKVLMEYNELNRAKIELEQRVEEMEAANSRQEKLENELRQANLVLVSQAAEIEDLKFRFNEIDQEISAAQIALVSKEDEINKMMIELKNKCDEAAKTESQLRTKGELLDTANEVVQRQEVELQNLRREIQEKEKELQVFLTMQKTEDEKLKVSKSNLEKQAMDWLIAKQEMKKLEEETSKYGGGANRSLEDFRRVKKLLADVRSELVSSQRALTSSRKKMEEQENLLENRLEELEEQRKSVMSYMTSLKEAQNEVENEKMQLTVAEARNKELERDLSMEKELVEELQTENNIKKSSLYVAINEKSALQEELDRKSAEFGETQNLLQVTESELVDARLEIQHLKSQCASLQLMLEEKNKELLDSRKTLDELNQEIAELRVLMNSQEQQLIQATSMLKEKEEFMQIMQLELNDTKKKYLEAETVVEQMVDLTNKLVISVKDDVLSSLSHTDE

SEQ ID NO: 73 - DNA, 20 bp, synthetic construct
  misc_feature 1..20: note = Kompetitive Allele Specific PCR (KASP) Marker
  source 1..20: mol_type = other DNA
agcaacagttagggagctgc

SEQ ID NO: 74 - DNA, 20 bp, synthetic construct
  misc_feature 1..20: note = Kompetitive Allele Specific PCR (KASP) Marker
  source 1..20: mol_type = other DNA
agcaacagttagggagctgt

SEQ ID NO: 75 - DNA, 22 bp, synthetic construct
  misc_feature 1..22: note = Kompetitive Allele Specific PCR (KASP) Marker
  source 1..22: mol_type = other DNA
cctcgattcatttgatctggcg

SEQ ID NO: 76 - DNA, 20 bp, synthetic construct
  misc_feature 1..20: note = Kompetitive Allele Specific PCR (KASP)
  source 1..20: mol_type = other DNA
catacggctcagatgctcgc

SEQ ID NO: 77 - DNA, 20 bp, synthetic construct
  misc_feature 1..20: note = Kompetitive Allele Specific PCR (KASP) Marker
  source 1..20: mol_type = other DNA
catacggctcagatgctcgt

SEQ ID NO: 78 - DNA, 18 bp, synthetic construct
  misc_feature 1..18: note = Kompetitive Allele Specific PCR (KASP) Marker
  source 1..18: mol_type = other DNA
gcaagatcgccagtgagc

SEQ ID NO: 79 - DNA, 22 bp, synthetic construct
  misc_feature 1..22: note = Kompetitive Allele Specific PCR (KASP) Marker
  source 1..22: mol_type = other DNA
attcaagggaagacacaggagc

SEQ ID NO: 80 - DNA, 22 bp, synthetic construct
  misc_feature 1..22: note = Kompetitive Allele Specific PCR (KASP) Marker
  source 1..22: mol_type = other DNA
attcaagggaagacacaggagt

SEQ ID NO: 81 - DNA, 21 bp, synthetic construct
  misc_feature 1..21: note = Kompetitive Allele Specific PCR (KASP) Marker
  source 1..21: mol_type = other DNA
tcatgacaacgagactgtgca

SEQ ID NO: 82 - DNA, 22 bp, synthetic construct
  misc_feature 1..22: note = Kompetitive Allele Specific PCR (KASP) Marker
  source 1..22: mol_type = other DNA
cttgatctttggcatcaagtgc

SEQ ID NO: 83 - DNA, 22 bp, synthetic construct
  misc_feature 1..22: note = Kompetitive Allele Specific PCR (KASP) Marker
  source 1..22: mol_type = other DNA
cttgatctttggcatcaagtgt

SEQ ID NO: 84 - DNA, 21 bp, synthetic construct
  misc_feature 1..21: note = Kompetitive Allele Specific PCR (KASP) Marker
  source 1..21: mol_type = other DNA
acagccctcgagtccaattta

SEQ ID NO: 85 - DNA, 20 bp, synthetic construct
  misc_feature 1..20: note = Kompetitive Allele Specific PCR (KASP) Marker
  source 1..20: mol_type = other DNA
tgtaggagagatggagctgc

SEQ ID NO: 86 - DNA, 20 bp, synthetic construct
  misc_feature 1..20: note = Kompetitive Allele Specific PCR (KASP) Marker
  source 1..20: mol_type = other DNA
tgtaggagagatggagctgt

SEQ ID NO: 87 - DNA, 18 bp, synthetic construct
  misc_feature 1..18: note = Kompetitive Allele Specific PCR (KASP) Marker
  source 1..18: mol_type = other DNA
gcttcgacctccgcagca

SEQ ID NO: 88 - DNA, 24 bp, synthetic construct
  misc_feature 1..24: note = Kompetitive Allele Specific PCR (KASP) Marker
  source 1..24: mol_type = other DNA
ctcgttgtcatgaacttgaatcac

SEQ ID NO: 89 - DNA, 24 bp, synthetic construct
  misc_feature 1..24: note = Kompetitive Allele Specific PCR (KASP) Marker
  source 1..24: mol_type = other DNA
ctcgttgtcatgaacttgaatcat

SEQ ID NO: 90 - DNA, 20 bp, synthetic construct
  misc_feature 1..20: note = Kompetitive Allele Specific PCR (KASP) Marker
  source 1..20: mol_type = other DNA
tcgaccttctccctttcctg

SEQ ID NO: 91 - DNA, 23 bp, synthetic construct
  misc_feature 1..23: note = Kompetitive Allele Specific PCR (KASP) Marker
  source 1..23: mol_type = other DNA
ttgagaagcagagtttaggatgg

SEQ ID NO: 92 - DNA, 23 bp, synthetic construct
  misc_feature 1..23: note = Kompetitive Allele Specific PCR (KASP) Marker
  source 1..23: mol_type = other DNA
ttgagaagcagagtttaggatga

SEQ ID NO: 93 - DNA, 25 bp, synthetic construct
  misc_feature 1..25: note = Kompetitive Allele Specific PCR (KASP) Marker
  source 1..25: mol_type = other DNA
acgtttgaagtcagtgataatacca

SEQ ID NO: 94 - DNA, 21 bp, synthetic construct
  misc_feature 1..21: note = Kompetitive Allele Specific PCR (KASP) Marker
  source 1..21: mol_type = other DNA
acttggttgcatcagagagtc

SEQ ID NO: 95 - DNA, 21 bp, synthetic construct
  misc_feature 1..21: note = Kompetitive Allele Specific PCR (KASP) Marker
  source 1..21: mol_type = other DNA
acttggttgcatcagagagtt

SEQ ID NO: 96 - DNA, 25 bp, synthetic construct
  misc_feature 1..25: note = Kompetitive Allele Specific PCR (KASP) Marker
  source 1..25: mol_type = other DNA
acttccattttaggatcgcatttaa

SEQ ID NO: 97 - DNA, 22 bp, synthetic construct
  misc_feature 1..22: note = Kompetitive Allele Specific PCR (KASP) Marker
  source 1..22: mol_type = other DNA
cgagttcctgtacttgttcctg

SEQ ID NO: 98 - DNA, 22 bp, synthetic construct
  misc_feature 1..22: note = Kompetitive Allele Specific PCR (KASP) Marker
  source 1..22: mol_type = other DNA
cgagttcctgtacttgttccta

SEQ ID NO: 99 - DNA, 21 bp, synthetic construct
  misc_feature 1..21: note = Kompetitive Allele Specific PCR (KASP) Marker
  source 1..21: mol_type = other DNA
gatgctgtacgctctgaattg

SEQ ID NO: 100 - DNA, 24 bp, synthetic construct
  misc_feature 1..24: note = Kompetitive Allele Specific PCR (KASP) Marker
  source 1..24: mol_type = other DNA
tggctgttcaagaaaaggattcag

SEQ ID NO: 101 - DNA, 24 bp, synthetic construct
  misc_feature 1..24: note = Kompetitive Allele Specific PCR (KASP) Marker
  source 1..24: mol_type = other DNA
tggctgttcaagaaaaggattcaa

SEQ ID NO: 102 - DNA, 22 bp, synthetic construct
  misc_feature 1..22: note = Kompetitive Allele Specific PCR (KASP) Marker
  source 1..22: mol_type = other DNA
tgaagctcagcaatttcactgc

SEQ ID NO: 103 - DNA, 20 bp, synthetic construct
  misc_feature 1..20: note = Kompetitive Allele Specific PCR (KASP) Marker
  source 1..20: mol_type = other DNA
agcaacagttagggagctgc

SEQ ID NO: 104 - DNA, 20 bp, synthetic construct
  misc_feature 1..20: note = Kompetitive Allele Specific PCR (KASP) Marker
  source 1..20: mol_type = other DNA
agcaacagttagggagctgt

SEQ ID NO: 105 - DNA, 23 bp, synthetic construct
  misc_feature 1..23: note = Kompetitive Allele Specific PCR (KASP) Marker
  source 1..23: mol_type = other DNA
attcatttgatctggcgatatcg

SEQ ID NO: 106 - DNA, 22 bp, synthetic construct
  misc_feature 1..22: note = Kompetitive Allele Specific PCR (KASP) Marker
  source 1..22: mol_type = other DNA
ccttttcttgaacagccaattg

SEQ ID NO: 107 - DNA, 22 bp, synthetic construct
  misc_feature 1..22: note = Kompetitive Allele Specific PCR (KASP) Marker
  source 1..22: mol_type = other DNA
ccttttcttgaacagccaatta

SEQ ID NO: 108 - DNA, 20 bp, synthetic construct
  misc_feature 1..20: note = Kompetitive Allele Specific PCR (KASP) Marker
  source 1..20: mol_type = other DNA
cttcagaaggagcttgttcg